SEQUENCE LISTING (34 sequences)

SEQ ID NO: 1 - DNA, 29 bp, Artificial Sequence
  Primer sequence
acctgcaccaactaattagggttcaaacc

SEQ ID NO: 2 - DNA, 29 bp, Artificial Sequence
  Primer sequence
ggtttgaaccctaattagttggtgcaggt

SEQ ID NO: 3 - DNA, 29 bp, Artificial Sequence
  Primer sequence
acctgcaccaactagttagggttcaaacc

SEQ ID NO: 4 - DNA, 29 bp, Artificial Sequence
  Primer sequence
ggtttgaaccctaactagttggtgcaggt

SEQ ID NO: 5 - DNA, 25 bp, Artificial Sequence
  Primer sequence
tcgctgggtggctcatggacctgca

SEQ ID NO: 6 - DNA, 25 bp, Artificial Sequence
  Primer sequence
tgcaggtccatgagccacccagcga

SEQ ID NO: 7 - DNA, 25 bp, Artificial Sequence
  Primer sequence
tcgctgggtggcacatggacctgca

SEQ ID NO: 8 - DNA, 25 bp, Artificial Sequence
  Primer sequence
tgcaggtccatgtgccacccagcga

SEQ ID NO: 9 - DNA, 29 bp, Artificial Sequence
  Primer sequence
acctgcaccaactaattagggttcaaacc

SEQ ID NO: 10 - DNA, 29 bp, Artificial Sequence
  Primer sequence
ggtttgaaccctaattagttggtgcaggt

SEQ ID NO: 11 - DNA, 29 bp, Artificial Sequence
  Primer sequence
acctgcaccaactagttagggttcaaacc

SEQ ID NO: 12 - DNA, 29 bp, Artificial Sequence
  Primer sequence
ggtttgaaccctaactagttggtgcaggt

SEQ ID NO: 13 - DNA, 25 bp, Artificial Sequence
  Primer sequence
tcgctgggtggctcatggacctgca

SEQ ID NO: 14 - DNA, 25 bp, Artificial Sequence
  Primer sequence
tgcaggtccatgagccacccagcga

SEQ ID NO: 15 - DNA, 25 bp, Artificial Sequence
  Primer sequence
tcgctgggtggcacatggacctgca

SEQ ID NO: 16 - DNA, 25 bp, Artificial Sequence
  Primer sequence
tgcaggtccatgtgccacccagcga

SEQ ID NO: 17 - DNA, 24 bp, Artificial Sequence
  Primer sequence
tttgctttctaacaatgaaaccac

SEQ ID NO: 18 - DNA, 20 bp, Artificial Sequence
  Primer sequence
ttggcacaatttaccggttt

SEQ ID NO: 19 - DNA, 20 bp, Artificial Sequence
  Primer sequence
gagttgggtgtaggggtcaa

SEQ ID NO: 20 - DNA, 20 bp, Artificial Sequence
  Primer sequence
ggttttgaacctccgcaata

SEQ ID NO: 21 - DNA, 35 bp, Artificial Sequence
  Primer sequence
tggacctgcaccaactagttagggttcaaaccaac

SEQ ID NO: 22 - DNA, 35 bp, Artificial Sequence
  Primer sequence
gttggtttgaaccctaactagttggtgcaggtcca

SEQ ID NO: 23 - DNA, 27 bp, Artificial Sequence
  Primer sequence
ctcgctgggtggcacatggacctgcac

SEQ ID NO: 24 - DNA, 27 bp, Artificial Sequence
  Primer sequence
gtgcaggtccatgtgccacccagcgag

SEQ ID NO: 25 - DNA, 20 bp, Artificial Sequence
  Primer sequence
ctagcaaaataggctgtccc

SEQ ID NO: 26 - DNA, 20 bp, Artificial Sequence
  Primer sequence
gacgatagtcatgccccgcg

SEQ ID NO: 27 - DNA, 20 bp, Artificial Sequence
  Primer sequence
cgatcagtggcttccttctc

SEQ ID NO: 28 - DNA, 20 bp, Artificial Sequence
  Primer sequence
cggacaatcctggcttttta

SEQ ID NO: 29 - DNA, 20 bp, Artificial Sequence
  Primer sequence
cgatgaccccataacattcc

SEQ ID NO: 30 - DNA, 20 bp, Artificial Sequence
  Primer sequence
aaggtctggctccatctgaa

SEQ ID NO: 31 - DNA, 20 bp, Artificial Sequence
  Primer sequence
gtcagcaacagagccctttc

SEQ ID NO: 32 - DNA, 20 bp, Artificial Sequence
  Primer sequence
ctccctctgcttggaacttg

SEQ ID NO: 33 - DNA, 20 bp, Artificial Sequence
  Primer sequence
agaacactttggggcacttg

SEQ ID NO: 34 - DNA, 20 bp, Artificial Sequence
  Primer sequence
gatttcacaccctgctgacc

(19) United States
(12) Patent Application Publication
(10) Pub. No.: US 2015/0211067 A1
(43) Pub. Date: Jul. 30, 2015

(54) NEUROPSYCHIATRIC DISORDER-ASSOCIATED MUTATIONS AND USES THEREOF

(71) Applicants: The Broad Institute, Inc., Cambridge, MA (US); President and Fellows of Harvard College, Cambridge, MA (US); Massachusetts Institute of Technology, Cambridge, MA (US)

(72) Inventors: Kerstin Lindblad-Toh, Malden, MA (US); Elinor Karlsson, Cambridge, MA (US); Hyun Ji Noh, Boston, MA (US); Guoping Feng, Newton, MA (US); Ruqi Tang, Shanghai (CN)

(21) Appl. No.: 14/605,301

(22) Filed: Jan. 26, 2015

Related U.S. Application Data
(60) Provisional application No. 61/933,176, filed on Jan. 29, 2014.

(51) Int. Cl. C12Q 1/68

(52) U.S. Cl. CPC C12Q 1/6883; C12Q 2600/156; C12Q 2600/118; C12Q 2600/16

(57) ABSTRACT

Provided herein are methods and compositions for identifying subjects as having an elevated risk of developing or having a neuropsychiatric disorder. These subjects are identified based on the presence of one or more mutations.

### CROSS-REFERENCE TO RELATED APPLICATIONS

This application claims the benefit of U.S. Provisional Application No. 61/933,176, filed Jan. 29, 2014. The entire contents of this referenced provisional application are incorporated by reference herein.

### BACKGROUND OF INVENTION

Neuropsychiatric disorders, such as obsessive-compulsive disorder, autism spectrum disorder, and Tourette syndrome, affect millions of people world-wide. Such neuropsychiatric disorders can hamper the quality of life of affected individuals. Such disorders are often inherited, but the genetic factors are not well-understood.

Obsessive-compulsive disorder (OCD), a severe neuropsychiatric disorder manifested in time-consuming repetition of behaviors, affects 1-3% of the human population. While highly heritable, complex genetics has hampered attempts to elucidate OCD etiology. Dogs also suffer from naturally occurring compulsive disorders that closely model human OCD, manifested as an excessive repetition of normal canine behaviors that only partially responds to drug therapy.

### SUMMARY OF INVENTION

The invention is premised in part on a genome-wide association study (GWAS) of 87 Doberman Pinschers with OCD and 63 controls to identify genomic loci associated with OCD or fixed in an OCD predisposed breed. These regions were then sequenced in 8 OCD-affected dogs from high-risk breeds and 8 breed-matched controls. Mutations were identified in or near several genes involved in synapse formation and function, including CDH2, CTNNA2, ATXN1, and PGCP, amongst others. Without wishing to be bound by theory, because canine neuropsychiatric disorders such as OCD are naturally-occurring models of human neuropsychiatric disorders, it is believed that the genes identified in the GWAS would also be relevant to human neuropsychiatric disorders.

Accordingly, aspects of the invention relate to methods for identifying subjects at elevated risk of developing or having a neuropsychiatric disorder (e.g., OCD).

Thus, in one aspect, this disclosure provides a method comprising: (a) analyzing genomic DNA from a subject for the presence of a mutation within or near

(i) a region having chromosomal boundaries/co-ordinates provided in Table 1 or 2, columns 5 and 6 of a gene selected from: AHNAK, ATXN1, C5orf13, CAMK4, CAPN14, CHRM1, DUSP8, EPB41L4A, FAM193A, FER, FNDC3B, GALNT14, HAUS3, KIAA0232, KIAA1530, KRTAP5-8, LRRTM1, MAN2A1, MFSD10, MOB2, MXD4, NOP14, PGCP, PHACTR1, PJA2, PLD1, SLC22A6, SLC22A8, SORCS2, STX5, TADA2B, TBC1D14, TMEM212, TMEM232, TNFSF10, TNIP2, TSPYL5, WDR36, WDR74, or ZFYVE28; or

(ii) a region having chromosomal boundaries provided in Table 2A columns 4 (human) and 6 (canine) of a gene selected from: ADD1, AHNAK, ASRGL1, ATL3, ATXN1, BLOC1S4, C4orf10, C5orf13, CAMK4, CAPN14, CCDC96, CDH2, CHRM1, CNO, CPQ, CTNNA2, DSC3, DUSP8, EPB41L4A, FAM129A, FAM193A, FER, FGFR3, FNDC3B, GALNT14, GHSR, GRPEL1, HAUS3, HCCA2, HRASLS5, INCENP, IVNS1ABP, KIAA0232, KIAA1530, KRTAP5-11, KRTAP5-2, KRTAP5-3, KRTAP5-4, KRTAP5-7, KRTAP5-8, KRTAP5-9, LETM1, LGALS12, LRRTM1, MAEA, MAN2A1, MFSD10, MOB2, MRFAP1, MXD4, NAT8L, NELFA, NOP14, NREP, PGCP, PHACTR1, PJA2, PLA2G16, PLD1, POLN, PPP2R2C, RNF2, RNF4, SCARNA22, SCGB1A1, SCGB1D1, SCGB1D2, SCGB2A1, SH3BP2, SLBP, SLC22A6, SLC22A8, SLC25A46, SLC3A2, SNHG1, SNORD22, SNORD30, SNORD31, SORCS2, STARD4, STX5, SWT1, TACC3, TADA2B, TBC1D14, TBC1D7, TMEM129, TMEM212, TMEM232, TNFSF10, TNIP2, TRMT1L, TSLP, TSPYL5, UVSSA, WDR36, WDR74, WHSC1, WHSC2, ZFYVE28; and

(b) identifying a subject having the mutation as a subject at elevated risk of developing or having a neuropsychiatric disorder.

In some embodiments, the mutation is within 100 kb, upstream or downstream, of the chromosomal boundaries/co-ordinates.

In some embodiments, the gene is selected from ATXN1, CHRM1, KIAA1530, NOP14, TMEM212, ZFYVE28, PGCP, or SLC22A8. In some embodiments, the gene is selected from ATXN1 or PGCP.

In some embodiments, the mutation is within an untranslated region (UTR), intron, or exon of the gene.

In some embodiments, the gene is ATXN1 and the mutation is within an untranslated region (UTR), intron, or exon of ATXN1. In some embodiments, the mutation is within the first intron, the 3′UTR, or intron 3 of ATXN1.

In some embodiments, the gene is PGCP and the mutation is within an untranslated region (UTR), intron, or exon of PGCP. In some embodiments, the mutation is within intron 2, exon 2, exon 5 or the 3′UTR of PGCP.

In some embodiments, the subject is a human subject. In some embodiments, the subject is a canine subject.

In some embodiments, the mutation is a SNP described in Table 3.

In some embodiments, the mutation is at least two mutations.

In some embodiments, the gene is at least two genes.

In another aspect, the disclosure provides a method comprising:

(a) analyzing genomic DNA from a subject for the presence of at least two mutations comprising a first mutation within a region having chromosomal boundaries/co-ordinates provided in Table 1 or 2 columns 5 and 6 of a first gene and a second mutation within a region having the chromosomal boundaries provided in Table 1 or 2, columns 5 and 6 of a second gene, wherein the first gene and second gene are selected from:

AHNAK, ATXN1, C5orf13, CAMK4, CAPN14, CDH2, CHRM1, CTNNA2, DUSP8, EPB41L4A, FAM193A, FER, FNDC3B, GALNT14, HAUS3, KIAA0232, KIAA1530, KRTAP5-8, LRRTM1, MAN2A1, MFSD10, MOB2, MXD4, NOP14, PGCP, PHACTR1, PJA2, PLD1, SLC22A6, SLC22A8, SORCS2, STX5, TADA2B, TBC1D14, TMEM212, TMEM232, TNFSF10, TNIP2, TSPYL5, WDR36, WDR74, or ZFYVE28; and

(b) identifying a subject having the at least two mutations as a subject at elevated risk of developing or having a neuropsychiatric disorder.

In some embodiments, the first mutation is within 100 kb (upstream or downstream) of the region of a first gene and second mutation is within 100 kb (upstream or downstream) of the region of the second gene.

In some embodiments, the first and second gene are selected from ATXN1, CDH2, CHRM1, CTNNA2, KIAA1530, NOP14, TMEM212, ZFYVE28, PGCP, or SLC22A8. In some embodiments, the first and second gene are selected from CDH2, CTNNA2, ATXN1 or PGCP.

In some embodiments, the first mutation is within an untranslated region (UTR), intron, or exon of the first gene and the second mutation is within an untranslated region (UTR), intron, or exon of the second gene.

In some embodiments, the first gene is ATXN1 and the first mutation is within an untranslated region (UTR), intron, or exon of ATXN1. In some embodiments, the first mutation is within the first intron, the 3′UTR, or intron 3 of ATXN1.

In some embodiments, the second gene is PGCP and the second mutation is within an untranslated region (UTR), intron, or exon of PGCP. In some embodiments, the second mutation is within intron 2, exon 2, exon 5 or the 3′UTR of PGCP. In some embodiments, the first gene is PGCP and the first mutation is within an untranslated region (UTR), intron, or exon of PGCP. In some embodiments, the first mutation is within intron 2, exon 2, exon 5 or the 3′UTR of PGCP.

In some embodiments, the mutation is a SNP described in Table 3.

In another aspect, the disclosure provides a method comprising

(a) analyzing genomic DNA from a subject for the presence of a mutation within the region between the genes CDH2 and DSC3; and

(b) identifying a subject having the mutation as a subject at elevated risk of developing or having a neuropsychiatric disorder.

In another aspect, the disclosure provides a method comprising

(a) analyzing genomic DNA from a subject for the presence of a mutation within intron 2 of CDH2; and

(b) identifying a subject having the mutation as a subject at elevated risk of developing or having a neuropsychiatric disorder.

In another aspect, the disclosure provides a method comprising

(a) analyzing genomic DNA from a subject for the presence of a mutation within exon 8, exon 12, exon 13, intron 7, intron 8, intron 9 or intron 12 of CTNNA2; and

(b) identifying a subject having the mutation as a subject at elevated risk of developing or having a neuropsychiatric disorder.

In another aspect, the disclosure provides a method comprising:

(a) analyzing genomic DNA from a canine subject for the presence of a SNP in Table 3 or a mutation in a region is Table 4, 5, or 6; and

(b) identifying the canine subject having the SNP or mutation as a canine subject at elevated risk of developing or having a neuropsychiatric disorder.

In some embodiments of the foregoing aspects, the subject is a human subject. In some embodiments, the subject is a canine subject.

In some embodiments of the foregoing aspects, the genomic DNA is obtained from a bodily fluid or tissue sample of the subject.

In some embodiments of the foregoing aspects, the genomic DNA is analyzed using a single nucleotide polymorphism (SNP) array. In some embodiments of the foregoing aspects, the genomic DNA is analyzed using a bead array. In some embodiments of the foregoing aspects, the genomic DNA is analyzed using a nucleic acid sequencing assay.

In some embodiments of the foregoing aspects, the method further comprises: (c) administering a therapeutic agent to the canine subject identified as at elevated risk of developing or having a neuropsychiatric disorder.

In some embodiments of the foregoing aspects, the method further comprises: (c) performing behavioral therapy on the canine subject identified as at elevated risk of developing or having a neuropsychiatric disorder.

In some embodiments of the foregoing aspects, the neuropsychiatric disorder is obsessive-compulsive disorder.

In some embodiments of the foregoing aspects, the mutation or SNP is two mutations or SNPs.

### BRIEF DESCRIPTION OF DRAWINGS

FIGS. 1A-1F show how the associated and fixed regions in Doberman Pinschers are enriched for brain-related pathways. FIG. 1A shows the original GWAS dataset showed a single peak of association at CDH2. FIG. 1B showed recalling with MAGIC yielded a 2.4× denser SNP dataset and allowed us to define 17 distinct regions of association with p<0.0001 using LD-clumping (FIG. 5), a subset of which were targeted for sequencing (dark dots above dotted line, genes labeled above peak). FIG. 1C shows that in four breeds with high rates of OCD, regions of fixation (black boxes) a subset of which was targeted for sequencing (arrowheads indicate regions that were sequenced) were identified. Sequenced regions were selected in FIG. 1D because they were large and overlapping between breeds. LD-clumping identified three distinct regions of association on chromosome 7 in FIG. 1E (boxes, with targeted regions indicated by solid lined-boxes). FIG. 1E shows the top GO gene sets enriched in the GWAS regions. GO gene sets enriched in Doberman Pinscher regions of reduced variability (RRVs) but not in 24 other breeds are shown in FIG. 1F (grey circles, most at 0).

FIGS. 2A-2C demonstrate how targeted sequencing identifies case-only variants that alter constrained elements and are more common in high OCD risk breeds. In FIG. 2A a targeted sequencing of a small number of cases and controls from four breeds (row 1) and subsequently genotyped the top candidate variants in a larger panel of dogs from those four breeds as well as two more high “OCD-risk” breeds and two low risk “control breeds” was performed (row 2). FIG. 2B demonstrates that across all variants identified in the sequencing data, the number of case-only (left box of each pair of boxes) and control-only (right box of each pair of boxes) variants is similar, but constrained elements are enriched for case-only variants. Boxes mark the 25th to 75th percentile across dogs, with the median shown as a thick line, and whiskers extending to values within 1.5 times the difference between the 25th to 75th percentiles. Outliers are marked with circles. FIG. 2C shows that case only variants have higher frequency in OCD-risk breeds and lower frequency across all genotyped breeds. The x-axis represents allele frequencies across all genotyped dogs. The y-axis represents normalized allele frequency (AF) differences between OCD-risk and control breeds ([AFOCD-risk−AFcontrol]/[AFOCD-risk+AFcontrol]). The straight downward line represents the linear model for the data points. The dark grey shade shows the 95% confidence interval for this model. Area under the curve (shaded in light grey) is notably larger in AFOCD-risk>AFcontrol than in AFOCD-risk<AFcontrol, showing that case-only variants are more common in OCD-risk breeds than in control breeds.

FIGS. 3A-3D demonstrate that a gene-based analysis identifies four genes enriched for case-only variants in constrained regions. FIG. 3A shows how genes are plotted according to the number of case-only (y-axis) and control-only (x-axis) variants within a gene and its 5 kb flanking regions from the sequence data. Squares denote all the phenotype-specific variants and the corresponding axes are shown on the top and right of the graph; diamonds denote phenotype specific variants in constrained elements only and the corresponding axes are shown on the left and bottom of the graph. Genes that are plotted above the identity line harbor more case-only than control-only variants. FIG. 3B demonstrates a similar analysis excluding Doberman Pinschers, the breed used to identify genes for sequencing, which shows that the enrichment pattern persists for several genes. The case-only variants in constrained elements, when plotted with gene structure and evolutionary conservation, show clustering in ATXN1 (5′ end) as shown in FIG. 3C. Dimmed bars represent canine variants that failed to lift over onto hg19. The conservation track shows a measure of evolutionary conservation in dog, human, mouse and rat [29]. In each gene as shown in FIG. 3D, SNPs with the greatest risk allele frequency difference between OCD-risk and control breeds (y-axis) tend to have lower frequency across all genotyped breeds (y-axis). SNPs are shown as solid circles with vertical lines.

FIGS. 4A-4E demonstrate how two intergenic case-only variants disrupt a repressor element and change gene expression in-vitro. The two case-only variants are just 20 bases apart in a 2.5 Mb gene desert between DSC3 and CDH2 on canine chromosome 7 as shown in FIG. 4A. The syntenic region of human chromosome 18, in FIG. 4B, shows markers of DNase hypersensitivity, transcription factor binding, repressor binding, histone methylation [30], and mammalian constraint [31]. In FIG. 4C, both variants, SNP55 and SNP35, alter bases that are highly constrained across mammals, SNP55 to a T and SNP35 to an A. Compositions of the four bases for each position in the 29 mammals comparison [27] are shown with different grey scale and height of bars (the higher the more conserved in multiple species). The wild-type regulatory sequence in FIG. 4D represses luciferase reporter expression in SK-N-BE neuroblastoma (2) cells. Both variants significantly change the extent of repression relative to wild-type, with SNP35-G more repressive and SNP55-A less repressive. The firefly luciferase expression in the test plasmids were normalized against the co-transfected Renilla luciferase expression in pGL4.73. P-value of the significance of the change relative to wild-type is shown above each bar, with vertical lines showing standard error of the mean. In FIG. 4E, an EMSA assay testing the wild-type alleles (lanes 1-4) and the OCD-risk alleles (lanes 5-8) of SNP35 (top gel) and SNP55 (bottom gel) show that nuclear protein binding (arrow) to SNP35 locus is disrupted by the risk allele. Nuclear extract derived from SK-N-BE (2) cells. 200-fold molar excess of competitor was used where appropriate.

FIG. 5 demonstrates regions from GWAS used for INRICH analysis (unfilled boxes) and targeted for sequencing (grey-filled boxes).

FIG. 6 shows pileup data in the region of 1.2 kb deletion at Chr29. Pileup data for the affected Jack Russell Terrier is shown on the left, while the healthy breed-matched control is shown on the right. The top track shows the coverage of the genomic region, while the lower track shows the individual reads that are aligned to this region. Abnormal reads that may be indicative of deletion [i.e. reads with an insert size larger than expected] are shown as dark grey boxes in the data.

FIG. 7 demonstrates a 1.2 kb-deletion in PGCP gene near the CCD association signal. Panel a shows −log P values for each SNP in the GWA-study in Doberman pinscher dogs. The two lines represent the signals from MAGIC (new) and BRLMM algorithm (old). Panel b shows a 1.2 kb deletion found in one Jack Russell terrier occurred in the exon2 of PGCP gene.

FIG. 8 shows DNA binding motifs at chr7:61.7 Mb detected by TRANSFAC.

FIG. 9 shows no DNA-protein binding difference between 855 (A) and 855 (T).

FIG. 10 demonstrates functional protein association network among 115 human OCD GWAS genes and CDH2, CTNNA2, ATXN1, and PGCP that were identified from the gene based analysis. The functional network was constructed using STRING [70]. 122 genes were extracted from Table 1 of the OCD GWAS by Stewart et al. [71], of which 115 genes were found in the STRING database. Note the functional connections of CDH2, CTNNA2, and ATXN1 with human OCD GWAS genes despite the generally underconnected network. The gene names, from left to right and top to bottom, are TRIM31, EFCAB3, ENSG000000214194, UBE4B, FAM129B, Z8TB34, HELT, RAD548, POP1, CCDC125, MAMSTR, C12orf62, C7orf60, QTRTD1, C8orf12, NOMO1, ELOVL7, CPEB4, RAL14, EBF3, TAAR5, ISM1, HACE1, LASSS, MAGEC2, MAGEC1, LDLRAL3, KIT, RPL12, SPTLC3, C8orf79, PMML2, TSPYL1, DCLK1, COL1A1, RPS11, LONRF1, SLC5A1, PDGFRA, RPL13A, FCGRT, GNAL, PDE4D, DLGAP1, TSKS, ACVRIC, FGF21, GRWD1, PGCP, PRMT1, HAS1, ADCY8, ZBTB43, FUT1, HMP19, TXNL1, MS4A12, GPRC6A, CXCL9, OPRD1, ZBTB20, ARHGAP18, LUC7L3, NARS, 8CDIN3D, FAJM2, BCAP31, LIFR, KRT32, AQP2, RFX6, FYB, BAMB1, C6orf91, DNAJB9, ATXN1, DLD, CAMTA1, LRSAM1, RASIP1, PKP2, TYMS, ARX, ARHGAP15, NUCB1, CYTIP, TTYH1, LRRKN3, CHX58, SPAG9, CDH2, NOSIP, TUBAIA, STXBP1, LYZL1, DPY19L4, PLK2, CTNNA2, CDH2, TUBAIA, STXBP1, NXPH3, EFNA5, IZUMO1, NOS1, K1AA0802, Corf24, GEM, FUT2, BTBD3, TNFRSF25, CRIK2, SYN1, DACH1, HIST1H2AI, and GAP43.

### DETAILED DESCRIPTION OF INVENTION

Aspects of the invention relate to mutations (such as single nucleotide polymorphisms (SNPs) and mutations in or near genes) and various methods of use and/or detection thereof. The invention is premised, in part, on the results of a GWAS that identified mutations that correlate with OCD in canines. SNPs and other types of mutations (e.g., deletions) were detected in genomic DNA samples collected from canines diagnosed with OCD. These mutations were absent or under-represented in genomic DNA samples from control canines. The identified mutations were often found within regions enriched for genes involved in synapse formation and function, such as CDH2, CTNNA2, ATXN1, and PGCP.

Accordingly, aspects of the invention provide methods that involve detecting a mutation (e.g., one or more mutations) within a region surrounding a gene (e.g., within 100 kilobases (kb) on either side of a gene) and using such detection to identify subjects having an elevated risk of developing or having a neuropsychiatric disorder.

Identifying subjects having an elevated risk of developing or having a neuropsychiatric disorder is useful in a number of applications. For example, the methods can be used for prognostic purposes and for diagnostic purposes. Accordingly, the invention provides diagnostic and prognostic methods for use in subjects, such as human subjects or canine subjects. In some embodiments, such diagnostic or prognostic methods can be paired with a treatment (e.g., a therapeutic agent or behavioral therapy).

Methods disclosed herein for identifying canine subjects have additional useful applications. For example, canine subjects identified as at elevated risk may be excluded from a breeding program and/or conversely canine subjects that do not carry the mutations may be included in a breeding program. As another example, canine subjects identified as at elevated risk may be monitored, including monitored more regularly, for the appearance of disorder-like symptoms and/or may be treated prophylactically (e.g., prior to the development of the symptoms) or therapeutically. Canine subjects carrying one or more of the mutations may also be used to further study the neuropsychiatric disorders and optionally to study the efficacy of various treatments.

### Elevated Risk of Developing a Neuropsychiatric Disorder or Having a Neuropsychiatric Disorder

The mutations of the invention can be used to identify subjects at elevated risk of developing a neuropsychiatric disorder or having a neuropsychiatric disorder. An elevated risk means a lifetime risk of developing or having such a disorder that is higher than the risk of developing or having the same disorder in (a) a population that is unselected for the presence or absence of the mutation (i.e., the general population) or (b) a population that does not carry the mutation.

### Neuropsychiatric Disorder and Diagnostic/Prognostic Methods

Aspects of the invention include various methods, such as prognostic and diagnostic methods, related to neuropsychiatric disorders. Non-limiting examples of neuropsychiatric disorders include obsessive-compulsive disorder, autism spectrum disorder, Tourette syndrome, and obsessive-compulsive spectrum such as dermatillomania, trichotillomania, and onychophagia.

Obsessive-compulsive disorder (OCD) is disorder characterized by intrusive, persistent thoughts (obsessions) and/or repetitive, intentional behaviours (compulsions) that result in significant distress or dysfunction. It affects 1 to 3% of the general population. In humans, symptoms of the disorder include excessive washing or cleaning; repeated checking; extreme hoarding; preoccupation with sexual, violent or religious thoughts; relationship-related obsessions; aversion to particular numbers; and nervous rituals, such as opening and closing a door a certain number of times before entering or leaving a room. In canines, symptoms of the disorder include excessive grooming (acral lick dermatitis), predatory behavior (tail chasing, fly snapping), eating/suckling (pica and flank sucking (FS)/blanket sucking (BS)) or locomotion (pacing/circling).

Diagnosis of OCD generally involves identifying obsessions, compulsions, or both that are “fixed” (e.g., present for a certain length of time) in a subject. Diagnosis of human subjects may be made according to the Diagnostic and Statistical Manual of Mental Disorders (DSM) or the International Classification of Diseases, 10th Edition (ICD). Obsessions include distressing ideas, images, or impulses that enter a subject's mind repeatedly. The obsessions are often violent, obscene, or perceived to be senseless and the subject finds these ideas difficult to resist. Compulsions include stereotyped behaviours that are not enjoyable that are repeated over and over and are perceived to prevent an unlikely event that is in reality unlikely to occur. The subject often recognizes that the behavior is ineffectual and makes attempts to resist it, but is unable to. Compulsions may also include repetitive behaviours or mental acts that are carried out to reduce or prevent anxiety or distress and are perceived to prevent a dreaded event or situation.

The diagnostic criteria for OCD, according to the DSM, are as follows:

1. Obsessional symptoms or compulsive acts or both must be present on most days for at least 2 successive weeks and be a source of distress or interference with activities.

2. Obsessional symptoms should have the following characteristics:
a. they must be recognized as the individual's own thoughts or impulses.
b. there must be at least one thought or act that is still resisted unsuccessfully, even though others may be present which the sufferer no longer resists.
c. the thought of carrying out the act must not in itself be pleasurable (simple relief of tension or anxiety is not regarded as pleasure in this sense).
d. the thoughts, images, or impulses must be unpleasantly repetitive.

Autism Spectrum Disorder (ASD) is a developmental disorder characterized by abnormalities in social interactions and communication, as well as restricted interests and repetitive behaviours. ASD may be diagnosed using the DSM, which provides diagnostic criteria for identifying ASD. The criteria include persistent deficits in social communication and social interaction combined with restricted, repetitive patterns of behavior, interests, or activities.

Tourette syndrome is a disorder generally having onset in childhood, characterized by multiple physical (motor) tics and at least one vocal (phonic) tic. Tourette's may be diagnosed using the DSM. The diagnostic criteria include that a person exhibits both multiple motor and one or more vocal tics (although these do not need to be concurrent) over the period of a year, with no more than three consecutive tic-free months.

Dermatillomania is characterized by the repeated urge to pick at one's own skin, often to the extent that damage is caused. Dermatillomania may be classified as an impulse control disorder by DSM-IV. Trichotillomania is characterized by compulsive urge to pull out one's own hair leading to noticeable hair loss, distress, and social or functional impairment. Trichotillomania may be classified as an impulse control disorder by DSM-IV. Onychophagia is an oral compulsive habit characterized by nail biting. Nail biting is considered an impulse control disorder in the DSM-IV-R, and is classified under obsessive-compulsive and related disorders in the DSM-5.

In some embodiments, diagnostic methods include measuring a mutation as described herein in combination with a known diagnostic method (e.g., a behavioral test or use of a questionnaire or assessment provided in DSM IV, DSM IV-R, or DSM 5).

### Mutations

Aspects of the invention relate to a (i.e., at least one) mutation and uses and detection thereof in various methods. As used herein, a mutation is one or more changes in the nucleotide sequence of the genome of the subject. As used herein, mutations include, but are not limited to, point mutations (e.g., SNPs), insertions, deletions, rearrangements, inversions and duplications. Mutations also include, but are not limited to, silent mutations, missense mutations, and nonsense mutations. In some embodiments, the mutation is a SNP. SNPs are further described herein.

The mutation can be a germ-line mutation or a somatic mutation. In some embodiments, the mutation is a germ-line mutation. A germ-line mutation is generally found in the majority, if not all, of the cells in a subject. Germ-line mutations are generally inherited from one or both parents of the subject (i.e., were present in the germ cells of one or both parents). Germ-line mutations as used herein also include de novo germ-line mutations, which are spontaneous mutations that occur at single-cell stage level during development. A somatic mutation occurs after the single-cell stage during development. Somatic mutations are considered to be spontaneous mutations. Somatic mutations generally originate in a single cell or subset of cells in the subject.

A mutation as described herein may be found within a gene described herein or within a region encompassing such a gene (e.g., a region that encompasses the gene as well as 100 kb or more upstream and 100 kb or more downstream of the gene).

### Genes

In some embodiments, a mutation provided herein is a mutation within or near a gene. In some embodiments, the gene is a gene provided in Tables 1, 2 and/or 2A. The boundaries of each gene are defined using the “start” and “end” coordinates provided in columns 3 and 4 respectively of Tables 1 and 2 for canine and human subjects, respectively, and in columns 4 and 6 of Table 2A for canine and human subjects, respectively. It is to be understood that these coordinates are inclusive (i.e., including the boundaries).

The start and end coordinates (i.e., the chromosome coordinates) and the Ensembl Gene IDs in Table 1 are based on the CanFam 2.0 genome assembly (CF2, see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). However, certain Ensembl Gene IDs in Table 1 are from CanFam3 as indicated by a “*” in Table 1. The genes with Ensembl Gene IDs from CanFam3 still are indicated using coordinates from CanFam2. One of skill in the art can use the information from CanFam2 to determine the corresponding coordinates in CanFam3.

The start and end coordinates (i.e., the chromosome coordinates) in Table 2 are based on the 19^{th} human genome assembly (Hg19, see, e.g., UCSC Genome Browser). For both CF2 and Hg19, the first base pair in each chromosome is labeled 0 and the position of the start and end is then the number of base pairs from the first base pair. Similar designations apply to Table 2A.

A gene may include regulatory sequences (e.g., promoters, enhancers, or suppressors, either adjacent to or far from the coding sequence) and coding sequences. As used herein, a coding sequence includes the first DNA nucleotide to the last DNA nucleotide that is transcribed into an mRNA that includes the untranslated regions (UTRs), exons, and introns. The coding sequence for each gene can be obtained using the Ensembl database by entering the Ensembl gene IDs provided in Tables 1, 2 and 2A, or by other methods known in the art. In some embodiments, the mutation is within or near (e.g., within 100 kb of) the coding sequence of a gene. Thus, it is to be understood that this disclosure provides for detecting mutations within or near “genes” or within or near coding sequence, and that although many embodiments are described relative to “gene” co-ordinates this is only for the sake of brevity only and that the disclosure contemplates and provides parallel embodiments relative to coding sequence (and its co-ordinates) as well.

In some embodiments, a mutation, such as a SNP, is contained within or near the gene. In some embodiments, the mutation is within 5000 kb, 2500 kb, 1000 kb, 900 kb, 800 kb, 700 kb, 600 kb, 500 kb, 400 kb, 300 kb, 200 kb, 150 kb, 100 kb, 50 kb, 25 kb, 10 kb, or 5 kb of a gene or of the coding sequence of the gene, as described herein. In some embodiments, a mutation is contained within the boundaries provided in the “start−100 kb (or more)” column and the “end+100 kb (or more)” column of Table 1 or Table 2.

TABLE 1
Canine Genes

| | | | | CF2 gene | CF2 gene | |
|---|---|---|---|---|---|---|
| Gene/ | | | | start | end | Canine |
| alternative | CF2 | CF2 gene | CF2 gene | −100 kb | +100 kb | Ensembl ID CF2 |
| name | Chr | start (bp) | end (bp) | (or more) | (or more) | (*CF3) |
| AHNAK | 18 | 57111508 | 57204953 | 57011508 | 57304953 | ENSCAFG00000032525* |
| ATXN1 | 35 | 18051982 | 19120649 | 17951982 | 19220649 | ENSCAFG00000009934 |
| C5orf13/ | 3 | 3716109 | 3905519 | 3616109 | 4005519 | ENSCAFG00000029015* |
| NREP | | | | | | |
| CAMK4 | 3 | 4066930 | 4255863 | 3966930 | 4355863 | ENSCAFG00000007379* |
| CAPN14 | 17 | 27876735 | 27910671 | 27776735 | 28010671 | ENSCAFG00000005335* |
| CDH2/ | 7 | 63770673 | 63954411 | 61575628 | 64054411 | ENSCAFG00000018115 |
| NCAD/ | | | | | | |
| CDHN/ | | | | | | |
| CD325 | | | | | | |
| CHRM1 | 18 | 56811105 | 56824613 | 56711105 | 56924613 | ENSCAFG00000015467* |
| CTNNA2 | 17 | 46579258 | 47624001 | 45869829 | 48358242 | ENSCAFG00000029212* |
| DSC3 | 7 | 61307878 | 61437524 | 61207878 | 61575628 | ENSCAFG00000018089* |
| DUSP8 | 18 | 48819295 | 48837382 | 48719295 | 48937382 | ENSCAFG00000010009* |
| EPB41L4A | 3 | 3393380 | 3573255 | 3293380 | 3673255 | ENSCAFG00000007361* |
| FAM193A | 3 | 64363640 | 64437727 | 64263640 | 64537727 | ENSCAFG00000014845* |
| FER | 3 | 5753085 | 6181378 | 5653085 | 6281378 | ENSCAFG00000007431* |
| FNDC3B | 34 | 39380380 | 39647266 | 39280380 | 39747266 | ENSCAFG00000015349* |
| GALNT14 | 17 | 27638086 | 27843913 | 27538086 | 27943913 | ENSCAFG00000005328* |
| HAUS3 | 3 | 64765514 | 64771175 | 64665514 | 64871175 | ENSCAFG00000014883* |
| KIAA0232 | 3 | 61730720 | 61794063 | 61630720 | 61894063 | ENSCAFG00000014342* |
| KIAA1530/ | 3 | 65473890 | 65497580 | 65373890 | 65597580 | ENSCAFG00000015039* |
| UVSSA | | | | | | |
| KRTAP5-8 | 18 | 48938143 | 48939464 | 48838143 | 49039464 | ENSCAFG00000010018 |
| LRRTM1 | 17 | 46900213 | 46902709 | 46800213 | 47002709 | ENSCAFG00000008098* |
| MAN2A1 | 3 | 5227722 | 5386233 | 5127722 | 5486233 | ENSCAFG00000007417* |
| MFSD10 | 3 | 64171974 | 64175917 | 64071974 | 64275917 | ENSCAFG00000014747* |
| MOB2/ | 18 | 48725507 | 48743299 | 48625507 | 48843299 | ENSCAFG00000010003* |
| HCCA2 | | | | | | |
| MXD4 | 3 | 64746990 | 64760994 | 64646990 | 64860994 | ENSCAFG00000014873* |
| NOP14 | 3 | 64150945 | 64170455 | 64050945 | 64270455 | ENSCAFG00000014730* |
| PGCP/CPQ | 29 | 44129705 | 44448160 | 43964205 | 44548160 | ENSCAFG00000009482* |
| PHACTR1 | 35 | 15198859 | 15752279 | 15098859 | 15852279 | ENSCAFG00000009796 |
| PJA2 | 3 | 5636486 | 5682511 | 5536486 | 5782511 | ENSCAFG00000007425* |
| PLD1 | 34 | 38924126 | 39127169 | 38824126 | 39227169 | ENSCAFG00000015246* |
| SLC22A6 | 18 | 56763493 | 56771285 | 56663493 | 56871285 | ENSCAFG00000015418* |
| SLC22A8 | 18 | 56736483 | 56755457 | 56636483 | 56855457 | ENSCAFG00000024439* |
| SORCS2 | 3 | 62042751 | 62497792 | 61942751 | 62597792 | ENSCAFG00000014403* |
| STX5 | 18 | 56889396 | 56909986 | 56789396 | 57009986 | ENSCAFG00000015540* |
| TADA2B | 3 | 61928464 | 61942793 | 61828464 | 62042793 | ENSCAFG00000014357* |
| TBC1D14 | 3 | 61812574 | 61919473 | 61712574 | 62019473 | ENSCAFG00000014349* |
| TMEM212 | 34 | 39155866 | 39166233 | 39055866 | 39266233 | ENSCAFG00000015285* |
| TMEM232 | 3 | 4617023 | 4827260 | 4517023 | 4927260 | ENSCAFG00000007400* |
| TNFSF10 | 34 | 39723435 | 39741399 | 39623435 | 39841399 | ENSCAFG00000015383* |
| TNIP2 | 3 | 64331070 | 64355389 | 64231070 | 64455389 | ENSCAFG00000014836* |
| TSPYL5 | 29 | 44487036 | 44490633 | 44387036 | 44590633 | ENSCAFG00000032518* |
| WDR36 | 3 | 4334033 | 4378201 | 4234033 | 4478201 | ENSCAFG00000007387* |
| WDR74 | 18 | 56882021 | 56888561 | 56782021 | 56988561 | ENSCAFG00000015523* |
| ZFYVE28 | 3 | 64624524 | 64738876 | 64524524 | 64838876 | ENSCAFG00000014866* |

Chr = chromosome

TABLE 2
Human Genes

| Gene/ | | | | HG19 start | HG19 end | |
|---|---|---|---|---|---|---|
| alternative | HG19 | HG19 start | HG19 end | -100 kb | +30100 kb | Human Ensembl |
| name | Chr | (bp) | (bp) | (or more) | (or more) | ID |
| AHNAK | 11 | 62201016 | 62323707 | 62101016 | 62423707 | ENSG00000124942 |
| ATXN1 | 6 | 16299343 | 16761722 | 15861792 | 17076499 | ENSG00000124788 |
| C5orf13/ | 5 | 110998318 | 111333161 | 110898318 | 111433161 | ENSG00000134986 |
| NREP | | | | | | |
| CAMK4 | 5 | 110559351 | 110830584 | 110459351 | 110930584 | ENSG00000152495 |
| CAPN14 | 2 | 31395924 | 31456724 | 31295924 | 31556724 | ENSG00000214711 |
| CDH2 | 18 | 25530930 | 25757410 | 25430930 | 28410850 | ENSG00000170558 |
| CHRM1 | 11 | 62676151 | 62689279 | 62576151 | 62789279 | ENSG00000168539 |
| CTNNA2 | 2 | 79412357 | 80875905 | 78758590 | 81721993 | ENSG00000066032 |
| DSC3 | 18 | 28,570,052 | 28,622,781 | 28410850 | 28,722,781 | ENSG00000134762 |
| DUSP8 | 11 | 1575274 | 1593150 | 1475274 | 1693150 | ENSG00000184545 |
| EPB41L4A | 5 | 111478138 | 111755013 | 111378138 | 111855013 | ENSG00000129595 |
| FAM193A | 4 | 2626988 | 2734292 | 2526988 | 2834292 | ENSG00000125386 |
| FER | 5 | 108083523 | 108532542 | 107983523 | 108632542 | ENSG00000151422 |
| FNDC3B | 3 | 171757418 | 172119455 | 171657418 | 172219455 | ENSG00000075420 |
| GALNT14 | 2 | 31133333 | 31378068 | 31033333 | 31478068 | ENSG00000158089 |
| HAUS3 | 4 | 2229191 | 2243891 | 2129191 | 2343891 | ENSG00000214367 |
| KIAA0232 | 4 | 6783102 | 6885897 | 6683102 | 6985897 | ENSG00000170871 |
| KIAA1530/ | 4 | 1341054 | 1381837 | 1241054 | 1481837 | ENSG00000163945 |
| UVSSA | | | | | | |
| KRTAP5-8 | 11 | 71249071 | 71250253 | 71149071 | 71350253 | ENSG00000241233 |
| LRRTM1 | 2 | 80515483 | 80531874 | 80415483 | 80631874 | ENSG00000162951 |
| MAN2A1 | 5 | 109025067 | 109205326 | 108925067 | 109305326 | ENSG00000112893 |
| MFSD10 | 4 | 2932288 | 2936586 | 2832288 | 3036586 | ENSG00000109736 |
| MOB2/ | 11 | 1490687 | 1522477 | 1390687 | 1622477 | ENSG00000182208 |
| HCCA2 | | | | | | |
| MXD4 | 4 | 2249159 | 2264021 | 2149159 | 2364021 | ENSG00000123933 |
| NOP14 | 4 | 2939660 | 2965112 | 2839660 | 3065112 | ENSG00000087269 |
| PGCP/CPQ | 8 | 97657455 | 98161882 | 97557455 | 98661717 | ENSG00000104324 |
| PHACTR1 | 6 | 12717893 | 13288645 | 12617893 | 13388645 | ENSG00000112137 |
| PJA2 | 5 | 108670410 | 108745695 | 108570410 | 108845695 | ENSG00000198961 |
| PLD1 | 3 | 171318195 | 171528740 | 171218195 | 171628740 | ENSG00000075651 |
| SLC22A6 | 11 | 62703857 | 62752455 | 62603857 | 62852455 | ENSG00000197901 |
| SLC22A8 | 11 | 62756626 | 62783311 | 62656626 | 62883311 | ENSG00000149452 |
| SORCS2 | 4 | 7194265 | 7744554 | 7094265 | 7844554 | ENSG00000184985 |
| STX5 | 11 | 62574369 | 62599560 | 62474369 | 62699560 | ENSG00000162236 |
| TADA2B | 4 | 7043626 | 7059679 | 6943626 | 7159679 | ENSG00000173011 |
| TBC1D14 | 4 | 6910969 | 7034845 | 6810969 | 7134845 | ENSG00000132405 |
| TMEM212 | 3 | 171561139 | 171656505 | 171461139 | 171756505 | ENSG00000186329 |
| TMEM232 | 5 | 109624934 | 110074657 | 109524934 | 110174657 | ENSG00000186952 |
| TNFSF10 | 3 | 172223298 | 172241297 | 172123298 | 172341297 | ENSG00000121858 |
| TNIP2 | 4 | 2743375 | 2758103 | 2643375 | 2858103 | ENSG00000168884 |
| TSPYL5 | 8 | 98285717 | 98290176 | 98185717 | 98390176 | ENSG00000180543 |
| WDR36 | 5 | 110427414 | 110466200 | 110327414 | 110566200 | ENSG00000134987 |
| WDR74 | 11 | 62599814 | 62609281 | 62499814 | 62709281 | ENSG00000133316 |
| ZFYVE28 | 4 | 2271309 | 2420390 | 2171309 | 2542442 | ENSG00000159733 |

TABLE 2A
Additional Human and Canine Genes, Ensembl Gene IDs and Regions

| | Alternate | | Dog genome | | |
|---|---|---|---|---|---|
| | Gene | | build canFam2 | Human Ensembl | Human genome build |
| Gene | Name | Dog Ensembl Gene ID | location | Gene Id | hg19 location |
| EPB41L4A | | ENSCAFG00000007361 | chr3: 3393380- | ENSG00000129595 | chr5: 111478138- |
| | | | 3573255 | | 111755013 |
| NREP | C5orf13 | ENSCAFG00000029015 | chr3: 3701249- | ENSG00000134986 | chr5: 110998318- |
| | | | 3950185 | | 111333161 |
| STARD4 | | ENSCAFG00000030762 | chr3: 4040880- | ENSG00000164211 | chr5: 110831731- |
| | | | 4058155 | | 110848288 |
| CAMK4 | | ENSCAFG00000007379 | chr3: 4066930- | ENSG00000152495 | chr5: 110559351- |
| | | | 4255863 | | 110830584 |
| WDR36 | | ENSCAFG00000007387 | chr3: 4334033- | ENSG00000134987 | chr5: 110427414- |
| | | | 4378201 | | 110466200 |
| TSLP | | ENSCAFG00000031005 | chr3: 4387317- | ENSG00000145777 | chr5: 110405760- |
| | | | 4395157 | | 110413722 |
| SLC25A46 | | ENSCAFG00000007396 | chr3: 4588808- | ENSG00000164209 | chr5: 110073837- |
| | | | 4605819 | | 110100857 |
| TMEM232 | | ENSCAFG00000007400 | chr3: 4617023- | ENSG00000186952 | chr5: 109624934- |
| | | | 4827260 | | 110074657 |
| MAN2A1 | | ENSCAFG00000007417 | chr3: 5227722- | ENSG00000112893 | chr5: 109025067- |
| | | | 5386233 | | 109205326 |
| PJA2 | | ENSCAFG00000007425 | chr3: 5636486- | ENSG00000198961 | chr5: 108670410- |
| | | | 5682511 | | 108745695 |
| FER | | ENSCAFG00000007431 | chr3: 5753085- | ENSG00000151422 | chr5: 108083523- |
| | | | 6181378 | | 108532542 |
| PPP2R2C | | ENSCAFG00000014257 | chr3: 61342316- | ENSG00000074211 | chr4: 6322305-6565327 |
| | | | 61522973 | | |
| MRFAP1 | | ENSCAFG00000032015 | chr3: 61579691- | ENSG00000179010 | chr4: 6641818-6644472 |
| | | | 61582026 | | |
| CNO | BLOC1S4 | ENSCAFG00000014337 | chr3: 61642361- | ENSG00000186222 | chr4: 6717842-6719387 |
| | | | 61643017 | | |
| KIAA0232 | | ENSCAFG00000014342 | chr3: 61730720- | ENSG00000170871 | chr4: 6783102-6885897 |
| | | | 61794063 | | |
| TBC1D14 | | ENSCAFG00000014349 | chr3: 61812574- | ENSG00000132405 | chr4: 6910969-7034845 |
| | | | 61919473 | | |
| CCDC96 | | ENSCAFG00000014356 | chr3: 61924886- | ENSG00000173013 | chr4: 7042579-7044728 |
| | | | 61926365 | | |
| TADA2B | | ENSCAFG00000014357 | chr3: 61928464- | ENSG00000173011 | chr4: 7043626-7059679 |
| | | | 61942793 | | |
| GRPEL1 | | ENSCAFG00000014364 | chr3: 61943146- | ENSG00000109519 | chr4: 7060633-7069924 |
| | | | 61951444 | | |
| SORCS2 | | ENSCAFG00000014403 | chr3: 62042751- | ENSG00000184985 | chr4: 7194265-7744554 |
| | | | 62497792 | | |
| NOP14 | | ENSCAFG00000014730 | chr3: 64150945- | ENSG00000087269 | chr4: 2939660-2965112 |
| | | | 64170455 | | |
| C4orf10 | NOP14- | none | chr3: 64152650- | ENSG00000249673 | chr4: 2936626-2963465 |
| | AS1 | | 64171936 | | |
| MFSD10 | | ENSCAFG00000014747 | chr3: 64171974- | ENSG00000109736 | chr4: 2932288-2936586 |
| | | | 64175917 | | |
| ADD1 | | ENSCAFG00000014808 | chr3: 64176551- | ENSG00000087274 | chr4: 2845584-2931803 |
| | | | 64263307 | | |
| SH3BP2 | | ENSCAFG00000014831 | chr3: 64271300- | ENSG00000087266 | chr4: 2794750-2842825 |
| | | | 64308858 | | |
| TNIP2 | | ENSCAFG00000014836 | chr3: 64331070- | ENSG00000168884 | chr4: 2743375-2758103 |
| | | | 64355389 | | |
| FAM193A | | ENSCAFG00000014845 | chr3: 64363640- | ENSG00000125386 | chr4: 2626988-2734292 |
| | | | 64437727 | | |
| RNF4 | | ENSCAFG00000014856 | chr3: 64437863- | ENSG00000063978 | chr4: 2463947-2627047 |
| | | | 64585406 | | |
| ZFYVE28 | | ENSCAFG00000014866 | chr3: 64624524- | ENSG00000159733 | chr4: 2271309-2420390 |
| | | | 64738876 | | |
| MXD4 | | ENSCAFG00000014873 | chr3: 64746990- | ENSG00000123933 | chr4: 2249159-2264021 |
| | | | 64760994 | | |
| HAUS3 | | ENSCAFG00000014883 | chr3: 64765514- | ENSG00000214367 | chr4: 2229191 -2243891 |
| | | | 64771175 | | |
| POLN | | ENSCAFG00000014903 | chr3: 64765526- | ENSG00000130997 | chr4: 2073645-2243848 |
| | | | 64921721 | | |
| NAT8L | | ENSCAFG00000014913 | chr3: 64923446- | ENSG00000185818 | chr4: 2061239-2070816 |
| | | | 64932942 | | |
| WHSC2 | NELFA | ENSCAFG00000014927 | chr3: 64941264- | ENSG00000185049 | chr4: 1984441-2043630 |
| | | | 64983654 | | |
| WHSC1 | | ENSCAFG00000014951 | chr3: 64984077- | ENSG00000109685 | chr4: 1873151-1983934 |
| | | | 65071512 | | |
| SCARNA22 | | ENSCAFG00000028375 | chr3: 64990166- | ENSG00000249784 | chr4: 1976363-1976487 |
| | | | 64990290 | | |
| LETM1 | | ENSCAFG00000014979 | chr3: 65086989- | ENSG00000168924 | chr4: 1813206-1857974 |
| | | | 65123651 | | |
| FGFR3 | | ENSCAFG00000014993 | chr3: 65128333- | ENSG00000068078 | chr4: 1795034-1810599 |
| | | | 65143108 | | |
| TACC3 | | ENSCAFG00000015000 | chr3: 65187852- | ENSG00000013810 | chr4: 1723227-1746898 |
| | | | 65200023 | | |
| TMEM129 | | ENSCAFG00000015011 | chr3: 65200106- | ENSG00000168936 | chr4: 1717679-1723085 |
| | | | 65205587 | | |
| SLBP | | ENSCAFG00000015016 | chr3: 65205959- | ENSG00000163950 | chr4: 1694527-1714282 |
| | | | 65220745 | | |
| UVSSA | KIAA1530 | ENSCAFG00000015039 | chr3: 65471712- | ENSG00000163945 | chr4: 1341054-1381837 |
| | | | 65497614 | | |
| MAEA | | ENSCAFG00000015056 | chr3: 65504893- | ENSG00000090316 | chr4: 1283639-1333935 |
| | | | 65541084 | | |
| FAM129A | | ENSCAFG00000013490 | chr7: 20983574- | ENSG00000135842 | chr1: 184759858- |
| | | | 21138582 | | 184943682 |
| RNF2 | | ENSCAFG00000013499 | chr7: 21197694- | ENSG00000121481 | chr1: 185014496- |
| | | | 21246203 | | 185071740 |
| TRMT1L | | ENSCAFG00000013518 | chr7: 21256205- | ENSG00000121486 | chr1: 185087220- |
| | | | 21292700 | | 185126204 |
| SWT1 | | ENSCAFG00000013543 | chr7: 21292708- | ENSG00000116668 | chr1: 185126212- |
| | | | 21387710 | | 185260897 |
| IVNS1ABP | | ENSCAFG00000013589 | chr7: 21392920- | ENSG00000116679 | chr1: 185265520- |
| | | | 21414401 | | 185286461 |
| DSC3 | | ENSCAFG00000018089 | chr7: 61307878- | ENSG00000134762 | chr18: 28569974- |
| | | | 61437524 | | 28622781 |
| CDH2 | NCAD, | ENSCAFG00000018115 | chr7: 63770673- | ENSG00000170558 | chr18: 25530930- |
| | CDHN, | | 63954411 | | 25757410 |
| | CD325 | | | | |
| GALNT14 | | ENSCAFG00000005328 | chr17: 27638086- | ENSG00000158089 | chr2: 31133333- |
| | | | 27843913 | | 31378068 |
| CAPN14 | | ENSCAFG00000005335 | chr17: 27876735- | ENSG00000214711 | chr2: 31395924- |
| | | | 27910671 | | 31456724 |
| CTNNA2 | | ENSCAFG00000029212 | chr17: 46579258- | ENSG00000066032 | chr2: 79412357- |
| | | | 47624001 | | 80875905 |
| LRRTM1 | | ENSCAFG00000008098 | chr17: 46900213- | ENSG00000162951 | chr2: 80515483- |
| | | | 46902709 | | 80531874 |
| HCCA2 | MOB2 | ENSCAFG00000010003 | chr18: 48725507- | ENSG00000182208 | chr11: 1490687- |
| | | | 48743299 | | 1522477 |
| DUSP8 | | ENSCAFG00000010009 | chr18: 48819295- | ENSG00000184545 | chr11: 1575274- |
| | | | 48837382 | | 1593150 |
| KRTAP5-3 | | none | chr18: 48850311- | ENSG00000196224 | chr11: 1628795- |
| | | | 48924741 | | 1629693 |
| KRTAP5-4 | | none | chr18: 48856910- | ENSG00000241598 | chr11: 1642188- |
| | | | 48924741 | | 1643368 |
| KRTAP5-7 | | none | chr18: 48871593- | ENSG00000244411 | chr11: 71238313- |
| | | | 48924741 | | 71239210 |
| KRTAP5-9 | | none | chr18: 48871593- | ENSG00000254997 | chr11: 71259466- |
| | | | 48909090 | | 71260653 |
| KRTAP5-2 | | none | ch18: 48871605- | ENSG00000205867 | chr11: 1618409- |
| | | | 48994265 | | 1619524 |
| KRTAP5- | | none | chr18: 48908447- | ENSG00000204571 | chr11: 71292901- |
| 11 | | | 48994265 | | 71314399 |
| KRTAP5-8 | | ENSCAFG00000010018 | chr18: 48938143- | ENSG00000241233 | chr11: 71249071- |
| | | | 48939464 | | 71250253 |
| ATL3 | | ENSCAFG00000015073 | chr18: 56334456- | ENSG00000184743 | chr11: 63391559- |
| | | | 56391919 | | 63439393 |
| PLA2G16 | | ENSCAFG00000015082 | chr18: 56395570- | ENSG00000176485 | chr11: 63340667- |
| | | | 56427212 | | 63384355 |
| LGALS12 | | ENSCAFG00000015091 | chr18: 56465483- | ENSG00000133317 | chr11: 63273556- |
| | | | 56474619 | | 63284246 |
| HRASLS5 | | ENSCAFG00000015096 | chr18: 56478882- | ENSG00000168004 | chr11: 63228876- |
| | | | 56491956 | | 63258666 |
| SLC22A8 | | ENSCAFG00000024439 | chr18: 56736483- | ENSG00000149452 | chr11: 62756626- |
| | | | 56755457 | | 62783311 |
| SLC22A6 | | ENSCAFG00000015418 | chr18: 56763493- | ENSG00000197901 | chr11: 62703857- |
| | | | 56771285 | | 62752455 |
| M1 | | ENSCAFG00000015467 | chr18: 56811105- | ENSG00000168539 | chr11: 62676151- |
| | | | 56824613 | | 62689279 |
| SLC3A2 | | ENSCAFG00000015495 | chr18: 56837111- | ENSG00000168003 | chr11: 62623518- |
| | | | 56868211 | | 62656352 |
| SNORD22 | | ENSCAFG00000026245 | chr18: 56870415- | ENSG00000252365 | chr13: 94021100- |
| | | | 56870541 | | 94021213 |
| SNORD30 | | ENSCAFG00000025801 | chr18: 56871003- | ENSG00000212611 | chr11: 62621136- |
| | | | 56871067 | | 62621199 |
| SNORD31 | | ENSCAFG00000022180 | chr18: 56871335- | ENSG00000201847 | chr13: 107973243- |
| | | | 56871403 | | 107973311 |
| WDR74 | | ENSCAFG00000015523 | chr18: 56882021- | ENSG00000133316 | chr11: 62599814- |
| | | | 56888561 | | 62609281 |
| STX5 | | ENSCAFG00000015540 | chr18: 56889396- | ENSG00000162236 | chr11: 62574369- |
| | | | 56909986 | | 62599560 |
| AHNAK | | ENSCAFG00000032525 | chr18: 57111508- | ENSG00000124942 | chr11: 62201016- |
| | | | 57204953 | | 62323707 |
| SCGB1A1 | | ENSCAFG00000015860 | chr18: 57213844- | ENSG00000149021 | chr11: 62172575- |
| | | | 57228076 | | 62190667 |
| ASRGL1 | | ENSCAFG00000015868 | chr18: 57242327- | ENSG00000162174 | chr11: 62104920- |
| | | | 57264479 | | 62160882 |
| SCGB1D2 | | ENSCAFG00000029389 | chr18: 57286656- | ENSG00000124935 | chr11: 62009682- |
| | | | 57289929 | | 62012280 |
| SCGB2A1 | | ENSCAFG00000032725 | chr18: 57339758- | ENSG00000124939 | chr11: 61976140- |
| | | | 57342653 | | 61981408 |
| SCGB1D1 | | ENSCAFG00000029389 | chr18: 57361513- | ENSG00000168515 | chr11: 61957688- |
| | | | 57364270 | | 61961011 |
| INCENP | | ENSCAFG00000015888 | chr18: 57385990- | ENSG00000149503 | chr11: 61891445- |
| | | | 57410795 | | 61920635 |
| PGCP | CPQ | ENSCAFG00000009482 | chr29: 44129705- | ENSG00000104324 | chr8: 97657455- |
| | | | 44448160 | | 98161882 |
| TSPYL5 | | ENSCAFG00000032518 | chr29: 44487036- | ENSG00000180543 | chr8: 98285717- |
| | | | 44490633 | | 98290176 |
| PLD1 | | ENSCAFG00000015246 | chr34: 38924126- | ENSG00000075651 | chr3: 171318195- |
| | | | 39127169 | | 171528740 |
| TMEM212 | | ENSCAFG00000015285 | chr34: 39155866- | ENSG00000186329 | chr3: 171561139- |
| | | | 39166233 | | 171656505 |
| FNDC3B | | ENSCAFG00000015349 | chr34: 39380380- | ENSG00000075420 | chr3: 171757418- |
| | | | 39647266 | | 172119455 |
| GHSR | | ENSCAFG00000015376 | chr34: 39697513- | ENSG00000121853 | chr3: 172162923- |
| | | | 39700712 | | 172166246 |
| TNFSF10 | | ENSCAFG00000015383 | chr34: 39723435- | ENSG00000121858 | chr3: 172223298- |
| | | | 39741399 | | 172241297 |
| PHACTR1 | | ENSCAFG00000009796 | chr35: 15198859- | ENSG00000112137 | chr6: 12717893- |
| | | | 15752279 | | 13288645 |
| TBC1D7 | | ENSCAFG00000009827 | chr35: 15737568- | ENSG00000145979 | chr6: 13266774- |
| | | | 15799270 | | 13328815 |
| ATXN1 | | ENSCAFG00000009934 | chr35: 18051982- | ENSG00000124788 | chr6: 16299343- |
| | | | 19120649 | | 16761722 |

Table 2A provides start and end co-ordinates for a variety of human and canine genes. It is to be understood that the disclosure further contemplates detection of mutations some distance upstream or downstream of these start and end co-ordinates respectively, as for example is elaborated in Tables 1 and 2.

In some embodiments, a mutation in a gene or within a region encompassing a gene (e.g., a region that includes the gene plus 100 kb or 150 kb upstream and 100 kb or 150 kb downstream of the gene) is used in the methods described herein. In some embodiments, the method comprises:

(a) analyzing genomic DNA from a subject for the presence of a mutation (i) within a gene (e.g., within and including the start and end coordinates provided in columns 3 and 4 of Table 1 or 2 or columns 4 and 6 of Table 2A) and/or (ii) near a gene (e.g., within 150 kb, 100 kb, 50 kb, 25 kb, 10 kb, or 5 kb of the start and end coordinates provided in columns 3 and 4 of Table 1 or 2 or in columns 4 and 6 of Table 2A) and/or (iii) within and including the coordinates provided in columns 5 and 6 of Table 1 or 2); and

(b) identifying a subject having the mutation as a subject at elevated risk of developing or having a neuropsychiatric disorder. It is to be understood that the start and end coordinates in Table 1 and 2 are coordinates on the chromosome number provided in column 2.

It is to be understood that any number of mutations (e.g., 1, 2, 3, 4, 5, 6, 7, 8, 9, 10, 11, 12, 13, 14, 15, 16, 17, 18, 19, or 20 or more mutations) in or near any number of genes (e.g., 1, 2, 3, 4, 5, 6, 7, 8, 9, 10, 11, 12, 13, 14, 15, 16, 17, 18, 19, or 20 or more genes) are contemplated. Any mutation of any size located within or near a gene is contemplated herein, e.g., a SNP, a deletion, an inversion, a translocation, or a duplication. In some embodiments, the mutation is a SNP.

In some embodiments, the mutation is within or near a gene, wherein the gene is selected from ATXN1, CDH2, CHRM1, CTNNA2, KIAA1530, NOP14, TMEM212, ZFYVE28, PGCP, or SLC22A8. In some embodiments, 1, 2, 3, 4, 5, 6, 7, 8, 9 or 10 (or more) mutations are within or near 1, 2, 3, 4, 5, 6, 7, 8, 9 or 10 genes, wherein the genes are 1, 2, 3, 4, 5, 6, 7, 8, 9, or all 10 of ATXN1, CDH2, CHRM1, CTNNA2, KIAA1530, NOP14, TMEM212, ZFYVE28, PGCP, and SLC22A8. In some embodiments, CTNNA2 and CDH2 are excluded.

In some embodiments, the mutation is within or near a gene, wherein the gene is selected from ATXN1, CDH2, CTNNA2, or PGCP. In some embodiments, 1, 2, 3, or 4 (or more) mutations are within or near 1, 2, 3, or 4 genes, wherein the genes are 1, 2, 3, or all 4 of ATXN1, CDH2, CTNNA2, and PGCP. In some embodiments, CTNNA2 and CDH2 are excluded.

In some embodiments, a mutation is within or near CDH2. In some embodiments, the mutation is within or near CDH2, with the proviso that the mutation is not within an exon of CDH2. In some embodiments, the mutation is within an intron or UTR of CDH2. In some embodiments, a mutation is within the chromosomal region between the genes CDH2 and DSC3.

### SNPs and Chromosomal Regions

In some embodiments, a mutation provided herein is a single nucleotide polymorphism (SNP). A SNP is a mutation that occurs at a single nucleotide location on a chromosome. The nucleotide located at that position may differ between individuals in a population and/or paired chromosomes in an individual.

In some embodiments, the subject is a canine subject and the mutation is a (at least one) SNP selected from Table 3. The risk nucleotide is the nucleotide identity that is associated with elevated risk of developing or having a neuropsychiatric disorder. The positions (i.e., the chromosome coordinates) in Table 3 are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The first base pair in each chromosome is labeled 0 and the position of the SNP is then the number of base pairs from the first base pair.

TABLE 3
Canine SNPs

| CHR | Location | Non-risk | Risk |
|---|---|---|---|
| chr3 | 3494592 | A | G |
| chr3 | 3894924 | G | A |
| chr3 | 3896965 | G | A |
| chr3 | 3967754 | G | C |
| chr3 | 4256410 | C | T |
| chr3 | 4380380 | T | C |
| chr3 | 4380394 | A | G |
| chr3 | 4604531 | C | A |
| chr3 | 5470809 | T | C |
| chr3 | 5471514 | A | G |
| chr3 | 5559403 | A | G |
| chr3 | 5559765 | A | G |
| chr3 | 5681788 | G | T |
| chr3 | 5754697 | G | A |
| chr3 | 5754700 | G | A |
| chr3 | 6063747 | A | G |
| chr3 | 61823869 | T | C |
| chr3 | 64690526 | G | A |
| chr3 | 64769048 | C | A |
| chr3 | 65188233 | A | G |
| chr3 | 65472187 | G | A |
| chr3 | 65472276 | G | A |
| chr7 | 61669045 | T | A |
| chr7 | 61693835 | T | C |
| chr7 | 61693855 | A | T |
| chr7 | 61693952 | G | C |
| chr7 | 61722312 | G | A |
| chr7 | 61728453 | T | C |
| chr7 | 61865715 | G | A |
| chr7 | 63779775 | C | G |
| chr7 | 63794041 | A | G |
| chr7 | 63796857 | C | A |
| chr7 | 63796858 | G | T |
| chr7 | 63802530 | C | T |
| chr7 | 63806661 | A | G |
| chr7 | 63814172 | C | T |
| chr7 | 63814306 | G | A |
| chr7 | 63814541 | A | C |
| chr7 | 63832008 | A | G |
| chr7 | 63845160 | A | T |
| chr7 | 63845290 | C | T |
| chr7 | 63852056 | C | T |
| chr7 | 63852467 | T | C |
| chr7 | 63857947 | C | T |
| chr7 | 63860234 | G | A |
| chr7 | 63866105 | C | A |
| chr7 | 63866151 | G | A |
| chr7 | 63866863 | G | T |
| chr7 | 63867146 | T | A |
| chr7 | 63867472 | C | T |
| chr7 | 63867472 | C | T |
| chr7 | 63867618 | G | C |
| chr7 | 63867879 | T | A |
| chr7 | 63868034 | C | T |
| chr7 | 63868258 | T | C |
| chr7 | 63868442 | T | C |
| chr7 | 63870150 | G | A |
| chr7 | 63870467 | A | G |
| chr7 | 63870482 | C | A |
| chr7 | 63870496 | A | G |
| chr7 | 63870599 | A | G |
| chr7 | 63870805 | T | A |
| chr7 | 63872172 | G | C |
| chr7 | 63891778 | C | T |
| chr7 | 63912017 | G | A |
| chr7 | 63921141 | T | C |
| chr7 | 63943045 | G | A |
| chr7 | 63943118 | T | A |
| chr7 | 63950125 | G | A |
| chr7 | 63966490 | C | A |
| chr17 | 27881676 | C | T |
| chr17 | 46478790 | T | C |
| chr17 | 46607594 | T | C |
| chr17 | 46617340 | G | A |
| chr17 | 46715746 | T | A |
| chr17 | 46722666 | C | G |
| chr17 | 46781268 | T | C |
| chr17 | 46781433 | G | A |
| chr17 | 46781512 | G | A |
| chr17 | 46791139 | G | C |
| chr17 | 46791238 | T | C |
| chr17 | 46791415 | C | T |
| chr17 | 46897529 | A | G |
| chr18 | 48821291 | C | T |
| chr18 | 48822346 | A | T |
| chr18 | 48823350 | A | G |
| chr18 | 48901967 | G | A |
| chr18 | 48938780 | C | T |
| chr18 | 56737581 | C | T |
| chr18 | 56754830 | G | A |
| chr18 | 56768794 | C | T |
| chr18 | 56889625 | C | T |
| chr18 | 56898066 | A | G |
| chr18 | 57136368 | T | A |
| chr18 | 57174849 | G | A |
| chr18 | 57225591 | C | G |
| chr19 | 13696914 | T | C |
| chr29 | 44152594 | A | C |
| chr29 | 44177940 | G | A |
| chr29 | 44180170 | C | A |
| chr29 | 44205912 | T | C |
| chr29 | 44205914 | A | G |
| chr29 | 44205937 | A | G |
| chr29 | 44205943 | G | A |
| chr29 | 44244625 | A | G |
| chr29 | 44249614 | T | C |
| chr29 | 44300177 | G | A |
| chr29 | 44306347 | G | A |
| chr29 | 44306628 | A | G |
| chr29 | 44334298 | A | G |
| chr29 | 44336600 | G | T |
| chr29 | 44338785 | G | A |
| chr29 | 44348091 | C | T |
| chr29 | 44353724 | C | T |
| chr29 | 44392126 | A | G |
| chr29 | 44392979 | C | T |
| chr29 | 44393030 | A | G |
| chr29 | 44394199 | C | T |
| chr29 | 44397446 | T | G |
| chr29 | 44408722 | C | T |
| chr29 | 44422867 | T | G |
| chr29 | 44437957 | T | C |
| chr29 | 44447800 | C | T |
| chr29 | 44489802 | A | C |
| chr29 | 44513249 | A | G |
| chr34 | 38925589 | C | T |
| chr34 | 38956076 | A | G |
| chr34 | 38986422 | G | A |
| chr34 | 39405162 | G | A |
| chr34 | 39420664 | G | T |
| chr35 | 18464093 | T | A |
| chr35 | 18484947 | A | G |
| chr35 | 18565131 | T | C |
| chr35 | 18679978 | G | C |
| chr35 | 18850625 | T | G |
| chr35 | 18851783 | G | C |
| chr35 | 18857719 | T | A |
| chr35 | 18860763 | C | T |
| chr35 | 18861596 | G | A |
| chr35 | 18862817 | G | A |

Chr = chromosome

In some embodiments, the SNP is chr7:61865715, chr7:61693835 and/or chr7:61693855.

In some embodiments, a SNP can be used in the methods described herein. In some embodiments, the method comprises:

(a) analyzing genomic DNA from a subject for the presence of a SNP (e.g., a SNP in Table 3); and

(b) identifying a subject having the SNP as a subject at elevated risk of developing or having a neuropsychiatric disorder.

Any number of SNPs are contemplated herein, e.g., 1, 2, 3, 4, 5, 6, 7, 8, 9, 10, 11, 12, 13, 14, 15, 16, 17, 18, 19, 20 or more SNPs.

In some embodiments, the subject is a canine subject and the mutation is located within a chromosomal region provided in Table 4, 5, and/or 6. The positions (i.e., the chromosome coordinates) in Tables 4, 5, and 6 are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The first base pair in each chromosome is labeled 0 and the position of the boundary is then the number of base pairs from the first base pair.

TABLE 4
Chromosomal Regions

| region | size (kb) | genes associated with region |
|---|---|---|
| chr7 63741207- | 213 | CDH2 |
| 63954411 | | |
| chr7 61392736- | 507 | DSC3 |
| 61900158 | | |
| chr34 38431782- | 1340 | TNIK, PLD1, TMEM212, FNDC3B, GHSR, TNFSF10 |
| 39772222 | | |
| chr26 38766783- | 1235 | DKK1, PRKG1 |
| 40001941 | | |
| chr17 27638084- | 292 | GALNT14, CAPN14 |
| 27929679 | | |
| chr18 56127466- | 644 | MARK2, C11orf84, C18H11orf84, C11orf95, RTN3, ATL3, PLA2G16, |
| 56771285 | | HRASLS2, RARRES3, LGALS12, HRASLS5, SLC22A9, SLC22A24, |
| | | SLC22A25, SLC22A10, SLC22A8, SLC22A6 |
| chr29 43925364- | 523 | SDC2, PGCP, CPQ |
| 44448160 | | |
| chr18 48725507- | 321 | MOB2, DUSP8, KRTAP5-11, KRTAP5-2, KRTAP5-9, KRTAP5-8, |
| 49046739 | | KRTAP5-3, KRTAP5-4, KRTAP5-7, KRTAP5-6, IFITM10, CTSD |
| chr35 18409969- | 463 | GMPR, ATXN1 |
| 18872470 | | |
| chr18 57242322- | 168 | ASRGL1, SCGB1D2, SCGB1D1, SCGB2A1, INCENP |
| 57410796 | | |
| chr7 20881499- | 1338 | EDEM3, FAM129A, RNF2, TRMT1L, SWT1, IVNS1ABP, HMCN1 |
| 22219043 | | |
| chr17 57571016- | 474 | TRIM45, VTCN1, MAN1A2, FAM46C |
| 58045329 | | |
| chr38 20737417- | 1461 | LRRC52, RXRG, LMX1A, PBX1 |
| 22198037 | | |
| chr7 60919881- | 305 | TTR, DSG2, DSG3, DSG4, DSG1 |
| 61225006 | | |
| chr35 14998490- | 801 | PHACTR1, TBC1D7 |
| 15799243 | | |
| chr38 19827135- | 259 | TAF1A, MIA3, AIDA, BROX, FAM177B |
| 20086439 | | |
| chr24 29224804- | 1335 | |
| 30560165 | | |

TABLE 5
Further Chromosomal Regions

| | | | | # | |
|---|---|---|---|---|---|
| chr | start | end | size | genes | genes |
| 3 | 62948826 | 70302993 | 7.35 | 57 | ACOX3, METTL19, TRMT44, GPR78, CPZ, HMX1, ADRA2C, |
| | | | | | LRPAP1, DOK7, HGFAC, RGS12, HTT, GRK4, NOP14, |
| | | | | | MFSD10, ADD1, SH3BP2, TNIP2, FAM193A, RNF4, ZFYVE28, |
| | | | | | MXD4, HAUS3, POLN, NAT8L, C4orf48, WHSC2, NELFA, |
| | | | | | WHSC1, LETM1, FGFR3, TACC3, TMEM129, SLBP, FAM53A, |
| | | | | | UVSSA, MAEA, FAM184B, MED28, LAP3, CLRN2, QDPR, |
| | | | | | LDB2, TAPT1, PROM1, FGFBP1, CD38, BST1, FAM200B, |
| | | | | | FBXL5, CC2D2A, C1QTNF7, CPEB2, BOD1L, BOD1L1, NKX3- |
| | | | | | 2, RAB28 |
| 3 | 59513417 | 62897146 | 3.38 | 33 | MESDC2, KIAA1199, FAM108C1, ARNT2, FAH, ZFAND6, |
| | | | | | BCL2A1, MTHFS, KIAA1024, TMED3, RASGRF1, CTSH, |
| | | | | | MORF4L1, ADAMTS7, TBC1D2B, IDH3A, ACSBG1, DNAJA4, |
| | | | | | WDR61, CRABP1, PPP2R2C, MRFAP1, S100P, BLOC1S4, |
| | | | | | KIAA0232, TBC1D14, TADA2B, GRPEL1, SORCS2, PSAPL1, |
| | | | | | AFAP1, ABLIM2, SH3TC1 |
| 3 | 3548237 | 6087635 | 2.54 | 11 | EPB41L4A, NREP, STARD4, CAMK4, WDR36, TSLP, |
| | | | | | SLC25A46, TMEM232, MAN2A1, PJA2, FER |
| 24 | 3013164 | 4715848 | 1.70 | 10 | CST8, CST11, CSTL1, NAPB, GZF1, NXT1, CD93, THBD, |
| | | | | | SSTR4, FOXA2 |
| 31 | 7605474 | 9218454 | 1.61 | 1 | GBE1 |
| 17 | 45925444 | 47203813 | 1.28 | 2 | CTNNA2, LRRTM1 |
| 31 | 3075862 | 4279751 | 1.20 | 6 | CGGBP1, ZNF654, HTR1F, POU1F1, CHMP2B, VGLL3 |
| 12 | 36982027 | 38147867 | 1.17 | 2 | RIMS1, KCNQ5 |
| 14 | 3737103 | 4832311 | 1.10 | 17 | IBA57, GJC2, GUK1, MRPL55, ARF1, WNT3A, WNT9A, |
| | | | | | PRSS38, SNAP47, OR6F1, OR13G1, OR2AK2, OR2L13, |
| | | | | | OR2L3, OR2W3, TRIM58, OR11L1 |
| 2 | 62546927 | 63611219 | 1.06 | 13 | BBS2, OGFOD1, NUDT21, AMFR, GNAO1, CES5A, CES1, |
| | | | | | CES1P1, SLC6A2, LPCAT2, CAPNS2, MMP2, IRX6 |
| 13 | 57506475 | 58481113 | 0.97 | 1 | TECRL |
| 13 | 6295550 | 7242655 | 0.95 | 6 | GRHL2, NCALD, RRM2B, UBR5, ODF1, KLF10 |
| 11 | 49214235 | 50070536 | 0.86 | 1 | LINGO2 |

TABLE 6
Further Chromosomal Regions

| CHR | START | END | GENES |
|---|---|---|---|
| 2 | 19440000 | 19590000 | none |
| 2 | 21780000 | 21990000 | MIR511-1, MIR511-2, SLC39A12, MRC1 |
| 2 | 80760000 | 80970000 | EIF4G3 |
| 3 | 66030000 | 66210000 | none |
| 3 | 68820000 | 68970000 | none |
| 4 | 6660000 | 6870000 | EDARADD, ERO1LB, GPR137B |
| 4 | 91290000 | 91440000 | ANKH |
| 5 | 12930000 | 13140000 | OR8B2, OR8B3, OR8B4, OR8B8 |
| 5 | 17160000 | 17370000 | PVRL1 |
| 5 | 42210000 | 42390000 | TRIM16L, ZNF286A, ZNF287, ZNF624 |
| 5 | 45690000 | 45930000 | KCNJ12, MAP2K3 |
| 5 | 81840000 | 81990000 | none |
| 6 | 12510000 | 12660000 | AZGP1, COPS6, ZKSCAN1, ZNF3, ZSCAN21 |
| 6 | 14100000 | 14250000 | BHLHA15, LMTK2, TECPR1 |
| 6 | 14610000 | 14760000 | C7orf70, CYTH3, FAM220A, RAC1 |
| 6 | 39630000 | 39900000 | SEPT12, ANKS3, FAM100A, GLYR1, MGRN1, ROGDI, ZNF500 |
| 7 | 55320000 | 55530000 | none |
| 7 | 69300000 | 69450000 | ABHD3, MIB1, SNRPD1 |
| 8 | 9540000 | 9690000 | none |
| 8 | 11910000 | 12120000 | PRKD1 |
| 8 | 19680000 | 19860000 | none |
| 8 | 21060000 | 21240000 | none |
| 8 | 25110000 | 25320000 | none |
| 8 | 33330000 | 33480000 | CDKN3, CGRRF1, CNIH, GMFB |
| 8 | 60810000 | 61020000 | none |
| 8 | 67320000 | 67500000 | C14orf49, GLRX5, SNHG10 |
| 8 | 70860000 | 71010000 | CCDC85C, CCNK, SETD3 |
| 8 | 71310000 | 71490000 | EML1, EVL, MIR342 |
| 9 | 7890000 | 8040000 | LLGL2, RECQL5, SAP30BP, TSEN54 |
| 9 | 15450000 | 15720000 | BPTF, CEP95, SMURF2 |
| 9 | 16110000 | 16320000 | PITPNC1, PSMD12 |
| 9 | 18660000 | 18840000 | ABCA10, ABCA5, ABCA6, ABCA9 |
| 9 | 51030000 | 51180000 | ATP2A3, CACNA1B |
| 10 | 11580000 | 11730000 | IRAK3, LLPH, TMBIM4 |
| 10 | 31920000 | 32070000 | HMGXB4, ISX |
| 11 | 6270000 | 6420000 | AGXT2L2, COL23A1, HNRNPAB, N4BP3, NHP2, RMND5B |
| 11 | 36690000 | 36870000 | MPDZ |
| 11 | 38310000 | 38490000 | FREM1 |
| 11 | 41850000 | 42030000 | FAM154A, HAUS6, PLIN2, RRAGA, SCARNA8 |
| 11 | 52230000 | 52380000 | none |
| 12 | 14340000 | 14490000 | CNPY3, PTCRA, RPL7L1 |
| 12 | 19500000 | 19650000 | none |
| 12 | 35490000 | 35640000 | COL19A1 |
| 12 | 36660000 | 36930000 | LINC00472, MIR30A, MIR30C2, OGFRL1 |
| 12 | 38220000 | 38370000 | KCNQ5, KHDC1, KHDC1L |
| 12 | 63780000 | 64050000 | none |
| 12 | 65940000 | 66120000 | none |
| 13 | 5880000 | 6030000 | ZNF706 |
| 13 | 45210000 | 45360000 | GABRA2, GABRG1 |
| 13 | 53490000 | 53700000 | none |
| 13 | 61080000 | 61230000 | GNRHR, UBA6 |
| 14 | 48390000 | 48540000 | BBS9 |
| 15 | 3390000 | 3600000 | C1orf50, ERMAP, LOC100129924, SLC2A1 |
| 15 | 6870000 | 7110000 | MYCBP, RRAGC |
| 15 | 16290000 | 16440000 | CMPK1, STIL, FOXE3 |
| 15 | 19950000 | 20100000 | EBNA1BP2, WDR65 |
| 15 | 24450000 | 24600000 | SYT1 |
| 15 | 28170000 | 28350000 | none |
| 15 | 33930000 | 34140000 | none |
| 15 | 40650000 | 40860000 | none |
| 15 | 53460000 | 53730000 | ARFIP1, TIGD4, TMEM154 |
| 15 | 63900000 | 64050000 | TRIM61 |
| 15 | 65520000 | 65790000 | SPOCK3 |
| 16 | 56040000 | 56190000 | none |
| 17 | 18060000 | 18210000 | LAPTM4A, SDC1 |
| 17 | 46440000 | 46650000 | CTNNA2 |
| 17 | 65040000 | 65310000 | LOR, MAGI3 |
| 18 | 7920000 | 8130000 | HPVC1 |
| 18 | 15390000 | 15630000 | LAMB1, LAMB4, NRCAM |
| 18 | 51570000 | 51780000 | MYEOV |
| 19 | 13680000 | 13830000 | none |
| 19 | 21390000 | 21540000 | ANXA5 |
| 19 | 21630000 | 21780000 | QRFPR |
| 19 | 23460000 | 23610000 | FAM123C |
| 19 | 37080000 | 37230000 | DPP10 |
| 20 | 8580000 | 8790000 | C3orf25, CAND2, H1FOO, IFT122, PLXND1, RPL32, RHO |
| 20 | 16050000 | 16260000 | ITPR1, SETMAR, SUMF1 |
| 20 | 17580000 | 17790000 | CNTN4 |
| 20 | 20160000 | 20340000 | none |
| 20 | 23970000 | 24120000 | FOXP1 |
| 20 | 32190000 | 32370000 | PTPRG |
| 20 | 33480000 | 33660000 | FHIT |
| 20 | 61020000 | 61200000 | BSG, C2CD4D, CDC34, FGF22, FSTL3, HCN2, MADCAM1, ODF3L2, POLRMT, |
| | | | PRSS57, RNF126, SHC2, THEG, TPGS1 |
| 21 | 51570000 | 51780000 | KIF18A, METTL15 |
| 22 | 20250000 | 20400000 | none |
| 22 | 29160000 | 29340000 | none |
| 22 | 32220000 | 32400000 | COMMD6, LMO7, TBC1D4, UCHL3 |
| 22 | 47310000 | 47520000 | GPC6 |
| 23 | 8850000 | 9000000 | ARPP21 |
| 23 | 18480000 | 18630000 | none |
| 23 | 24480000 | 24750000 | ZNF385D |
| 24 | 3960000 | 4140000 | LINC00261, FOXA2 |
| 24 | 10560000 | 10740000 | MACROD2 |
| 24 | 44490000 | 44670000 | AURKA, C20orf43, CASS4, CSTF1, FAM210B, GCNT7 |
| 25 | 28860000 | 29100000 | CTSB, DEFB131, DEFB134, DEFB135, DEFB136, FDFT1, LOC100129216, NEIL2 |
| 25 | 39720000 | 39900000 | ATP6V1B2, SLC18A1, LZTS1 |
| 25 | 49350000 | 49500000 | AGAP1 |
| 25 | 53250000 | 53520000 | GPC1, MIR149, MYEOV2, OTOS, PP14571 |
| 26 | 3630000 | 3780000 | GALNT9, LOC100130238 |
| 26 | 4260000 | 4410000 | GPR133 |
| 26 | 24270000 | 24570000 | MN1, PITPNB, TTC28, TTC28-AS1 |
| 26 | 33300000 | 33510000 | CCDC74A, CCDC74B, KLHL22, MED15, MZT2A, MZT2B, SCARF2, SMPD4, |
| | | | TUBA3C |
| 26 | 33990000 | 34200000 | MAPK1, PPIL2, YPEL1 |
| 26 | 38880000 | 39030000 | PRKG1 |
| 26 | 40800000 | 41010000 | ATAD1, KLLN, PAPSS2, PTEN |
| 27 | 3780000 | 3960000 | GPR84, GTSF1, ITGA5, NCKAP1L, PDE1B, ZNF385A |
| 27 | 21480000 | 21660000 | ERGIC2, OVCH1, TMTC1 |
| 27 | 37260000 | 37440000 | ETV6 |
| 28 | 35730000 | 36000000 | none |
| 28 | 36330000 | 36480000 | CHST15, CPXM2 |
| 28 | 39990000 | 40140000 | none |
| 29 | 19080000 | 19230000 | C8orf46, MYBL1, VCPIP1 |
| 29 | 20910000 | 21060000 | C8orf34 |
| 29 | 32580000 | 32730000 | none |
| 29 | 33600000 | 33840000 | RALYL |
| 30 | 14850000 | 15060000 | PLDN, SLC30A4, SQRDL |
| 30 | 22650000 | 22800000 | UNC13C |
| 30 | 37710000 | 37890000 | CT62, LRRC49, THSD4 |
| 32 | 14340000 | 14490000 | ABCG2, PKD2 |
| 33 | 12600000 | 12780000 | none |
| 33 | 33060000 | 33210000 | DLG1 |
| 34 | 10110000 | 10260000 | LOC255167, NSUN2, SRD5A1, UBE2QL1 |
| 34 | 16830000 | 17010000 | CCDC39, TTC14 |
| 34 | 32670000 | 32820000 | none |
| 36 | 7200000 | 7350000 | CCDC148, PKP4 |
| 36 | 21270000 | 21510000 | CIR1, GPR155, OLA1, SCRN3 |
| 38 | 19470000 | 19740000 | none |
| 35 | 3240000 | 3510000 | none |

In some embodiments, a mutation is located within a chromosomal region selected from chr3:62948826-70302993, chr3:3548237-6087635, chr24:3013164-4715848, chr17:45925444-47203813, chr2:21780000-21990000, chr5:17160000-17370000, chr6:14610000-14760000, chr10:11580000-11730000, chr13:45210000-45360000, chr15:16290000-16440000, chr15:24450000-24600000, chr17:46440000-46650000, chr18:15390000-15630000, chr20:8580000-8790000, chr20:16050000-16260000, chr20:61020000-61200000, chr24:3960000-4140000, chr25:39720000-39900000, chr26:33990000-34200000, chr26:40800000-41010000, chr27:3780000-3960000, or chr36:7200000-7350000.

Any chromosomal coordinates described herein are meant to be inclusive (i.e., include the boundaries of the chromosomal coordinates). In some embodiments, the chromosomal region provided in Tables 4, 5 and/or 6 may include additional chromosomal regions flanking those chromosomal regions described above, e.g., an additional 0.1, 0.5, 1, 2, 3, 4 or 5 Mb. In some embodiments, the chromosomal region may be a shortened chromosomal region than those chromosomal regions described above, e.g., 0.1, 0.5, or 1 Mb fewer than the chromosomal regions described above.

Any mutation of any size located within or spanning the chromosomal boundaries of a chromosomal region is contemplated herein, e.g., a SNP, a deletion, an inversion, a translocation, or a duplication. In some embodiments, the mutation is a SNP. In some embodiments, a SNP in a SNP described in Table 3 having chromosome coordinates within the chromosomal region. It is to be understood that other SNPs not listed in Table 4 but located within the chromosomal coordinates are also contemplated herein.

In some embodiments, a mutation in a chromosomal region can be used in the methods described herein. In some embodiments, the method comprises:

(a) analyzing genomic DNA from a subject for the presence of a mutation in a chromosomal region (e.g., a chromosomal region described in Table 4, 5, and/or 6); and

(b) identifying the subject having the mutation as a subject at elevated risk of developing or having a neuropsychiatric disorder.

It is to be understood that any number of mutations (e.g., 1, 2, 3, 4, 5, 6, 7, 8, 9, 10, 11, 12, 13, 14, 15, 16, 17, 18, 19, or 20 or more mutations) can exist within each chromosomal region. It is also to be understood that any number of chromosomal regions is contemplated herein (e.g., 1, 2, 3, 4, 5, 6, 7, 8, 9, 10, 11, 12, 13, 14, 15, 16, 17, 18, 19, or 20 or more chromosomal regions).

### Genome Analysis Methods

Methods provided herein comprise analyzing genomic DNA. In some embodiments, analyzing genomic DNA comprises carrying out a nucleic acid-based assay, such as a sequencing-based assay or a hybridization-based assay. In some embodiments, the genomic DNA is analyzed using a single nucleotide polymorphism (SNP) array. In some embodiments, the genomic DNA is analyzed using a bead array. Methods of genetic analysis are known in the art. Examples of genetic analysis methods and commercially available tools are described below.

Affymetrix:

The Affymetrix SNP 6.0 array contains over 1.8 million SNP and copy number probes on a single array. The method utilizes at a simple restriction enzyme digestion of 250 ng of genomic DNA, followed by linker-ligation of a common adaptor sequence to every fragment, a tactic that allows multiple loci to be amplified using a single primer complementary to this adaptor. Standard PCR then amplifies a predictable size range of fragments, which converts the genomic DNA into a sample of reduced complexity as well as increases the concentration of the fragments that reside within this predicted size range. The target is fragmented, labeled with biotin, hybridized to microarrays, stained with streptavidin-phycoerythrin and scanned. To support this method, Affymetrix Fluidics Stations and integrated GS-3000 Scanners can be used.

Illumina Infinium:

Examples of commercially available Infinium array options include the 660W-Quad (>660,000 probes), the 1MDuo (over 1 million probes), and the custom iSelect (up to 200,000 SNPs selected by user). Samples begin the process with a whole genome amplification step, then 200 ng is transferred to a plate to be denatured and neutralized, and finally plates are incubated overnight to amplify. After amplification the samples are enzymatically fragmented using end-point fragmentation. Precipitation and resuspension clean up the DNA before hybridization onto the chips. The fragmented, resuspended DNA samples are then dispensed onto the appropriate BeadChips and placed in the hybridization oven to incubate overnight. After hybridization the chips are washed and labeled nucleotides are added to extend the primers by one base. The chips are immediately stained and coated for protection before scanning. Scanning is done with one of the two Illumina iScan™ Readers, which use a laser to excite the fluorophore of the single-base extension product on the beads. The scanner records high-resolution images of the light emitted from the fluorophores. All plates and chips are barcoded and tracked with an internally derived laboratory information management system. The data from these images are analyzed to determine SNP genotypes using Illumina's BeadStudio. To support this process, Biomek F/X, three Tecan Freedom Evos, and two Tecan Genesis Workstation 150s can be used to automate all liquid handling steps throughout the sample and chip prep process.

Illumina BeadArray:

The Illumina Bead Lab system is a multiplexed array-based format. Illumina's BeadArray Technology is based on 3-micron silica beads that self-assemble in microwells on either of two substrates: fiber optic bundles or planar silica slides. When randomly assembled on one of these two substrates, the beads have a uniform spacing of ˜5.7 microns. Each bead is covered with hundreds of thousands of copies of a specific oligonucleotide that act as the capture sequences in one of Illumina's assays. BeadArray technology is utilized in Illumina's iScan System.

Sequenom:

During pre-PCR, either of two Packard Multiprobes is used to pool oligonucleotides, and a Tomtec Quadra 384 is used to transfer DNA. A Cartesian nanodispenser is used for small-volume transfer in pre-PCR, and another in post-PCR. Beckman Multimeks, equipped with either a 96-tip head or a 384-tip head, are used for more substantial liquid handling of mixes. Two Sequenom pin-tool are used to dispense nanoliter volumes of analytes onto target chips for detection by mass spectrometry. Sequenom Compact mass spectrometers can be used for genotype detection.

In some embodiments, methods provided herein comprise analyzing genomic DNA using a nucleic acid sequencing assay. Methods of genome sequencing are known in the art. Examples of genome sequencing methods and commercially available tools are described below.

Illumina Sequencing:

89 GAIIx Sequencers are used for sequencing of samples. Library construction is supported with 6 Agilent Bravo plate-based automation, Stratagene MX3005p qPCR machines, Matrix 2-D barcode scanners on all automation decks and 2 Multimek Automated Pipettors for library normalization.

454 Sequencing:

Roche® 454 FLX-Titanium instruments are used for sequencing of samples. Library construction capacity is supported by Agilent Bravo automation deck, Biomek FX and Janus PCR normalization.

SOLiD Sequencing:

SOLiD v3.0 instruments are used for sequencing of samples. Sequencing set-up is supported by a Stratagene MX3005p qPCR machine and a Beckman SC Quanter for bead counting.

ABI Prism® 3730 XL Sequencing:

ABI Prism® 3730 XL machines are used for sequencing samples. Automated Sequencing reaction set-up is supported by 2 Multimek Automated Pipettors and 2 Deerac Fluidics-Equator systems. PCR is performed on 60 Thermo-Hybaid 384-well systems.

Ion Torrent:

Ion PGM™ or Ion Proton™ machines are used for sequencing samples. Ion library kits (Invitrogen) can be used to prepare samples for sequencing.

Other Technologies:

Examples of other commercially available platforms include Helicos Heliscope Single-Molecule Sequencer, Polonator G.007, and Raindance RDT 1000 Rainstorm.

### Controls

Some of the methods provided herein involve determining the presence or absence a mutation in a biological sample and then comparing that presence or absence to a control in order to identify a subject having an elevated risk of developing or having a neuropsychiatric disorder. The control may be the identity of the nucleic acid(s) at the corresponding location in a control tissue, control subject, or a population of control subjects.

The control may be (or may be derived from) a normal subject (or normal subjects). A normal subject, as used herein, refers to a subject that is healthy, such a subject experiencing none of the symptoms associate with a neuropsychiatric disorder. The control population may be a population of normal subjects.

In other instances, the control may be (or may be derived from) a subject (a) having a similar neuropsychiatric disorder to that of the subject being tested and (b) who is negative for the mutation.

It is to be understood that the methods provided herein do not require that a control identity be measured every time a subject is tested. Rather, it is contemplated that control identities are obtained and recorded and that any test identity is compared to such a pre-determined identity.

In some embodiments, the mutation is a SNP described in Table 3 and the control is a nucleotide other than the risk nucleotide as described in Table 3.

### Samples

The methods provided herein detect and optionally measure (and thus analyze) particular mutations in biological samples. Biological samples, as used herein, refer to samples taken or obtained from a subject. These biological samples may be tissue samples or they may be fluid samples (e.g., bodily fluid). Examples of biological fluid samples are whole blood, plasma, serum, urine, sputum, phlegm, saliva, tears, and other bodily fluids. In some embodiments, the biological sample is a whole blood or saliva sample. In some embodiments, the biological sample is a biopsy sample, e.g., a central nervous system biopsy sample.

In some embodiments, the biological sample may comprise a polynucleotide (e.g., genomic DNA or mRNA) derived from a tissue sample or fluid sample of the subject. In some embodiments, the biological sample may be manipulated to extract a polynucleotide. In some embodiments, the biological sample may be manipulated to amplify a polynucleotide sample. Methods for extraction and amplification (e.g., PCR) are well known in the art.

### Subjects

Methods of the invention are intended for human and canine subjects. In some embodiments, canine subjects include, for example, those with a higher incidence of a neuropsychiatric disorder as determined by breed. For example, the canine subject may be a Doberman pinscher, bull terrier, Shetland sheepdog, German shepherd, or Jack Russell terrier, or a descendant of a Doberman pinscher, bull terrier, Shetland sheepdog, German shepherd, or Jack Russell terrier. As used herein, a “descendant” includes any blood relative in the line of descent, e.g., first generation, second generation, third generation, fourth generation, etc., of a canine subject. Such a descendant may be a pure-bred canine subject, or a mixed-breed canine subject. Breed can be determined, e.g., using commercially available genetic tests (see, e.g., Wisdom Panel).

In some embodiments, a subject (e.g., a subject identified in a method herein) is at elevated risk of developing or having a neuropsychiatric disorder. In some embodiments, a subject (e.g., a subject identified in a method herein) has a neuropsychiatric disorder.

It is to be understood that methods of the invention may be used in a variety of other subjects including but not limited to mammals such as humans, canines, felines, mice, rats, rabbits, and apes.

### Computational Analysis

Methods of computation analysis of genomic and expression data are known in the art. Examples of available computational programs are: Genome Analysis Toolkit (GATK, Broad Institute, Cambridge, Mass.), Expressionist Refiner module (Genedata AG, Basel, Switzerland), GeneChip-Robust Multichip Averaging (CG-RMA) algorithm, PLINK (Purcell et al, 2007), GCTA (Yang et al, 2011), the EIGENSTRAT method (Price et al 2006), EMMAX (Kang et al, 2010). In some embodiments, methods described herein include a step comprising computational analysis.

### Breeding Programs

Other aspects of the invention relate to use of the diagnostic methods, when the subject is a canine subject, in connection with a breeding program. A breeding program is a planned, intentional breeding of a group of animals to reduce detrimental or undesirable traits and/or increase beneficial or desirable traits in offspring of the animals. Thus, a subject identified using the methods described herein as not having a mutation of the invention may be included in a breeding program to reduce the risk of developing a neuropsychiatric disorder in the offspring of said subject. Alternatively, a subject identified using the methods described herein as having a mutation of the invention may be excluded from a breeding program. In some embodiments, methods of the invention comprise exclusion of a subject identified as being at elevated risk of developing or having a neuropsychiatric disorder in a breeding program or inclusion of a subject identified as not being at elevated risk of developing or having a neuropsychiatric disorder in a breeding program.

### Treatment

Other aspects of the invention relate to diagnostic or prognostic methods that comprise a treatment step (also referred to as “theranostic” methods due to the inclusion of the treatment step). Any treatment for a neuropsychiatric disorder is contemplated. In some embodiments, treatment comprises behavioral therapy and/or one or more therapeutic agents.

In some embodiments, treatment comprises administration of an effective amount of an appropriate therapeutic agent for the particular neuropsychiatric disorder, e.g., an antidepressant, a stimulant, an antidopaminergic, or a central adrenergic inhibitor. Non-limiting examples of antidepressants include ariprazole, doxepin, clomipramine, bupropion, amoxapine, nortriptyline, citalopram, duloxetine, trazodone, venlafaxine, selegiline, amitriptyline, escitalopram, isocarboxazid, phenelzine, desipramine, trazodone, nortriptyline, tranylcypromine, paroxetine, paroxetine, fluoxetine, desvenlafaxine, mirtazapine, fluoxetine, quetiapine, nefazodone, doxepin, trimipramine, imipramine, vilazodone, protriptyline, bupropion, sertraline, and olanzapine. Non-limiting examples of antidopaminergics include domperidone, haloperidol, chlorpromazine and alizapride. Non-limiting examples of stimulants include Adderall, Adderall XR, Concerta, Dexedrine, Dexedrine spansule, Daytrana, Metadate CD, Metadate ER, Methylin ER, Ritalin, Ritalin LA, Ritalin SR, Vyvanse, and Quillivant XR. Non-limiting examples of central adrenergic inhibitors include clonidine and guanfacine.

In some embodiments, the neuropsychiatric disorder is OCD. Non-limiting examples of therapeutic agents for OCD include anti-depressants such as selective serotonin reuptake inhibitors (SSRIs) (e.g., paroxetine, sertraline, fluoxetine, escitalopram and fluvoxamine) and tricyclic antidepressants (e.g., clomipramine). Other non-limiting examples of therapeutic agents for OCD include riluzole, memantine, gabapentin, N-Acetylcysteine, lamotrigine, and atypical antipsychotics, such as olanzapine, quetiapine, and risperidone.

In some embodiments, treatment comprises behavioral therapy. Non-limiting examples of behavioral therapy include exposure and response prevention (ERP) and habit-reversal training.

In some embodiments, treatment comprises electroconvulsive therapy. In some embodiments, treatment comprises deep brain stimulation (DBS).

It is to be understood that any treatment described herein may be used alone or may be used in combination with any other treatment described herein.

In some embodiments, a subject identified as being at elevated risk of developing or having a neuropsychiatric disorder is treated. In some embodiments, the method comprises selecting a subject for treatment on the basis of the presence of one or more mutations as described herein. In some embodiments, the method comprises treating a subject with neuropsychiatric disorder characterized by the presence of one or more mutations as defined herein.

As used herein, “treat” or “treatment” includes, but is not limited to, preventing or reducing the development of a neuropsychiatric disorder or reducing or eliminating the symptoms of a neuropsychiatric.

An effective amount is a dosage of a therapy sufficient to provide a medically desirable result, such as treatment of a neuropsychiatric disorder. The effective amount will vary with the disorder to be treated, the age and physical condition of the subject being treated, the severity of the disorder, the duration of the treatment, the nature of any concurrent therapy, the specific route of administration and the like factors within the knowledge and expertise of the health practitioner.

Administration of a treatment may be accomplished by any method known in the art (see, e.g., Harrison's Principle of Internal Medicine, McGraw Hill Inc.). Administration may be local or systemic. Administration may be parenteral (e.g., intravenous, subcutaneous, or intradermal) or oral (e.g., sublingual or buccal). Compositions for different routes of administration are well known in the art (see, e.g., Remington's Pharmaceutical Sciences by E. W. Martin). Dosage will depend on the subject and the route of administration. Dosage can be determined by the skilled artisan.

### EXAMPLES

### Example 1

### Candidate Genes and Functional Noncoding Variants Identified in a Canine Model of Obsessive-Compulsive Disorder

### Methods: GWAS and Sequencing Region Selection

The GWAS that used the sample set described previously [14] was recalled using the MAGIC algorithm as described by Boyko et al. [15]. Briefly, MAGIC (Multidimensional Analysis for Genotype Intensity Clustering) does not use prior information to make genotype calls (i.e. cluster locations Hardy-Weinberg equilibrium, or complex normalization of probe intensities). Instead, it performs quantile normalization of the data for each chip independently followed by a Principal Component Analysis (PCA) of all chips on a SNP-by-SNP basis, neatly summarizing the raw data.

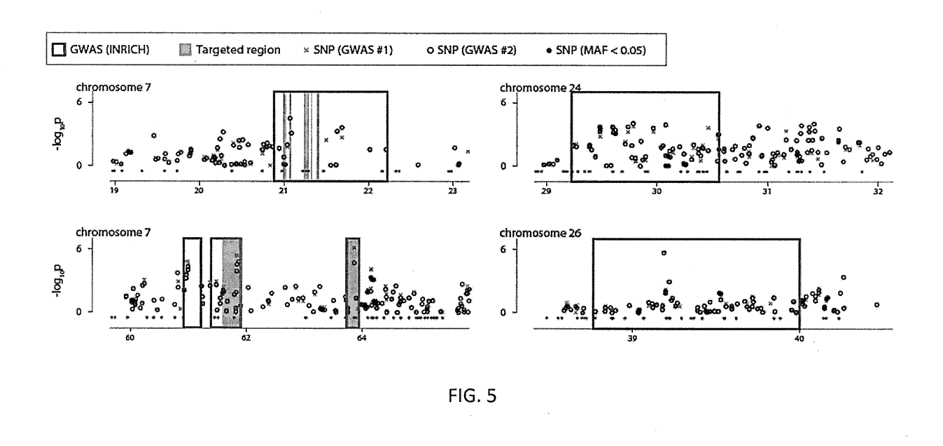

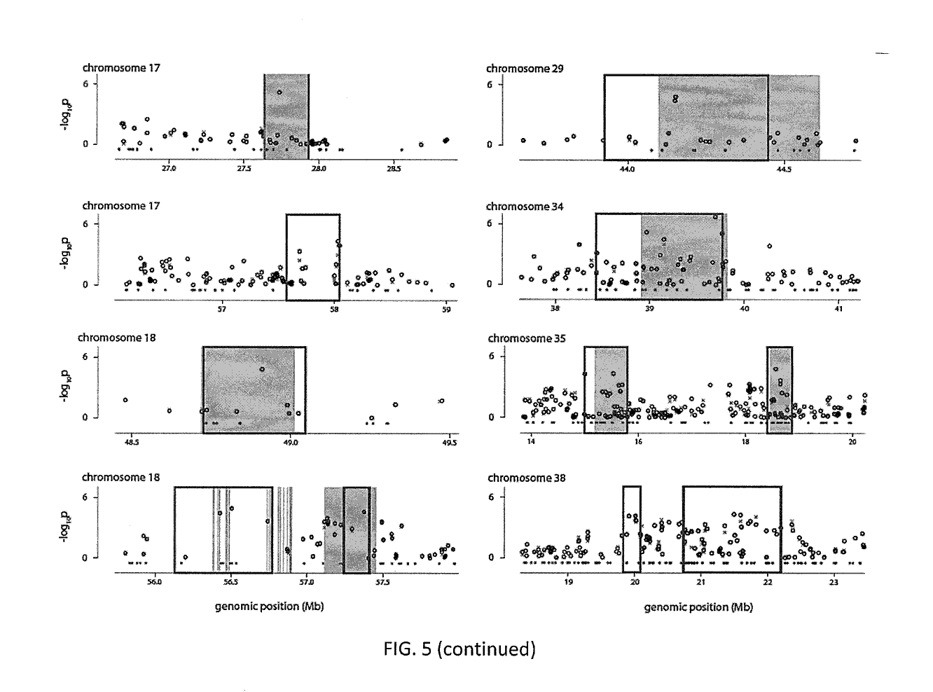

The processed data is then clustered into genotype calls through expectation maximization using a t-distribution mixture model. Association was calculated with standard chi-squared test in PLINK [59] (SNP genotype rate >90%, individual genotype rate >25%, minor allele frequency >5%) and regions were defined with LD-based clumping around SNPs with p<0.0001 (i.e. SNPs within 1 Mb with r2>0.8 and p<0.01) (Table 7 and FIG. 5). Regions of fixation were identified as regions of >1000 kb with more than five SNPs and >95% SNPs with MAF <0.05 and selected a subset found in breeds prone to OCD for targeted sequencing. From the associated and fixed regions a 5.8 Mb targeted sequencing array that optimized inclusion of potential genes of interest within the design limitations was designed (Table 8 and Table 9).

TABLE 7
Candidate associated regions identified in GWAS

| GWAS (new) | | GWAS (original) | | Top region (r2 > 0.8, with flanking genes) | | |
|---|---|---|---|---|---|---|
| 87 cases + 63 controls | | 92 cases + 68 controls | | | size | |
| SNP | p | SNP | p | region | (kb) | genes |
| chr7 63867472 | 2.1E−05 | chr7 | 7.6E−07 | chr7 | 213 | CDH2 |
| | | 63867472 | | 63741207-63954411 | | |
| chr7 61865715 | 1.6E−05 | chr7 | 3.8E−06 | chr7 | 507 | DSC3 |
| | | 61835240 | | 61392736-61900158 | | |
| chr34 39694895 | 1.9E−07 | | | chr34 | 1340 | TNIK, PLD1, TMEM212, |
| | | | | 38431782-39772222 | | FNDC3B, GHSR, TNFSF10 |
| chr26 39188777 | 2.1E−06 | | | chr26 | 1235 | DKK1, PRKG1 |
| | | | | 38766783-40001941 | | |
| chr17 27736094 | 6.1E−06 | | | chr17 | 292 | GALNT14, CAPN14 |
| | | | | 27638084-27929679 | | |
| chr18 56503107 | 1.1E−05 | | | chr18 | 644 | MARK2, C11orf84, |
| | | | | 56127466-56771285 | | C18H11orf84, C11orf95, |
| | | | | | | RTN3, ATL3, PLA2G16, |
| | | | | | | HRASLS2, RARRES3, |
| | | | | | | LGALS12, HRASLS5, |
| | | | | | | SLC22A9, SLC22A24, |
| | | | | | | SLC22A25, SLC22A10, |
| | | | | | | SLC22A8, SLC22A6 |
| chr29 44152594 | 1.5E−05 | | | chr29 | 523 | SDC2, PGCP, CPQ |
| | | | | 43925364-44448160 | | |
| chr18 48909564 | 1.6E−05 | | | chr18 | 321 | MOB2, DUSP8, KRTAP5- |
| | | | | 48725507-49046739 | | 11, KRTAP5-2, KRTAP5-9, |
| | | | | | | KRTAP5-8, KRTAP5-3, |
| | | | | | | KRTAP5-4, KRTAP5-7, |
| | | | | | | KRTAP5-6, IFITM10, CTSD |
| chr35 18565131 | 1.6E−05 | | | chr35 | 463 | GMPR, ATXN1 |
| | | | | 18409969-18872470 | | |
| chr18 57375179 | 2.4E−05 | | | chr18 | 168 | ASRGL1, SCGB1D2, |
| | | | | 57242322-57410796 | | SCGB1D1, SCGB2A1, |
| | | | | | | INCENP |
| chr7 21065761 | 3.0E−05 | | | chr7 | 1338 | EDEM3, FAM129A, RNF2, |
| | | | | 20881499-22219043 | | TRMT1L, SWT1, |
| | | | | | | IVNS1ABP, HMCN1 |
| chr17 58032126 | 4.7E−05 | | | chr17 | 474 | TRIM45, VTCN1, MAN1A2, |
| | | | | 57571016-58045329 | | FAM46C |
| chr38 21494582 | 4.7E−05 | | | chr38 | 1461 | LRRC52, RXRG, LMX1A, |
| | | | | 20737417-22198037 | | PBX1 |
| chr7 60990261 | 4.8E−05 | | | chr7 | 305 | TTR, DSG2, DSG3, DSG4, |
| | | | | 60919881-61225006 | | DSG1 |
| chr35 15535554 | 4.8E−05 | | | chr35 | 801 | PHACTR1, TBC1D7 |
| | | | | 14998490-15799243 | | |
| chr38 19918463 | 7.0E−05 | | | chr38 | 259 | TAF1A, MIA3, AIDA, |
| | | | | 19827135-20086439 | | BROX, FAM177B |
| chr24.29784557 | 8.7E−05 | | | chr24 | 1335 | |
| | | | | 29224804-30560165 | | |

TABLE 8
Sequencing array design, including GWAS regions and fixed regions that
overlap between the breeds predisposed with OCD.

| | | | | total | | |
|---|---|---|---|---|---|---|
| region | | size | targeting | target | fraction | |
| type | region | (kb) | strategy | (kb) | targeted | genes targeted |
| GWAS | chr7 | 235 | all | 235 | 1.00 | CDH2 |
| | 63733949-63968500 | | | | | |
| GWAS | chr7 | 318 | all | 318 | 1.00 | none |
| | 61598949-61916823 | | | | | |
| GWAS | chr34 | 902 | all | 902 | 1.00 | PLD1, TMEM212, FNDC3B, |
| | 38914699-39816833 | | | | | GHSR, TNFSF10 |
| GWAS | chr17 | 294 | all | 294 | 1.00 | GALNT14, CAPN14 |
| | 27631854-27926018 | | | | | |
| GWAS | chr18 | 515 | genes & | 24 | 0.05 | ATL3, PLA2G16, LGALS12, |
| | 56382749-56898210 | | conserved | | | HRASLS5, SLC22A8, SLC22A6, |
| | | | elements | | | CHRM1, SLC3A2, SNHG1, |
| | | | | | | SNORD30, SNORD31, |
| | | | | | | SNORD22, WDR74, STX5 |
| GWAS | chr29 | 512 | all | 512 | 1.00 | PGCP, CPQ, TSPYL5 |
| | 44099250-44611012 | | | | | |
| GWAS | chr18 | 292 | all | 292 | 1.00 | MOB2, DUSP8, KRTAP5-11, |
| | 48718949-49010600 | | | | | KRTAP5-2, KRTAP5-9, KRTAP5- |
| | | | | | | 8, KRTAP5-3, KRTAP5-4, |
| | | | | | | KRTAP5-7 |
| GWAS | chr35 | 417 | all | 417 | 1.00 | ATXN1 |
| | 18464094-18881027 | | | | | |
| GWAS | chr18 | 332 | all | 332 | 1.00 | AHNAK, SCGB1A1, ASRGL1, |
| | 57118970-57451000 | | | | | SCGB1D2, SCGB1D1, SCGB2A1, |
| | | | | | | INCENP |
| GWAS | chr7 | 419 | genes & | 20 | 0.05 | FAM129A, RNF2, TRMT1L, |
| | 20988159-21407321 | | conserved | | | SWT1, IVNS1ABP |
| | | | elements | | | |
| GWAS | chr35 | 605 | all | 605 | 1.00 | PHACTR1, TBC1D7 |
| | 15191960-15797327 | | | | | |
| FIXED | chr3 | 2764 | combined* | 1366 | 0.49 | EPB41L4A, NREP, STARD4, |
| | 3485979-6250329 | | | | | CAMK4, WDR36, TSLP, |
| | | | | | | SLC25A46, TMEM232, MAN2A1, |
| | | | | | | PJA2, FER |
| FIXED | chr3 | 1109 | genes & | 23 | 0.02 | PPP2R2C, MRFAP1, BLOC1S4, |
| | 61344672-62453220 | | conserved | | | CNO, KIAA0232, TBC1D14, |
| | | | elements | | | CCDC96, TADA2B, GRPEL1, |
| | | | | | | SORCS2 |
| FIXED | chr3 | 1356 | genes & | 120 | 0.09 | NOP14, C4orf10, MFSD10, ADD1, |
| | 64150954-65507021 | | conserved | | | SH3BP2, TNIP2, FAM193A, |
| | | | elements | | | RNF4, ZFYVE28, MXD4, HAUS3, |
| | | | | | | POLN, NAT8L, NELFA, WHSC2, |
| | | | | | | WHSC1, SCARNA22, LETM1, |
| | | | | | | FGFR3, TACC3, TMEM129, |
| | | | | | | SLBP, UVSSA, KIAA1530, MAEA |
| FIXED | chr17 | 889 | combined* | 148 | 0.17 | CTNNA2, LRRTM1 |
| | 46107254-46996631 | | | | | |
| FIXED | chr19 | 946 | combined* | 158 | 0.17 | none |
| | 12898854-13844607 | | | | | |

*see FIG. 5

TABLE 9
Targeted sequencing array design

| | | | | SIZE |
|---|---|---|---|---|
| CHR | START | END | TYPE | (KB) |
| 34 | 38914699 | 39816833 | GWAS | 902.134 |
| 35 | 15191960 | 15797327 | GWAS | 605.367 |
| 29 | 44099250 | 44611012 | GWAS | 511.762 |
| 35 | 18464094 | 18881027 | GWAS | 416.933 |
| 18 | 57118970 | 57451000 | GWAS | 332.03 |
| 7 | 61598949 | 61916823 | GWAS | 317.874 |
| 17 | 27631854 | 27926018 | GWAS | 294.164 |
| 18 | 48718949 | 49010600 | GWAS | 291.651 |
| 7 | 63733949 | 63968500 | GWAS | 234.551 |
| 18 | 56469261 | 56472729 | GWAS | 3.468 |
| 18 | 56887849 | 56890630 | GWAS | 2.781 |
| 7 | 21401554 | 21404020 | GWAS | 2.466 |
| 7 | 21242164 | 21244600 | GWAS | 2.436 |
| 7 | 21396050 | 21398329 | GWAS | 2.279 |
| 7 | 21405399 | 21407321 | GWAS | 1.922 |
| 18 | 56822053 | 56823731 | GWAS | 1.678 |
| 18 | 56896569 | 56898210 | GWAS | 1.641 |
| 7 | 21236971 | 21238333 | GWAS | 1.362 |
| 18 | 56766349 | 56767618 | GWAS | 1.269 |
| 7 | 21276449 | 21277631 | GWAS | 1.182 |
| 18 | 56385069 | 56386232 | GWAS | 1.163 |
| 18 | 56465860 | 56466939 | GWAS | 1.079 |
| 7 | 21007671 | 21008728 | GWAS | 1.057 |
| 18 | 56870850 | 56871900 | GWAS | 1.05 |
| 18 | 56810561 | 56811530 | GWAS | 0.969 |
| 18 | 56764362 | 56765216 | GWAS | 0.854 |
| 7 | 21394299 | 21395123 | GWAS | 0.824 |
| 18 | 56884754 | 56885435 | GWAS | 0.681 |
| 18 | 56845149 | 56845828 | GWAS | 0.679 |
| 18 | 56754353 | 56755020 | GWAS | 0.667 |
| 7 | 21257973 | 21258622 | GWAS | 0.649 |
| 18 | 56768452 | 56769100 | GWAS | 0.648 |
| 18 | 56891749 | 56892340 | GWAS | 0.591 |
| 18 | 56418151 | 56418731 | GWAS | 0.58 |
| 18 | 56382749 | 56383236 | GWAS | 0.487 |
| 7 | 21071954 | 21072437 | GWAS | 0.483 |
| 18 | 56808653 | 56809135 | GWAS | 0.482 |
| 7 | 21273754 | 21274225 | GWAS | 0.471 |
| 7 | 21069049 | 21069507 | GWAS | 0.458 |
| 18 | 56737363 | 56737819 | GWAS | 0.456 |
| 7 | 20994254 | 20994709 | GWAS | 0.455 |
| 18 | 56488149 | 56488600 | GWAS | 0.451 |
| 7 | 20997879 | 20998328 | GWAS | 0.449 |
| 7 | 20988159 | 20988600 | GWAS | 0.441 |
| 18 | 56411199 | 56411623 | GWAS | 0.424 |
| 7 | 21014449 | 21014833 | GWAS | 0.384 |
| 18 | 56763254 | 56763629 | GWAS | 0.375 |
| 7 | 21386461 | 21386831 | GWAS | 0.37 |
| 18 | 56486069 | 56486438 | GWAS | 0.369 |
| 7 | 21281152 | 21281520 | GWAS | 0.368 |
| 18 | 56748854 | 56749222 | GWAS | 0.368 |
| 7 | 21059461 | 21059826 | GWAS | 0.365 |
| 7 | 21234149 | 21234513 | GWAS | 0.364 |
| 18 | 56425754 | 56426115 | GWAS | 0.361 |
| 7 | 21263454 | 21263800 | GWAS | 0.346 |
| 7 | 21320254 | 21320600 | GWAS | 0.346 |
| 7 | 20989963 | 20990300 | GWAS | 0.337 |
| 3 | 3624353 | 4029207 | fixed | 404.854 |
| 3 | 5942854 | 6250329 | fixed | 307.475 |
| 3 | 4800199 | 5084936 | fixed | 284.737 |
| 3 | 4039899 | 4270130 | fixed | 230.231 |
| 19 | 13514749 | 13656717 | fixed | 141.968 |
| 17 | 46660152 | 46762926 | fixed | 102.774 |
| 3 | 4593549 | 4695524 | fixed | 101.975 |
| 17 | 46457659 | 46478917 | fixed | 21.258 |
| 19 | 12898854 | 12906100 | fixed | 7.246 |
| 3 | 65129750 | 65135529 | fixed | 5.779 |
| 3 | 64154154 | 64159336 | fixed | 5.182 |
| 3 | 64187752 | 64192629 | fixed | 4.877 |
| 3 | 61779950 | 61783514 | fixed | 3.564 |
| 19 | 13691849 | 13695100 | fixed | 3.251 |
| 3 | 64987199 | 64990231 | fixed | 3.032 |
| 17 | 46107254 | 46110131 | fixed | 2.877 |
| 3 | 65118699 | 65121341 | fixed | 2.642 |
| 17 | 46899874 | 46902426 | fixed | 2.552 |
| 3 | 65494150 | 65496437 | fixed | 2.287 |
| 3 | 64275154 | 64277432 | fixed | 2.278 |
| 3 | 65003959 | 65006137 | fixed | 2.178 |
| 3 | 64160151 | 64162235 | fixed | 2.084 |
| 3 | 4368049 | 4370100 | fixed | 2.051 |
| 3 | 65101749 | 65103728 | fixed | 1.979 |
| 3 | 65187899 | 65189826 | fixed | 1.927 |
| 3 | 64737074 | 64739000 | fixed | 1.926 |
| 3 | 61938584 | 61940505 | fixed | 1.921 |
| 3 | 65473753 | 65475637 | fixed | 1.884 |
| 3 | 64280654 | 64282535 | fixed | 1.881 |
| 3 | 64394264 | 64396030 | fixed | 1.766 |
| 3 | 64767471 | 64769212 | fixed | 1.741 |
| 3 | 64980049 | 64981740 | fixed | 1.691 |
| 3 | 65476749 | 65478423 | fixed | 1.674 |
| 3 | 65505349 | 65507021 | fixed | 1.672 |
| 3 | 64543459 | 64545130 | fixed | 1.671 |
| 3 | 64385051 | 64386719 | fixed | 1.668 |
| 3 | 65213174 | 65214819 | fixed | 1.645 |
| 3 | 64997179 | 64998811 | fixed | 1.632 |
| 3 | 4356282 | 4357900 | fixed | 1.618 |
| 3 | 5681049 | 5682612 | fixed | 1.563 |
| 3 | 65158161 | 65159721 | fixed | 1.56 |
| 3 | 65001570 | 65003127 | fixed | 1.557 |
| 3 | 64197554 | 64199027 | fixed | 1.473 |
| 17 | 46538449 | 46539835 | fixed | 1.386 |
| 3 | 64587063 | 64588421 | fixed | 1.358 |
| 3 | 61896649 | 61898000 | fixed | 1.351 |
| 3 | 65007753 | 65009100 | fixed | 1.347 |
| 3 | 65031054 | 65032400 | fixed | 1.346 |
| 3 | 64436681 | 64438000 | fixed | 1.319 |
| 3 | 64173464 | 64174733 | fixed | 1.269 |
| 3 | 64351454 | 64352716 | fixed | 1.262 |
| 3 | 3536249 | 3537500 | fixed | 1.251 |
| 3 | 5470879 | 5472121 | fixed | 1.242 |
| 17 | 46657664 | 46658836 | fixed | 1.172 |
| 3 | 64597554 | 64598722 | fixed | 1.168 |
| 3 | 61900549 | 61901712 | fixed | 1.163 |
| 3 | 64926152 | 64927310 | fixed | 1.158 |
| 3 | 64710161 | 64711300 | fixed | 1.139 |
| 3 | 64179753 | 64180841 | fixed | 1.088 |
| 3 | 65013049 | 65014135 | fixed | 1.086 |
| 3 | 5227459 | 5228534 | fixed | 1.075 |
| 3 | 64363460 | 64364519 | fixed | 1.059 |
| 3 | 64831649 | 64832704 | fixed | 1.055 |
| 3 | 64381849 | 64382900 | fixed | 1.051 |
| 17 | 46578470 | 46579514 | fixed | 1.044 |
| 3 | 3500174 | 3501132 | fixed | 0.958 |
| 3 | 61823363 | 61824300 | fixed | 0.937 |
| 3 | 64169564 | 64170500 | fixed | 0.936 |
| 3 | 64164299 | 64165214 | fixed | 0.915 |
| 3 | 5676349 | 5677232 | fixed | 0.883 |
| 3 | 64823451 | 64824329 | fixed | 0.878 |
| 3 | 65358549 | 65359424 | fixed | 0.875 |
| 3 | 65486559 | 65487434 | fixed | 0.875 |
| 3 | 4589561 | 4590435 | fixed | 0.874 |
| 17 | 46896850 | 46897722 | fixed | 0.872 |
| 17 | 46448254 | 46449123 | fixed | 0.869 |
| 3 | 65112561 | 65113404 | fixed | 0.843 |
| 3 | 64278559 | 64279400 | fixed | 0.841 |
| 19 | 13696284 | 13697114 | fixed | 0.83 |
| 3 | 64412053 | 64412833 | fixed | 0.78 |
| 3 | 65484959 | 65485728 | fixed | 0.769 |
| 17 | 46269854 | 46270622 | fixed | 0.768 |
| 17 | 46607349 | 46608100 | fixed | 0.751 |
| 17 | 46790949 | 46791700 | fixed | 0.751 |
| 3 | 5559262 | 5560005 | fixed | 0.743 |
| 3 | 64200669 | 64201412 | fixed | 0.743 |
| 3 | 5177062 | 5177800 | fixed | 0.738 |
| 3 | 65046564 | 65047300 | fixed | 0.736 |
| 3 | 61796051 | 61796736 | fixed | 0.685 |
| 3 | 61580050 | 61580732 | fixed | 0.682 |
| 17 | 46995949 | 46996631 | fixed | 0.682 |
| 3 | 61944552 | 61945228 | fixed | 0.676 |
| 3 | 65458254 | 65458928 | fixed | 0.674 |
| 3 | 64409259 | 64409932 | fixed | 0.673 |
| 3 | 64928054 | 64928727 | fixed | 0.673 |
| 17 | 46974154 | 46974827 | fixed | 0.673 |
| 3 | 65447949 | 65448619 | fixed | 0.67 |
| 3 | 4340753 | 4341420 | fixed | 0.667 |
| 3 | 65327449 | 65328109 | fixed | 0.66 |
| 3 | 65289059 | 65289709 | fixed | 0.65 |
| 3 | 64982563 | 64983211 | fixed | 0.648 |
| 17 | 46277699 | 46278300 | fixed | 0.601 |
| 19 | 13326599 | 13327200 | fixed | 0.601 |
| 3 | 65491850 | 65492437 | fixed | 0.587 |
| 3 | 61912849 | 61913433 | fixed | 0.584 |
| 17 | 46644954 | 46645535 | fixed | 0.581 |
| 17 | 46236451 | 46237031 | fixed | 0.58 |
| 3 | 61746051 | 61746627 | fixed | 0.576 |
| 3 | 5640153 | 5640728 | fixed | 0.575 |
| 19 | 13445451 | 13446024 | fixed | 0.573 |
| 19 | 13834251 | 13834824 | fixed | 0.573 |
| 3 | 64433260 | 64433829 | fixed | 0.569 |
| 3 | 61344672 | 61345234 | fixed | 0.562 |
| 3 | 61925553 | 61926115 | fixed | 0.562 |
| 19 | 13070051 | 13070613 | fixed | 0.562 |
| 3 | 61642472 | 61643033 | fixed | 0.561 |
| 3 | 65325954 | 65326506 | fixed | 0.552 |
| 3 | 64770749 | 64771300 | fixed | 0.551 |
| 3 | 65203953 | 65204504 | fixed | 0.551 |
| 3 | 65323364 | 65323914 | fixed | 0.55 |
| 3 | 64392551 | 64393100 | fixed | 0.549 |
| 3 | 65099872 | 65100419 | fixed | 0.547 |
| 17 | 46568061 | 46568608 | fixed | 0.547 |
| 3 | 65350564 | 65351109 | fixed | 0.545 |
| 17 | 46924179 | 46924723 | fixed | 0.544 |
| 17 | 46961669 | 46962212 | fixed | 0.543 |
| 3 | 5548481 | 5549022 | fixed | 0.541 |
| 3 | 64177995 | 64178534 | fixed | 0.539 |
| 3 | 61556699 | 61557200 | fixed | 0.501 |
| 17 | 46819650 | 46820141 | fixed | 0.491 |
| 3 | 64283550 | 64284037 | fixed | 0.487 |
| 3 | 65020049 | 65020536 | fixed | 0.487 |
| 3 | 5243149 | 5243635 | fixed | 0.486 |
| 3 | 64975649 | 64976135 | fixed | 0.486 |
| 17 | 46583049 | 46583534 | fixed | 0.485 |
| 3 | 5225549 | 5226033 | fixed | 0.484 |
| 17 | 46855151 | 46855635 | fixed | 0.484 |
| 3 | 3494351 | 3494834 | fixed | 0.483 |
| 3 | 5280349 | 5280832 | fixed | 0.483 |
| 3 | 64757551 | 64758034 | fixed | 0.483 |
| 3 | 4343553 | 4344035 | fixed | 0.482 |
| 3 | 64403951 | 64404433 | fixed | 0.482 |
| 17 | 46872152 | 46872634 | fixed | 0.482 |
| 3 | 5345550 | 5346030 | fixed | 0.48 |
| 3 | 64681752 | 64682232 | fixed | 0.48 |
| 3 | 64704254 | 64704734 | fixed | 0.48 |
| 3 | 5637754 | 5638233 | fixed | 0.479 |
| 3 | 5649554 | 5650031 | fixed | 0.477 |
| 3 | 61391852 | 61392328 | fixed | 0.476 |
| 3 | 64182852 | 64183328 | fixed | 0.476 |
| 3 | 65373251 | 65373727 | fixed | 0.476 |
| 3 | 5207750 | 5208223 | fixed | 0.473 |
| 3 | 64284852 | 64285325 | fixed | 0.473 |
| 3 | 64977060 | 64977533 | fixed | 0.473 |
| 3 | 3518752 | 3519224 | fixed | 0.472 |
| 3 | 5359764 | 5360236 | fixed | 0.472 |
| 3 | 65210364 | 65210836 | fixed | 0.472 |
| 17 | 46613351 | 46613823 | fixed | 0.472 |
| 3 | 5773349 | 5773820 | fixed | 0.471 |
| 3 | 61890053 | 61890524 | fixed | 0.471 |
| 3 | 65481052 | 65481523 | fixed | 0.471 |
| 17 | 46800159 | 46800629 | fixed | 0.47 |
| 3 | 3495562 | 3496031 | fixed | 0.469 |
| 3 | 5857761 | 5858229 | fixed | 0.468 |
| 3 | 64218252 | 64218719 | fixed | 0.467 |
| 3 | 5165064 | 5165530 | fixed | 0.466 |
| 3 | 5304549 | 5305015 | fixed | 0.466 |
| 19 | 13680154 | 13680620 | fixed | 0.466 |
| 3 | 61552354 | 61552819 | fixed | 0.465 |
| 3 | 65200450 | 65200914 | fixed | 0.464 |
| 3 | 65343754 | 65344218 | fixed | 0.464 |
| 17 | 46626649 | 46627113 | fixed | 0.464 |
| 3 | 5903654 | 5904116 | fixed | 0.462 |
| 19 | 13710753 | 13711215 | fixed | 0.462 |
| 3 | 64168051 | 64168512 | fixed | 0.461 |
| 17 | 46547854 | 46548314 | fixed | 0.46 |
| 19 | 13267059 | 13267519 | fixed | 0.46 |
| 3 | 64462951 | 64463409 | fixed | 0.458 |
| 3 | 5408473 | 5408929 | fixed | 0.456 |
| 3 | 5315864 | 5316318 | fixed | 0.454 |
| 3 | 64781249 | 64781703 | fixed | 0.454 |
| 3 | 65117354 | 65117806 | fixed | 0.452 |
| 3 | 64862549 | 64863000 | fixed | 0.451 |
| 19 | 13287449 | 13287900 | fixed | 0.451 |
| 3 | 4393763 | 4394211 | fixed | 0.448 |
| 3 | 5403653 | 5404100 | fixed | 0.447 |
| 3 | 61367984 | 61368431 | fixed | 0.447 |
| 3 | 64206653 | 64207100 | fixed | 0.447 |
| 3 | 64692260 | 64692707 | fixed | 0.447 |
| 3 | 64930853 | 64931300 | fixed | 0.447 |
| 3 | 4337554 | 4338000 | fixed | 0.446 |
| 3 | 61764354 | 61764800 | fixed | 0.446 |
| 3 | 64758794 | 64759240 | fixed | 0.446 |
| 3 | 5318759 | 5319200 | fixed | 0.441 |
| 3 | 65148959 | 65149400 | fixed | 0.441 |
| 3 | 5301660 | 5302100 | fixed | 0.44 |
| 3 | 65022999 | 65023438 | fixed | 0.439 |
| 3 | 64541899 | 64542337 | fixed | 0.438 |
| 3 | 5667099 | 5667533 | fixed | 0.434 |
| 3 | 61384269 | 61384701 | fixed | 0.432 |
| 3 | 5327670 | 5328100 | fixed | 0.43 |
| 3 | 5661899 | 5662327 | fixed | 0.428 |
| 3 | 64779199 | 64779626 | fixed | 0.427 |
| 17 | 46850683 | 46851105 | fixed | 0.422 |
| 3 | 4374799 | 4375219 | fixed | 0.42 |
| 3 | 5753099 | 5753517 | fixed | 0.418 |
| 3 | 64213299 | 64213708 | fixed | 0.409 |
| 3 | 65043899 | 65044300 | fixed | 0.401 |
| 3 | 61788951 | 61789339 | fixed | 0.388 |
| 3 | 65472051 | 65472435 | fixed | 0.384 |
| 3 | 65483649 | 65484033 | fixed | 0.384 |
| 3 | 61777850 | 61778232 | fixed | 0.382 |
| 3 | 65000250 | 65000631 | fixed | 0.381 |
| 3 | 64354652 | 64355032 | fixed | 0.38 |
| 3 | 64421654 | 64422034 | fixed | 0.38 |
| 3 | 64914249 | 64914628 | fixed | 0.379 |
| 3 | 65017651 | 65018028 | fixed | 0.377 |
| 3 | 3544254 | 3544629 | fixed | 0.375 |
| 3 | 61859564 | 61859939 | fixed | 0.375 |
| 3 | 61892254 | 61892629 | fixed | 0.375 |
| 17 | 46876449 | 46876824 | fixed | 0.375 |
| 3 | 61886350 | 61886724 | fixed | 0.374 |
| 3 | 62438564 | 62438938 | fixed | 0.374 |
| 3 | 4345949 | 4346321 | fixed | 0.372 |
| 3 | 4380250 | 4380622 | fixed | 0.372 |
| 3 | 64755951 | 64756323 | fixed | 0.372 |
| 3 | 4366752 | 4367123 | fixed | 0.371 |
| 3 | 62452849 | 62453220 | fixed | 0.371 |
| 3 | 64556961 | 64557332 | fixed | 0.371 |
| 17 | 46808849 | 46809220 | fixed | 0.371 |
| 3 | 64601763 | 64602133 | fixed | 0.37 |
| 3 | 61357859 | 61358228 | fixed | 0.369 |
| 3 | 61389052 | 61389421 | fixed | 0.369 |
| 3 | 64690270 | 64690639 | fixed | 0.369 |
| 3 | 3572459 | 3572827 | fixed | 0.368 |
| 3 | 61888659 | 61889027 | fixed | 0.368 |
| 3 | 64559053 | 64559421 | fixed | 0.368 |
| 3 | 4353349 | 4353716 | fixed | 0.367 |
| 3 | 5400551 | 5400918 | fixed | 0.367 |
| 3 | 61776649 | 61777016 | fixed | 0.367 |
| 3 | 4376354 | 4376719 | fixed | 0.365 |
| 3 | 5679059 | 5679424 | fixed | 0.365 |
| 3 | 64911473 | 64911836 | fixed | 0.363 |
| 3 | 64150954 | 64151316 | fixed | 0.362 |
| 3 | 64427049 | 64427408 | fixed | 0.359 |
| 3 | 64746459 | 64746818 | fixed | 0.359 |
| 19 | 13049264 | 13049623 | fixed | 0.359 |
| 3 | 65164949 | 65165307 | fixed | 0.358 |
| 17 | 46781173 | 46781531 | fixed | 0.358 |
| 3 | 4372864 | 4373221 | fixed | 0.357 |
| 3 | 62221471 | 62221828 | fixed | 0.357 |
| 3 | 65216154 | 65216511 | fixed | 0.357 |
| 3 | 61394053 | 61394409 | fixed | 0.356 |
| 3 | 64413973 | 64414328 | fixed | 0.355 |
| 3 | 56197650 | 56198004 | fixed | 0.354 |
| 3 | 3512864 | 3513217 | fixed | 0.353 |
| 3 | 64166354 | 64166707 | fixed | 0.353 |
| 3 | 3485979 | 3486331 | fixed | 0.352 |
| 19 | 13797554 | 13797906 | fixed | 0.352 |
| 3 | 5362549 | 5362900 | fixed | 0.351 |
| 3 | 5754471 | 5754822 | fixed | 0.351 |
| 17 | 46617150 | 46617500 | fixed | 0.35 |
| 3 | 4592551 | 4592900 | fixed | 0.349 |
| 3 | 64750451 | 64750800 | fixed | 0.349 |
| 3 | 3552179 | 3552526 | fixed | 0.347 |
| 3 | 3568553 | 3568900 | fixed | 0.347 |
| 3 | 5245853 | 5246200 | fixed | 0.347 |
| 3 | 5229654 | 5230000 | fixed | 0.346 |
| 3 | 62315489 | 62315835 | fixed | 0.346 |
| 3 | 65277054 | 65277400 | fixed | 0.346 |
| 3 | 5235759 | 5236101 | fixed | 0.342 |
| 3 | 3498362 | 3498703 | fixed | 0.341 |
| 3 | 64978383 | 64978723 | fixed | 0.34 |
| 3 | 64735070 | 64735409 | fixed | 0.339 |
| 3 | 61798399 | 61798734 | fixed | 0.335 |
| 3 | 65110699 | 65111033 | fixed | 0.334 |
| 3 | 5311489 | 5311822 | fixed | 0.333 |
| 3 | 61904474 | 61904807 | fixed | 0.333 |
| 3 | 65128491 | 65128824 | fixed | 0.333 |
| 3 | 5901169 | 5901500 | fixed | 0.331 |
| 3 | 65016269 | 65016600 | fixed | 0.331 |
| 3 | 64373284 | 64373600 | fixed | 0.316 |
| 19 | 13844352 | 13844607 | fixed | 0.255 |
| 3 | 61916864 | 61917100 | fixed | 0.236 |

Gene Set Enrichment Analysis

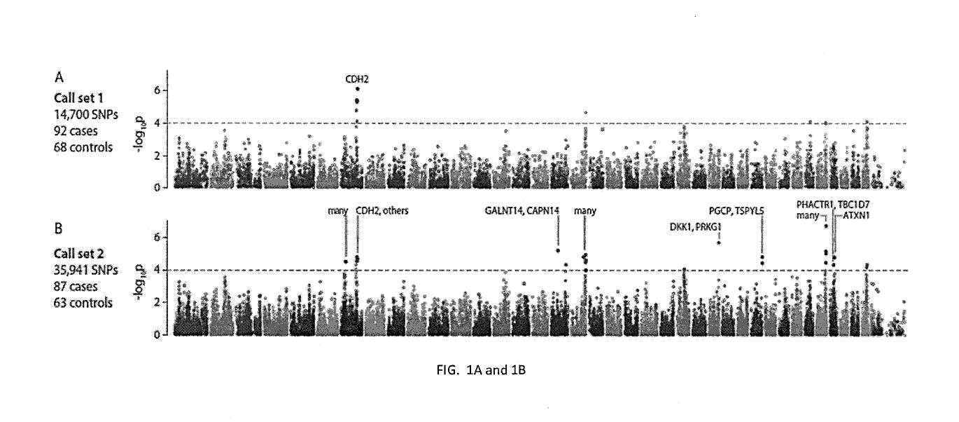

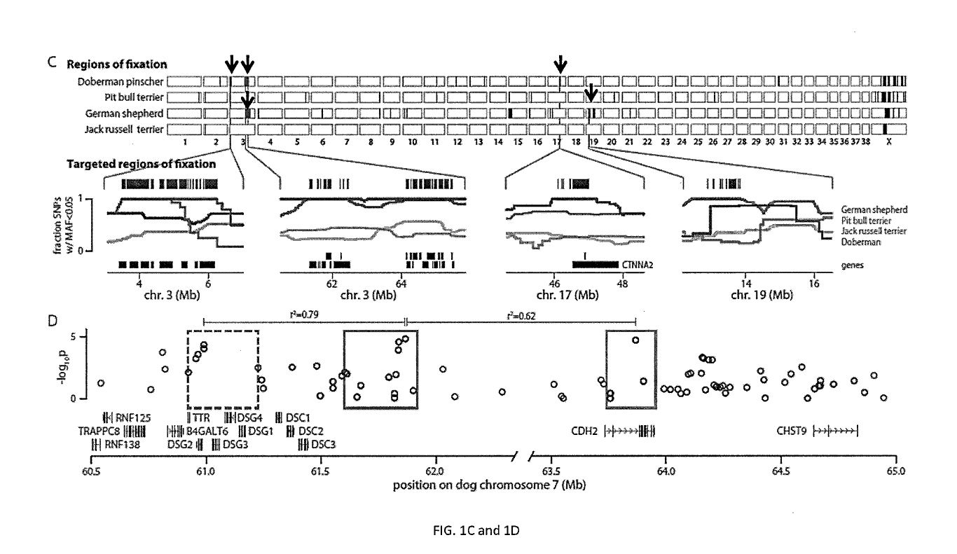

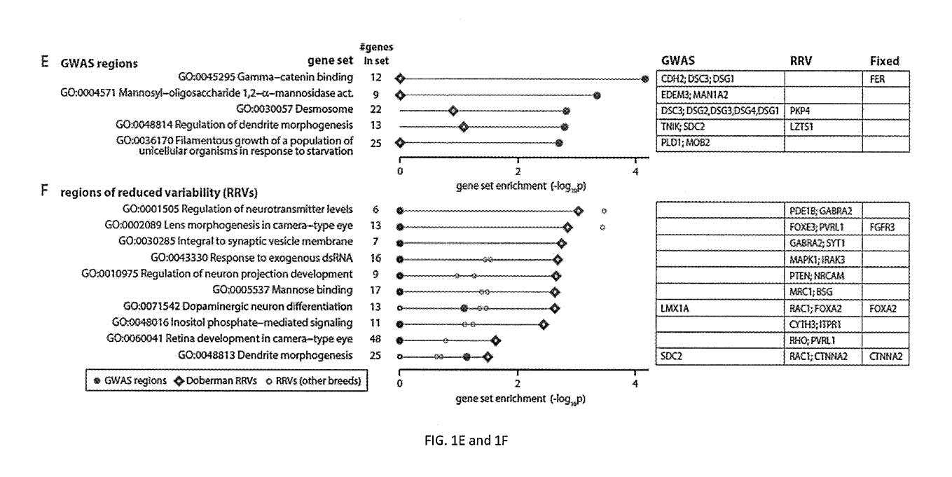

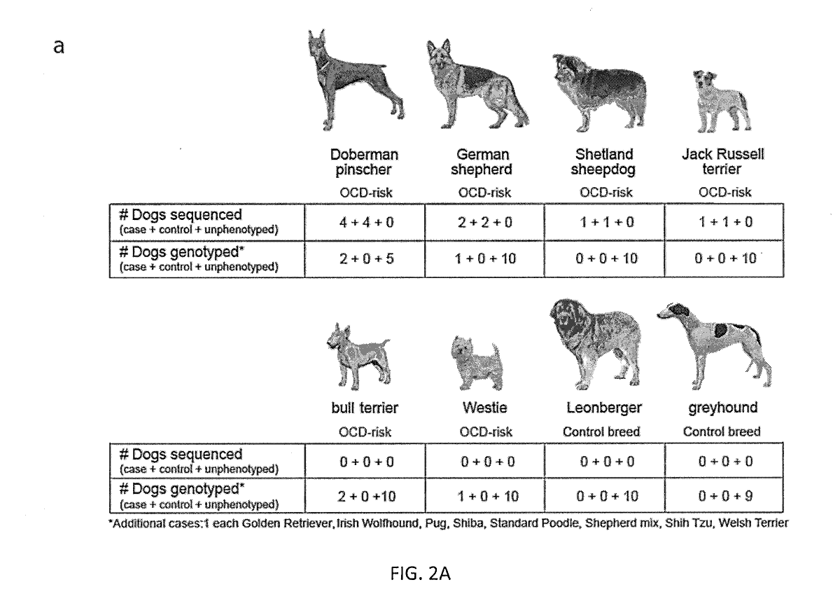

The GWAS regions were expanded to include all genes within 500 kb of the original region start or end (Table 7). Regions of reduced relative variability (RRVs) were defined by comparing the DP breed to 24 other dog breeds from a published reference dataset and identifying the 1% least variable 150 kb regions in DP [23]. INRICH was run with 1,000,000 permutations to test regions for enrichment in any gene sets from the gene ontology catalog. All gene sets with between 5 and 1000 genes (downloaded from www.geneontology.org) were tested [17]. A map file of 16,433 genes lifted over to canFam2.0 from the hg19 RefSeq Gene catalog (UCSC Genome Brower, single match using default parameters) was used [60]. To identify gene sets with unusually high enrichment in the DP RRVs for all sets with p<0.05 and at least 2 RRV genes in DP, the average difference in enrichment p values between DP and 24 other breeds [26] were calculated (FIG. 1F).

Sequenced Samples

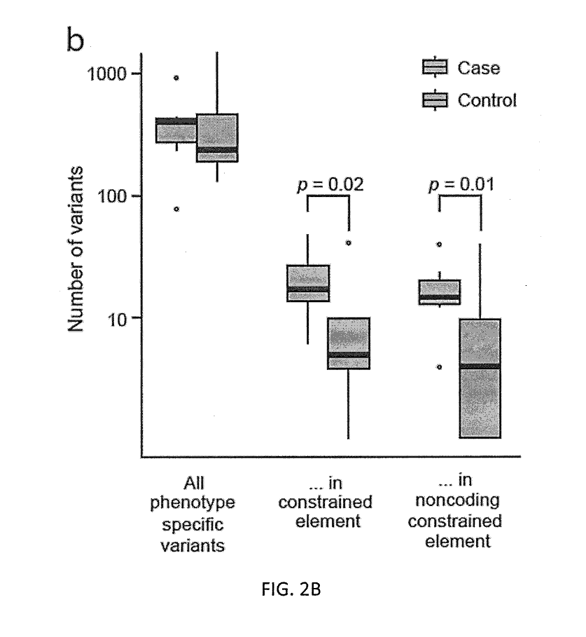

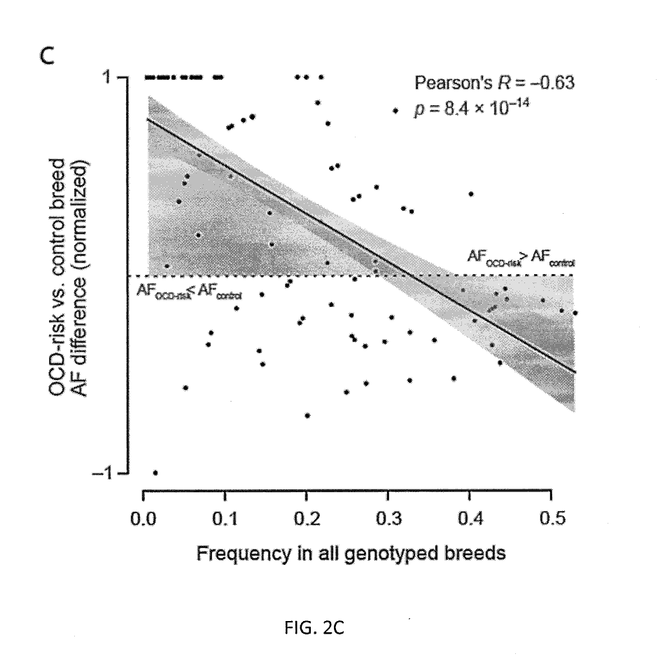

The targeted sequencing experiment comprised a total of eight cases and eight controls from multiple breeds: DP (4 cases+4 controls), German shepherd (2 cases+2 controls), Jack Russell terrier (Jack Russell terriers; 1 case+1 control) and Shetland sheepdog (Shetland sheepdogs; 1 case+1 control; FIG. 2A). The four DP cases were from the GWA-study [14].

Targeted Sequencing and Variant Calling

The 16 samples were individually barcoded and the targeted regions were captured by NimbleGen Sequence Capture 385 K Array according to the manufacturer's protocol. The captured samples were then pooled and sequenced on Illumina Genome Analyzer II. Paired-end 76-bp reads were aligned to canFam2.0 and PCR duplicates were removed using Picard [61], and realignment and recalibration were processed through Genome Analysis Toolkit (GATK) [62,63]. SNPs and small INDELs were identified using GATK. The variants that pass the GATK standard filters only were considered. Larger structural variants were detected by GenomeSTRiP [64]. The alignments were checked of all discovered deletion sites for aberrant read-pairs and read-depth using Integrative Genomics Viewer [65] to ensure the reliability of the calls. A Shetland sheepdog pair was excluded where the control has lower SNP accuracy.

Genotyping Candidate Sequence Variants

Case-specific variants were selected that were within evolutionarily constrained elements determined by 29 mammals sequence dataset [27]. A subset of the variants meeting one of the following criteria was then selected: (i) case-only variants within DP breed; (ii) case-only variants within CDH2, PGCP, CTNNA2 and ATXN1 that are identified by gene-based analysis; (iii) case-only variants across at least two breeds; (iv) potential functional variants annotated as nonsense, splicing or missense (predicted to be “probably” or “possibly damaging” by Polyphen-2 [66]) and case-only variants in at least one breed; (v) variants within CDH2 risk haplotype; and (vi) top associated variants from GWA-analysis. Of 140 variants that met one of the criteria, 127 variants passed Sequenom design standards, and were genotyped using the Sequenom iPlex system. An independent set of 94 dogs was employed that consisted of ten dogs without obvious health problems for each of six OCD-risk breeds (i.e. four sequenced breeds and West Highland white terrier [Westie] and bull terrier [bull terrier]) and two control breeds without known psychiatric problems (greyhound and Leonberger), and fourteen additional OCD cases from various breeds (2 bull terrier, 2 DP, 1 German shepherd, 1 Westie, 1 Golden retriever, 1 Irish wolfhound, 1 pug, 1 Shiba, 1 Shepherd mix, 1 standard poodle, 1 Shih Tzu, 1 Welsh terrier). Genotype data were cleaned by removing samples with missing genotype rates >10% and excluding SNPs with call rates <90%. After the quality control, 114 SNPs and 88 dogs (19 [10 Leonbergers+9 Greyhounds] from control breeds and 69 [14 cases+5 DP+10 bull terrier+10 Westie+10 German shepherd+10 Shetland sheepdogs+10 Jack Russell terriers] from OCD-risk breeds or cases) were retained in the analysis (FIG. 2A and Table 8).

Gene-Based Analysis

Each gene region was defined using the coordinates from RefSeq hg19 lifted over to CanFam2.0 plus 5 kb flanking sequence on each side. The number of case- and control-only variants was counted and compared the counts for each gene. Genes that have excessive case-only variants relative to control-only variants were considered as potential risk genes for OCD. The same analysis was applied to the variants within constrained elements. To correct for gene size, the ratio of the number of case-only variants and the number of control-only variants for each gene additionally was calculated.

Electrophoretic Mobility Shift Assay (EMSA)

For each allele of the tested SNPs in a regulatory region between CDH2 and DSC3, pairs of 5′-biotinylated oligonucleotides were obtained from IDT Inc (Coralville, Iowa, USA; Table 10 (SEQ ID NOs:1-16)). Equal volumes of forward and reverse oligos (100 μM) were mixed and heated at 95° C. for 5 minutes and then cooled to room temperature. 50 fmol annealed probes were incubated at room temperature for 30 minutes with 10 mg SK-N-BE (2) nuclear extract (Active Motif). The remaining steps followed the LightShift Chemiluminescent EMSA Kit protocol (Thermo Scientific).

TABLE 10
Oligonucleotide sequences (human) used for EMSA
(SEQ ID NOs: 1-16).

| | |
|---|---|
| BIO-835.w.T.hu.F | BIO-ACCTGCACCAACTAATTAGGGTTCAAACC |
| | (SEQ ID NO: 1) |
| BIO-835.w.T.hu.R | BIO-GGTTTGAACCCTAATTAGTTGGTGCAGGT |
| | (SEQ ID NO: 2) |
| BIO-835.m.C.hu.F | BIO-ACCTGCACCAACTAGTTAGGGTTCAAACC |
| | (SEQ ID NO: 3) |
| BIO-835.m.C.hu.R | BIO-GGTTTGAACCCTAACTAGTTGGTGCAGGT |
| | (SEQ ID NO: 4) |
| BIO-855.w.A.hu.F | BIO-TCGCTGGGTGGCTCATGGACCTGCA |
| | (SEQ ID NO: 5) |
| BIO-855.w.A.hu.R | BIO-TGCAGGTCCATGAGCCACCCAGCGA |
| | (SEQ ID NO: 6) |
| BIO-855.m.T.hu.F | BIO-TCGCTGGGTGGCACATGGACCTGCA |
| | (SEQ ID NO: 7) |
| BIO-855.m.T.hu.R | BIO-TGCAGGTCCATGTGCCACCCAGCGA |
| | (SEQ ID NO: 8) |
| 835.w.T.hu.F | ACCTGCACCAACTAATTAGGGTTCAAACC |
| | (SEQ ID NO: 9) |
| 835.w.T.hu.R | GGTTTGAACCCTAATTAGTTGGTGCAGGT |
| | (SEQ ID NO: 10) |
| 835.m.C.hu.F | ACCTGCACCAACTAGTTAGGGTTCAAACC |
| | (SEQ ID NO: 11) |
| 835.m.C.hu.R | GGTTTGAACCCTAACTAGTTGGTGCAGGT |
| | (SEQ ID NO: 12) |
| 855.w.A.hu.F | TCGCTGGGTGGCTCATGGACCTGCA |
| | (SEQ ID NO: 13) |
| 855.w.A.hu.R | TGCAGGTCCATGAGCCACCCAGCGA |
| | (SEQ ID NO: 14) |
| 855.m.T.hu.F | TCGCTGGGTGGCACATGGACCTGCA |
| | (SEQ ID NO: 15) |
| 855.m.T.hu.R | TGCAGGTCCATGTGCCACCCAGCGA |
| | (SEQ ID NO: 16) |

Luciferase Reporter Assay

The activity of a putative regulatory element and the effect of SNP35 and SNP55 on gene expression were examined by luciferase reporter assay. 879 bp-long orthologous sequence spanning SNP35 and SNP55 was PCR amplified from human DNA samples (Table 11 (SEQ ID NOs: 17-26)). The risk alleles were introduced by site-directed mutagenesis kit. The wild type and mutant DNA fragments were cloned into a firefly luciferase reporter plasmid (pGL4.23 Promega). The test constructs were transiently co-transfected with a Renilla luciferase reporter plasmid (pGL4.73, Promega) as an internal control into neuroblastoma SK-N-BE (2) cells. All constructs were tested in triplicates and repeated three times in a double-blinded manner.

TABLE 11
Oligonucleotide sequences (human) used for
luciferase assay (SEQ ID NOs: 17-26).

| | |
|---|---|
| enhancer_F | TTTGCTTTCTAACAATGAAACCAC (SEQ ID NO: 17) |
| enhancer_R | TTGGCACAATTTACCGGTTT (SEQ ID NO: 18) |
| negative_F | GAGTTGGGTGTAGGGGTCAA (SEQ ID NO: 19) |
| negative_R | GGTTTTGAACCTCCGCAATA (SEQ ID NO: 20) |
| enhancer_ | TGGACCTGCACCAACTAGTTAGGGTTCAAACCAAC |
| mut835_F | (SEQ ID NO: 21) |
| enhancer_ | GTTGGTTTGAACCCTAACTAGTTGGTGCAGGTCCA |
| mut835_R | (SEQ ID NO: 22) |
| enhancer_ | CTCGCTGGGTGGCACATGGACCTGCAC (SEQ ID NO: |
| mut855_F | 23) |
| enhancer_ | GTGCAGGTCCATGTGCCACCCAGCGAG (SEQ ID NO: |
| mut855_R | 24) |
| vector_F | CTAGCAAAATAGGCTGTCCC (SEQ ID NO: 25) |
| vector_R | GACGATAGTCATGCCCCGCG (SEQ ID NO: 26) |

Cell Cultures

Human SK-N-BE (2) cells were purchased from ATCC. The cells were maintained at 37° C. and 5% CO2 in 1:1 mixture of ATCC-formulated Eagle's Minimum Essential Medium (EMEM) and F-12 K Medium supplemented with 10% fetal bovine serum, 100 units/ml penicillin and 100 ug/ml streptomycin.

Real-Time qPCR

Real-time qPCR was performed using Quantifast SYBR Green PCR kit (Qiagen) on Lightcycler 480 system (Roche Applied Science). The reaction volumes were adjusted to 10 ul with 3 ul of DNA (10 ng), 1 ul of both primers (10 uM) and 5 ul of Master Mix. The qPCR program was as follows: pre-incubation at 95° C. for 5 minutes, followed by 40 cycles of two-step amplification (10 seconds at 95° C., 1 minute at 60° C.). All the experiments were carried out in triplicates and include negative control without DNA. The primer sets used to detect PGCP deletion is shown in Table 12 (SEQ ID NOs: 27-34) below.

TABLE 12
The primer sets inside and outside the PGCP dele-
tion used for qPCR (SEQ ID NOs: 27-34)

| | |
|---|---|
| pgcp_del1_F | CGATCAGTGGCTTCCTTCTC (SEQ ID NO: 27) |
| pgcp_del1_R | CGGACAATCCTGGCTTTTTA (SEQ ID NO: 28) |
| pgcp_del2_F | CGATGACCCCATAACATTCC (SEQ ID NO: 29) |
| pgcp_del2_R | AAGGTCTGGCTCCATCTGAA (SEQ ID NO: 30) |
| pgcp_con1_F | GTCAGCAACAGAGCCCTTTC (SEQ ID NO: 31) |
| pgcp_con1_R | CTCCCTCTGCTTGGAACTTG (SEQ ID NO: 32) |
| pgcp_con2_F | AGAACACTTTGGGGCACTTG (SEQ ID NO: 33) |
| pgcp_con2_R | GATTTCACACCCTGCTGACC (SEQ ID NO: 34) |

### Results:

Obsessive Compulsive Disorder (OCD) is a common (1-3% of the population) and debilitating neuropsychiatric disorder characterized by persistent intrusive thoughts and time-consuming repetitive behaviors [1]. Twin studies show OCD is very heritable (approximately 45-65% genetic influences for early onset OCD), but the underlying genetics is complex [2,3]. More than 80 candidate gene studies of OCD and a recent genome-wide association study (GWAS) yielded no significant, replicable associations [4]. The most strongly associated genes in the OCD GWAS implicate disrupted glutamatergic neurotransmission and signaling in disease pathogenesis [4].

Artificial mouse models have proven more effective for elucidating the neural pathways underlying OCD like behaviors. Mice lacking Sapap3, a postsynaptic scaffolding protein found at glutamatergic synapses, exhibited excessive grooming and increased anxiety, symptoms alleviated by treatment with selective serotonin reuptake inhibitors (SSRI), the same drug frequently used to treat OCD patients [5]. Optogenetic stimulation of the orbitofrontal cortex region affected by the Sapap3 mutation reversed defective neural activity and suppressed compulsive behavior [6]. Resequencing of exons of DLGAP3 (the human SAPAP3 gene) revealed excessive rare non-synonymous variants in human OCD and trichotillomania (TTM) individuals [7].

Canine OCD is a naturally occurring model for human OCD that is genetically more complex than induced animal models [8]. Phenotypically, canine and human OCD are remarkably similar. Canine compulsive disorder manifests as repetition of normal canine behaviors such as grooming (lick dermatitis), predatory behavior (tail chasing) and suckling (flank and blanket sucking). Just as in human patients, approximately 50% of dogs respond to the treatment with SSRIs or clomipramine [9]. Particular dog breeds (genetically isolated populations) have exceptionally high rates of OCD, including Doberman Pinschers (DP), bull terriers and German shepherds [10-12]. The high disease rates and rather limited genetic diversity of dog breeds suggests that OCD in these populations, while multi-genic, may be less complex than in humans, facilitating genetic mapping and functional testing of associated variants [13,14].

In this study, the MAGIC algorithm [15] was used to reanalyze data from a previous study and identify new OCD associated regions. These regions were enriched for genes involved in synapse formation and function, as are regions with patterns of reduced variation consistent with artificial selection. Top candidate regions were sequenced, totally 5.8 Mb, and it was found that four genes, all with synaptic function, were enriched for case-specific variants: neuronal-cadherin (CDH2), catenin alpha2 (CTNNA2), ataxin-1 (ATXN1), and plasma glutamate carboxypeptidase (PGCP). Furthermore, two intergenic mutations between the cadherin genes CDH2 and desmocollin 3 (DSC3) disrupted a non-coding regulatory element and alter gene expression in a human neuroblastoma cell line. The results implicate abnormal synapse formation and plasticity in OCD, and point to disrupted expression of neural cadherin genes as one possible cause.

GWAS and Homozygosity Mapping

The Affymetrix genotype intensity data was analyzed from the previous OCD GWAS with a new calling algorithm, MAGIC, that relaxes certain assumptions used in other callers, such as Hardy-Weinberg equilibrium in genotype clusters, to dramatically improve the accuracy of genotypes called from Affymetrix v2 Canine GeneChip data [15]. This yielded a 2.4 fold denser SNP map for association mapping (55,651 SNPs; 35,941 SNPs with MAF>0.05) but a slightly smaller sample size, with 87 cases and 63 controls passing MAGIC quality filters (compared to the original dataset of 14,700 SNPs with MAF>0.05 in 92 cases and 68 controls; FIGS. 1A and B). The increased density allowed us to identify 13 new candidate OCD-associated regions (p<0.0001) in addition to the original chromosome 7 locus in CDH2 (Table 7). It is estimated that this dataset explains 0.56±0.18 of phenotype variance [16].

All Gene Ontology gene sets were tested with 5-1000 genes (5206 sets) for enrichment in the new GWAS regions using INRICH, a permutation based software that rigorously controls for region size, SNP density, and gene size and gene number [17]. Overall, an excess of sets was observed with p<0.01 (25 sets, p=0.03, FIG. 1E). The top set “GO:0045295 Gamma catenin binding” was significant even after stringent correction for the number of gene sets tested (p=5.9×10⁻⁵, p_{corrected}=0.05) and included genes under each of three peaks of association spanning ˜3 Mb on chromosome 7: CDH2, DSC3 and DSG1 (FIGS. 1D and 1E; Table 13). The GWAS regions also included two of 13 genes in “GO:0048814 Regulation of dendrite morphogenesis” (p=0.002): the calcium binding synaptogenesis gene SDC2 and the postsynaptic density protein TNIK, a serine-threonine kinase involved in AMPA receptor trafficking and synaptic function [18,19].

TABLE 13
Enrichment analysis of GO catalog gene sets in associated regions.

| | Genes | Genes | | |
|---|---|---|---|---|
| TARGET | in set | in regions | P | Genes |
| GO:0045295 Gamma- | 12 | 3 | 6.9E−05 | CDH2; DSC3; DSG1 |
| catenin binding | | | | |
| GO:0004571 Mannosyl- | 9 | 2 | 0.0005 | EDEM3; MAN1A2 |
| oligosaccharide 1,2- | | | | |
| alpha-mannosidase | | | | |
| activity | | | | |
| GO:0030057 | 22 | 2 | 0.0015 | DSC3; |
| Desmosome | | | | DSG2, DSG3, DSG4, DSG1 |
| GO:0048814 Regulation | 13 | 2 | 0.0016 | TNIK; SDC2 |
| of dendrite | | | | |
| morphogenesis | | | | |
| GO:0036170 | 25 | 2 | 0.0020 | PLD1; MOB2 |
| Filamentous growth of a | | | | |
| population of unicellular | | | | |
| organisms in response to | | | | |
| starvation | | | | |
| GO:0005615 | 748 | 8 | 0.0021 | SCGB1D2, SCGB1D1; |
| Extracellular space | | | | RTN3; TTR; DKK1; |
| | | | | BPI, LBP; TNFSF10; CPQ; |
| | | | | CTSD |
| GO:0008344 Adult | 41 | 2 | 0.0037 | TRMT1L; ATXN1 |
| locomotory behavior | | | | |
| GO:0001523 Retinoid | 48 | 2 | 0.0042 | TTR; SDC2 |
| metabolic process | | | | |
| GO:0007603 | 50 | 2 | 0.0050 | TTR; SDC2 |
| Phototransduction, visible | | | | |
| light | | | | |
| GO:0043687 Post- | 181 | 3 | 0.0056 | EDEM3; MAN1A2; |
| translational protein | | | | GALNT14 |
| modification | | | | |
| GO:0051286 Cell tip | 5 | 2 | 0.0071 | SLC32A1; MOB2 |
| GO:0009267 Cellular | 48 | 2 | 0.0075 | PLD1; MOB2 |
| response to starvation | | | | |
| GO:0030447 | 56 | 2 | 0.0077 | PLD1; MOB2 |
| Filamentous growth | | | | |
| GO:0043202 Lysosomal | 64 | 2 | 0.0080 | SDC2; CTSD |
| lumen | | | | |
| GO:0016339 Calcium- | 25 | 2 | 0.0088 | CDH2; DSG1 |
| dependent cell-cell | | | | |
| adhesion | | | | |

The DP breed, like all dog breeds, was created through population bottlenecks and artificial selection for morphological and behavioral traits, potentially driving some OCD risk alleles to very high frequency and thus undetectable by GWAS. Consistent with this hypothesis, functional connections were found between associated genes and genes in the 13 largest autosomal regions of fixation in the DP breed (totally 25.7 Mb, Table 14). For example, the tyrosine kinase FER mediates cross talk between CDH2 and integrins [20], and depletion of presynaptic FER inhibits synaptic formation and transmission [21]. CTNNA2 interacts with CDH2 to regulate the stability of synaptic cell junctions [22]. While most fixed regions contained many genes, making it difficult to identify top candidates, several contained just one gene, including the neuronal protein LINGO2 and the synaptic-2 like glycoprotein gene TECRL.

TABLE 14
The thirteen longest regions of fixation in the DP.
Candidate genes identified in pathway analysis (FIG. 1) are underlined.

| chr | start | end | size | # genes | genes |
|---|---|---|---|---|---|
| 3 | 62948826 | 70302993 | 7.35 | 57 | ACOX3, METTL19, TRMT44, GPR78, CPZ, HMX1, ADRA2C, |
| | | | | | LRPAP1, DOK7, HGFAC, RGS12, HTT, GRK4, NOP14, |
| | | | | | MFSD10, ADD1, SH3BP2, TNIP2, FAM193A, RNF4, ZFYVE28, |
| | | | | | MXD4, HAUS3, POLN, NAT8L, C4orf48, WHSC2, NELFA, |
| | | | | | WHSC1, LETM1, FGFR3, TACC3, TMEM129, SLBP, |
| | | | | | FAM53A, UVSSA, MAEA, FAM184B, MED28, LAP3, CLRN2, QDPR, |
| | | | | | LDB2, TAPT1, PROM1, FGFBP1, CD38, BST1, FAM200B, |
| | | | | | FBXL5, CC2D2A, C1QTNF7, CPEB2, BOD1L, BOD1L1, NKX3- |
| | | | | | 2, RAB28 |
| 3 | 59513417 | 62897146 | 3.38 | 33 | MESDC2, KIAA1199, FAM108C1, ARNT2, FAH, ZFAND6, |
| | | | | | BCL2A1, MTHFS, KIAA1024, TMED3, RASGRF1, CTSH, |
| | | | | | MORF4L1, ADAMTS7, TBC1D2B, IDH3A, ACSBG1, DNAJA4, |
| | | | | | WDR61, CRABP1, PPP2R2C, MRFAP1, S100P, BLOC1S4, |
| | | | | | KIAA0232, TBC1D14, TADA2B, GRPEL1, SORCS2, PSAPL1, |
| | | | | | AFAP1, ABLIM2, SH3TC1 |
| 3 | 3548237 | 6087635 | 2.54 | 11 | EPB41L4A, NREP, STARD4, CAMK4, WDR36, TSLP, |
| | | | | | SLC25A46, TMEM232, MAN2A1, PJA2, FER |
| 24 | 3013164 | 4715848 | 1.70 | 10 | CST8, CST11, CSTL1, NAPB, GZF1, NXT1, CD93, THBD, |
| | | | | | SSTR4, FOXA2 |
| 31 | 7605474 | 9218454 | 1.61 | 1 | GBE1 |
| 17 | 45925444 | 47203813 | 1.28 | 2 | CTNNA2, LRRTM1 |
| 31 | 3075862 | 4279751 | 1.20 | 6 | CGGBP1, ZNF654, HTR1F, POU1F1, CHMP2B, VGLL3 |
| 12 | 36982027 | 38147867 | 1.17 | 2 | RIMS1, KCNQ5 |
| 14 | 3737103 | 4832311 | 1.10 | 17 | IBA57, GJC2, GUK1, MRPL55, ARF1, WNT3A, WNT9A, |
| | | | | | PRSS38, SNAP47, OR6F1, OR13G1, OR2AK2, OR2L13, |
| | | | | | OR2L3, OR2W3, TRIM58, OR11L1 |
| 2 | 62546927 | 63611219 | 1.06 | 13 | BBS2, OGFOD1, NUDT21, AMFR, GNAO1, CES5A, CES1, |
| | | | | | CES1P1, SLC6A2, LPCAT2, CAPNS2, MMP2, IRX6 |
| 13 | 57506475 | 58481113 | 0.97 | 1 | TECRL |
| 13 | 6295550 | 7242655 | 0.95 | 6 | GRHL2, NCALD, RRM2B, UBR5, ODF1, KLF10 |
| 11 | 49214235 | 50070536 | 0.86 | 1 | LINGO2 |

128 regions of unusually low variability were also identified in the DP breed compared to 24 other dog breeds (23.73 Mb, Table 15) [23]. When these regions of reduced variability (RRVs) were tested for gene set enrichment in the entire GO catalog, as described above, 10 GO terms were more enriched in DP RRVs than any other breed (FIG. 1F). Half of these have clear relevance to brain function, including regulation of neurotransmitters, neural projection, and dendrite morphogenesis. Enrichment is also seen for mannose binding related genes, echoing the strong enrichment in GWAS regions for alpha-mannosidase activity. Mannose structures are concentrated at excitatory synapses, including glutamate receptors [24,25].

TABLE 15
128 regions of reduced relative heterozygosity in the DP breed
Candidate genes identified in pathway analysis (FIG. 1) are underlined.

| CHR | START | END | GENES |
|---|---|---|---|
| 2 | 19440000 | 19590000 | none |
| 2 | 21780000 | 21990000 | MIR511-1, MIR511-2, SLC39A12, MRC1 |
| 2 | 80760000 | 80970000 | EIF4G3 |
| 3 | 66030000 | 66210000 | none |
| 3 | 68820000 | 68970000 | none |
| 4 | 6660000 | 6870000 | EDARADD, ERO1LB, GPR137B |
| 4 | 91290000 | 91440000 | ANKH |
| 5 | 12930000 | 13140000 | OR8B2, OR8B3, OR8B4, OR8B8 |
| 5 | 17160000 | 17370000 | PVRL1 |
| 5 | 42210000 | 42390000 | TRIM16L, ZNF286A, ZNF287, ZNF624 |
| 5 | 45690000 | 45930000 | KCNJ12, MAP2K3 |
| 5 | 81840000 | 81990000 | none |
| 6 | 12510000 | 12660000 | AZGP1, COPS6, ZKSCAN1, ZNF3, ZSCAN21 |
| 6 | 14100000 | 14250000 | BHLHA15, LMTK2, TECPR1 |
| 6 | 14610000 | 14760000 | C7orf70, CYTH3, FAM220A, RAC1 |
| 6 | 39630000 | 39900000 | SEPT12, ANKS3, FAM100A, GLYR1, MGRN1, ROGDI, ZNF500 |
| 7 | 55320000 | 55530000 | none |
| 7 | 69300000 | 69450000 | ABHD3, MIB1, SNRPD1 |
| 8 | 9540000 | 9690000 | none |
| 8 | 11910000 | 12120000 | PRKD1 |
| 8 | 19680000 | 19860000 | none |
| 8 | 21060000 | 21240000 | none |
| 8 | 25110000 | 25320000 | none |
| 8 | 33330000 | 33480000 | CDKN3, CGRRF1, CNIH, GMFB |
| 8 | 60810000 | 61020000 | none |
| 8 | 67320000 | 67500000 | C14orf49, GLRX5, SNHG10 |
| 8 | 70860000 | 71010000 | CCDC85C, CCNK, SETD3 |
| 8 | 71310000 | 71490000 | EML1, EVL, MIR342 |
| 9 | 7890000 | 8040000 | LLGL2, RECQL5, SAP30BP, TSEN54 |
| 9 | 15450000 | 15720000 | BPTF, CEP95, SMURF2 |
| 9 | 16110000 | 16320000 | PITPNC1, PSMD12 |
| 9 | 18660000 | 18840000 | ABCA10, ABCA5, ABCA6, ABCA9 |
| 9 | 51030000 | 51180000 | ATP2A3, CACNA1B |
| 10 | 11580000 | 11730000 | IRAK3, LLPH, TMBIM4 |
| 10 | 31920000 | 32070000 | HMGXB4, ISX |
| 11 | 6270000 | 6420000 | AGXT2L2, COL23A1, HNRNPAB, N4BP3, NHP2, RMND5B |
| 11 | 36690000 | 36870000 | MPDZ |
| 11 | 38310000 | 38490000 | FREM1 |
| 11 | 41850000 | 42030000 | FAM154A, HAUS6, PLIN2, RRAGA, SCARNA8 |
| 11 | 52230000 | 52380000 | none |
| 12 | 14340000 | 14490000 | CNPY3, PTCRA, RPL7L1 |
| 12 | 19500000 | 19650000 | none |
| 12 | 35490000 | 35640000 | COL19A1 |
| 12 | 36660000 | 36930000 | LINC00472, MIR30A, MIR30C2, OGFRL1 |
| 12 | 38220000 | 38370000 | KCNQ5, KHDC1, KHDC1L |
| 12 | 63780000 | 64050000 | none |
| 12 | 65940000 | 66120000 | none |
| 13 | 5880000 | 6030000 | ZNF706 |
| 13 | 45210000 | 45360000 | GABRA2, GABRG1 |
| 13 | 53490000 | 53700000 | none |
| 13 | 61080000 | 61230000 | GNRHR, UBA6 |
| 14 | 48390000 | 48540000 | BBS9 |
| 15 | 3390000 | 3600000 | C1orf50, ERMAP, LOC100129924, SLC2A1 |
| 15 | 6870000 | 7110000 | MYCBP, RRAGC |
| 15 | 16290000 | 16440000 | CMPK1, STIL, FOXE3 |
| 15 | 19950000 | 20100000 | EBNA1BP2, WDR65 |
| 15 | 24450000 | 24600000 | SYT1 |
| 15 | 28170000 | 28350000 | none |
| 15 | 33930000 | 34140000 | none |
| 15 | 40650000 | 40860000 | none |
| 15 | 53460000 | 53730000 | ARFIP1, TIGD4, TMEM154 |
| 15 | 63900000 | 64050000 | TRIM61 |
| 15 | 65520000 | 65790000 | SPOCK3 |
| 16 | 56040000 | 56190000 | none |
| 17 | 18060000 | 18210000 | LAPTM4A, SDC1 |
| 17 | 46440000 | 46650000 | CTNNA2 |
| 17 | 65040000 | 65310000 | LOR, MAGI3 |
| 18 | 7920000 | 8130000 | HPVC1 |
| 18 | 15390000 | 15630000 | LAMB1, LAMB4, NRCAM |
| 18 | 51570000 | 51780000 | MYEOV |
| 19 | 13680000 | 13830000 | none |
| 19 | 21390000 | 21540000 | ANXA5 |
| 19 | 21630000 | 21780000 | QRFPR |
| 19 | 23460000 | 23610000 | FAM123C |
| 19 | 37080000 | 37230000 | DPP10 |
| 20 | 8580000 | 8790000 | C3orf25, CAND2, H1FOO, IFT122, PLXND1, RPL32, RHO |
| 20 | 16050000 | 16260000 | ITPR1, SETMAR, SUMF1 |
| 20 | 17580000 | 17790000 | CNTN4 |
| 20 | 20160000 | 20340000 | none |
| 20 | 23970000 | 24120000 | FOXP1 |
| 20 | 32190000 | 32370000 | PTPRG |
| 20 | 33480000 | 33660000 | FHIT |
| 20 | 61020000 | 61200000 | BSG, C2CD4D, CDC34, FGF22, FSTL3, HCN2, MADCAM1, ODF3L2, POLRMT, |
| | | | PRSS57, RNF126, SHC2, THEG, TPGS1 |
| 21 | 51570000 | 51780000 | KIF18A, METTL15 |
| 22 | 20250000 | 20400000 | none |
| 22 | 29160000 | 29340000 | none |
| 22 | 32220000 | 32400000 | COMMD6, LMO7, TBC1D4, UCHL3 |
| 22 | 47310000 | 47520000 | GPC6 |
| 23 | 8850000 | 9000000 | ARPP21 |
| 23 | 18480000 | 18630000 | none |
| 23 | 24480000 | 24750000 | ZNF385D |
| 24 | 3960000 | 4140000 | LINC00261, FOXA2 |
| 24 | 10560000 | 10740000 | MACROD2 |
| 24 | 44490000 | 44670000 | AURKA, C20orf43, CASS4, CSTF1, FAM210B, GCNT7 |
| 25 | 28860000 | 29100000 | CTSB, DEFB131, DEFB134, DEFB135, DEFB136, FDFT1, LOC100129216, NEIL2 |
| 25 | 39720000 | 39900000 | ATP6V1B2, SLC18A1, LZTS1 |
| 25 | 49350000 | 49500000 | AGAP1 |
| 25 | 53250000 | 53520000 | GPC1, MIR149, MYEOV2, OTOS, PP14571 |
| 26 | 3630000 | 3780000 | GALNT9, LOC100130238 |
| 26 | 4260000 | 4410000 | GPR133 |
| 26 | 24270000 | 24570000 | MN1, PITPNB, TTC28, TTC28-AS1 |
| 26 | 33300000 | 33510000 | CCDC74A, CCDC74B, KLHL22, MED15, MZT2A, MZT2B, SCARF2, SMPD4, |
| | | | TUBA3C |
| 26 | 33990000 | 34200000 | MAPK1, PPIL2, YPEL1 |
| 26 | 38880000 | 39030000 | PRKG1 |
| 26 | 40800000 | 41010000 | ATAD1, KLLN, PAPSS2, PTEN |
| 27 | 3780000 | 3960000 | GPR84, GTSF1, ITGA5, NCKAP1L, PDE1B, ZNF385A |
| 27 | 21480000 | 21660000 | ERGIC2, OVCH1, TMTC1 |
| 27 | 37260000 | 37440000 | ETV6 |
| 28 | 35730000 | 36000000 | none |
| 28 | 36330000 | 36480000 | CHST15, CPXM2 |
| 28 | 39990000 | 40140000 | none |
| 29 | 19080000 | 19230000 | C8orf46, MYBL1, VCPIP1 |
| 29 | 20910000 | 21060000 | C8orf34 |
| 29 | 32580000 | 32730000 | none |
| 29 | 33600000 | 33840000 | RALYL |
| 30 | 14850000 | 15060000 | PLDN, SLC30A4, SQRDL |
| 30 | 22650000 | 22800000 | UNC13C |
| 30 | 37710000 | 37890000 | CT62, LRRC49, THSD4 |
| 32 | 14340000 | 14490000 | ABCG2, PKD2 |
| 33 | 12600000 | 12780000 | none |
| 33 | 33060000 | 33210000 | DLG1 |
| 34 | 10110000 | 10260000 | LOC255167, NSUN2, SRD5A1, UBE2QL1 |
| 34 | 16830000 | 17010000 | CCDC39, TTC14 |
| 34 | 32670000 | 32820000 | none |
| 36 | 7200000 | 7350000 | CCDC148, PKP4 |
| 36 | 21270000 | 21510000 | CIR1, GPR155, OLA1, SCRN3 |
| 38 | 19470000 | 19740000 | none |
| 35 | 3240000 | 3510000 | none |

A sequencing array was designed (Table 8 and Table 9) that targeted nine of the top GWAS regions, including the CDH2 locus (3.9 Mb; FIG. 5) as well as genes and conserved elements within the five largest DP fixed regions (Table 14). Focus was on fixed regions (totaling 1.8 Mb) that were also fixed in two other OCD affected breeds, German shepherds (LUPA reference panel [26]) and bull terriers (20 dogs) (FIG. 1B,C). Eight cases and eight matched controls were sequenced from breeds at high risk for OCD, including eight DP, four German shepherds, two Shetland sheepdogs and two Jack Russell terriers (FIG. 2A). DPs were selected based on their genotype for the CDH2 risk haplotype [14] (two homozygous cases, two heterozygous cases, and four controls without the risk haplotype). 92% of the target regions were captured at >20× coverage, with 76× mean read depth coverage per sample (Table 16). In total, 24,930 high-quality SNPs were detected, 7,645 short INDELs, and 173 deletions, with high concordance to the SNP array data (median 99.5% for ˜390 SNPs tested per sample; Table 17). In Table 17 variants were detected through GATK pipeline and all the samples were genotyped using IIlumina CanineHD BeadChip to validate the SNP call from sequencing data.

TABLE 16
Sequencing statistics

| | | | | PCT of Target | PCT of Target |
|---|---|---|---|---|---|
| | Unique Bases | On Target | Mean Target | Bases with | Bases with |
| Sample ID | Aligned | Bases | Coverage | coverage ≧2x | covergae ≧20x |
| DP CASE 15237 | 1,139,683,488 | 344,896,305 | 69.7 | 99.3 | 90.8 |
| DP CASE 15255 | 1,086,228,716 | 412,287,216 | 83.2 | 99.4 | 94.2 |
| DP CASE 15259 | 1,110,107,291 | 429,179,317 | 86.5 | 99.5 | 94.1 |
| DP CASE 15260 | 951,139,666 | 282,485,917 | 57.1 | 99.2 | 85.8 |
| DP CONTROL 15227 | 1,154,846,994 | 443,969,022 | 89.6 | 99.3 | 94.4 |
| DP CONTROL 15253 | 1,050,532,650 | 387,532,268 | 78.2 | 99.3 | 93.2 |
| DP CONTROL 15254 | 1,143,518,339 | 436,687,467 | 88.0 | 99.5 | 94.1 |
| DP CONTROL 15262 | 945,386,032 | 340,943,650 | 68.9 | 99.0 | 88.6 |
| GS CASE 5913 | 1,133,632,267 | 312,071,917 | 63.0 | 99.4 | 91.3 |
| GS CASE 5990 | 1,162,734,835 | 330,699,135 | 66.8 | 99.2 | 90.8 |
| GS CONTROL 2722 | 910,366,662 | 385,183,461 | 77.7 | 99.5 | 93.5 |
| GS CONTROL 5989 | 948,552,788 | 406,781,333 | 82.0 | 99.5 | 94.5 |
| JR CASE 3094 | 1,267,941,716 | 368,157,308 | 74.3 | 99.4 | 92.1 |
| JR CONTROL 205 | 1,062,769,474 | 305,843,637 | 61.7 | 99.3 | 89.4 |
| SS CASE 5991 | 974,962,641 | 419,839,514 | 84.7 | 99.5 | 95.0 |
| SS CONTROL 1737 | 945,271,757 | 405,413,251 | 81.7 | 99.5 | 94.6 |

TABLE 17
Variants detected in each individual.

| | | | | | | SNP Chip |
|---|---|---|---|---|---|---|
| Sample ID | SNPs | Insertions | Deletions | Variants | Ti/Tv | Concordance (%) |
| DP CASE 15237 | 10757 | 1815 | 2533 | 15105 | 2.58 | 99.0 |
| DP CASE 15255 | 12211 | 1993 | 2869 | 17073 | 2.66 | 99.7 |
| DP CASE 15259 | 10926 | 1839 | 2640 | 15405 | 2.66 | 99.7 |
| DP CASE 15260 | 9063 | 1619 | 2254 | 12936 | 2.57 | 99.0 |
| DP CONTROL 15227 | 11333 | 1902 | 2748 | 15983 | 2.68 | 99.7 |
| DP CONTROL 15253 | 11399 | 1903 | 2753 | 16055 | 2.61 | 99.2 |
| DP CONTROL 15254 | 9458 | 1713 | 2524 | 13695 | 2.57 | 99.5 |
| DP CONTROL 15262 | 10803 | 1830 | 2672 | 15305 | 2.6 | 99.5 |
| GS CASE 5913 | 12399 | 2045 | 2740 | 17184 | 2.56 | 99.7 |
| GS CASE 5990 | 11330 | 1861 | 2613 | 15804 | 2.59 | 99.5 |
| GS CONTROL 2722 | 11100 | 1895 | 2675 | 15670 | 2.57 | 99.7 |
| GS CONTROL 5989 | 10464 | 1825 | 2648 | 14937 | 2.57 | 99.5 |
| JR CASE 3094 | 12617 | 2048 | 2744 | 17409 | 2.62 | 99.2 |
| JR CONTROL 205 | 11769 | 1929 | 2703 | 16401 | 2.63 | 97.2 |
| SS CASE 5991 | 10129 | 1787 | 2599 | 14515 | 2.55 | 99.5 |
| SS CONTROL 1737 | 11617 | 2061 | 2872 | 16550 | 2.55 | 93.7 |

Case-Only Variant Discovery from Sequence Data

With the small sample size (8 cases and 8 controls from four different breeds), the study was not expected to have sufficient power to detect statistically significant allelic associations with OCD. Instead, focused was on variants seen only in OCD cases (“case-only variants”) as the strongest causal candidates. Of 32,575 variants, 2,291 variants are case-only (2,002 SNPs and 289 INDELs; 80-966 per dog), while 3,116 variants are specific to control dogs (“control-only variants”; 2,698 SNPs and 418 INDELs; 156-1,476 per dog) (Table 18 and Table 19). While there is no significant difference between the total number of case- and control-only variants (Wilcoxon test p=0.63; FIG. 2B), case dogs had a significantly greater number in evolutionarily constrained elements (median 15 vs. 4, Wilcoxon test p=0.02; see methods; FIG. 2B and Table 19). Excluding coding variants increased the difference further (median 15 vs. 3, Wilcoxon test p=0.01), suggesting that the excess of case-only functional variants may be due largely to noncoding variation.

TABLE 18
Sequence variants identified by targeted resequencing
of 5.8 Mb in eight cases and eight controls

| | All | Variants | Variants | Case- | Control- |
|---|---|---|---|---|---|
| | 16 | in | in | only | only |
| Annotations | dogs | cases | controls | variants | variants |
| All Variants | 32,575 | 29,425 | 30,253 | 2,291 | 3,116 |
| Missense | 71 | 64 | 61 | 6 | 7 |
| mutations | | | | | |
| Nonsense | 0 | 0 | 0 | 0 | 0 |
| mutations | | | | | |
| Frame-shift | 2 | 3 | 3 | 0 | 0 |
| mutations | | | | | |
| Silent coding | 108 | 97 | 89 | 18 | 11 |
| variants | | | | | |
| UTR variants | 22 | 16 | 22 | 0 | 6 |
| Essential Splice | 1 | 1 | 1 | 0 | 0 |
| Site | | | | | |
| Conserved sites* | 1,024 | 930 | 908 | 119 | 91 |

*Conserved sites determined by 29 mammals sequence data set [27]

In Table 19 all unique variants (AUV) show the counts of variants that were present in any case but not any control for CASE sub-table and vice versa for CONTROL sub-table; conserved unique variants (CUV) show variants from all unique variants (AUV) column that are within conserved elements. P-values were calculated by paired one-sided Wilcoxon signed rank test; P-value* excluded the lower-quality Shetland Sheepdog pairs. Sample names include breed as DP (Doberman pinscher), SS (Shetland Sheepdog), JR (Jack Russell terrier) and GS (German shepherd).

TABLE 19
Case-/Control- Only Variants in Each Dog

| | | |
|---|---|---|
| All Unique | Conserved Unique | Conserved Unique |
| Variants | Variants | noncoding |

CASE

| | | | |
|---|---|---|---|
| DP 15237 | 233 | 12 | 12 |
| DP 15259 | 80 | 6 | 4 |
| DP 15255 | 426 | 19 | 19 |
| DP 15260 | 290 | 15 | 15 |
| SS 5991 | 437 | 27 | 14 |
| JR 3094 | 966 | 47 | 42 |
| GS 5913 | 383 | 14 | 13 |
| GS 5990 | 422 | 26 | 23 |

CONTROL

| | | | |
|---|---|---|---|
| DP 15227 | 156 | 3 | 3 |
| DP 15253 | 335 | 6 | 5 |
| DP 15254 | 207 | 4 | 1 |
| DP 15262 | 275 | 6 | 6 |
| SS 1737 | 1476 | 41 | 39 |
| JR 205 | 1204 | 42 | 37 |
| GS 2722 | 130 | 4 | 1 |
| GS 5989 | 204 | 1 | 1 |
| P-value | 0.63 | 0.075 | 0.09 |
| P-value* | 0.41 | 0.018 | 0.011 |

Genotyping Case-Only Variants in Independent Samples

Case-specific variants were selected that were within evolutionarily constrained elements determined by 29 mammals sequence dataset [27]. Then, a subset of the variants were selected meeting one of the following criteria: (i) case-only variants within DP breed; (ii) case-only variants within CDH2, PGCP, CTNNA2 and ATXN1 that were identified by gene-based analysis; (iii) case-only variants across at least two breeds; (iv) potential functional variants annotated as nonsense, splicing or missense (predicted to be “probably” or “possibly damaging” by Polyphen-2 [66]) and case-only variants in at least one breed; (v) variants within CDH2 risk haplotype; and (vi) top associated variants from GWA-analysis. Of 140 variants that met one of the criteria, 127 variants passed Sequenom design standards, and were genotyped using the Sequenom iPlex system. After genotype call quality control, 114 variants (SNPs) were remained in the dataset. The complete list of 140 variants (SNPs) are shown in Table 3.

The 114 case-only, evolutionarily constrained variants were genotyped in an independent set of dogs from breeds with high rates of OCD (“OCD-risk breeds”; 69 dogs) and breeds with normal rates of OCD and other psychiatric disorders (“control breeds”; 19 dogs). Except for 14 cases from OCD-risk breeds, there was no individual OCD phenotype information for these dogs (FIG. 2A). The case-only variants identified in the sequence data were significantly more common in OCD-risk breeds, with median frequency (FOCD) of 0.17, than in control breeds, where the median frequency (Fcontrol) is 0.05 (Wilcoxon test p=0.045; Table 20). In Table 20 the SNP column indicates [chromosome.base position], A1 and A2 columns indicate reference and alternative alleles, respectively and F_A and F_U indicate the frequency of alternative alleles in OCD-enriched and control breeds, respectively. The median frequency increases to 0.20 when only phenotyped cases are considered (Wilcoxon test p=0.015, comparison with Fcontrol). An inverse correlation was observed between the frequency difference between OCD-risk and control breeds and the frequency across all genotyped dogs (Pearson's R=−0.63, p=8.4×10-14; FIG. 2C). Thus, the variants most enriched in OCD-risk breeds were otherwise rare, potentially due either to positive selective pressure in OCD-risk breeds or negative selection in the control breeds. While this suggests an association with OCD, other traits may also systematically differ between the two breed groups.

TABLE 20
Genotypes of 114 candidate SNPs on 88 dogs.

| SNP | A1 | F_A | F_U | A2 |
|---|---|---|---|---|
| chr17.46715746 | A | 0.007246 | 0 | T |
| chr17 46791415 | T | 0.007246 | 0 | C |
| chr18.48938780 | T | 0.007246 | 0 | C |
| chr29.44249614 | C | 0.007246 | 0 | T |
| chr35.18484947 | G | 0 | 0.02632 | A |
| chr18.57136368 | A | 0.01449 | 0 | T |
| chr29.44205914 | G | 0.01449 | 0 | A |
| chr29.44205937 | G | 0.01449 | 0 | A |
| chr29.44205943 | A | 0.01449 | 0 | G |
| chr29.44306347 | A | 0.01449 | 0 | G |
| chr35.18850625 | G | 0.01449 | 0 | T |
| chr35.18861596 | A | 0.01449 | 0 | G |
| chr7.63832008 | G | 0.02174 | 0 | A |
| chr17.46897529 | G | 0.02174 | 0 | A |
| chr29.44300177 | A | 0.02174 | 0 | G |
| chr35.18860763 | T | 0.02174 | 0 | C |
| chr7.63891778 | T | 0.02174 | 0.07895 | C |
| chr18.56889625 | T | 0.02899 | 0.02632 | C |
| chr29.44422867 | G | 0.04348 | 0 | T |
| chr7.61693835 | C | 0.03623 | 0 | T |
| chr7.63852056 | T | 0.05072 | 0 | C |
| chr3.65472187 | A | 0.05797 | 0 | G |
| chr7.61669045 | A | 0.05797 | 0.02632 | T |
| chr7.61693952 | C | 0.05797 | 0.02632 | G |
| chr35.18857719 | A | 0.05797 | 0.02632 | T |
| chr19.13696914 | C | 0.07246 | 0 | T |
| chr35.18679978 | C | 0.07246 | 0 | G |
| chr3.64690526 | A | 0.07246 | 0.02632 | G |
| chr7.63966490 | A | 0.07246 | 0 | C |
| chr29.44437957 | C | 0.05797 | 0.1053 | T |
| chr7.61728453 | C | 0.05072 | 0.1053 | T |
| chr7.63802530 | T | 0.07971 | 0.02632 | C |
| chr29.44306628 | G | 0.0942 | 0 | A |
| chr7.63796858 | T | 0.07971 | 0.02632 | G |
| chr7.63796857 | A | 0.07971 | 0.05263 | C |
| chr7.63852467 | C | 0.1014 | 0 | T |
| chr3.65188233 | G | 0.1159 | 0 | A |
| chr29.44393030 | G | 0.1014 | 0 | A |
| chr29.44397446 | G | 0.1232 | 0 | T |
| chr7.63806661 | G | 0.1087 | 0.02632 | A |
| chr7.63943045 | A | 0.1304 | 0 | G |
| chr7.63950125 | A | 0.1304 | 0 | G |
| chr35.18464093 | A | 0.07971 | 0.2105 | T |
| chr29.44447800 | T | 0.08696 | 0.1944 | C |
| chr7.63779775 | G | 0.0942 | 0.1316 | C |
| chr7.63943118 | A | 0.1377 | 0 | T |
| chr3.5470809 | C | 0.05797 | 0.3421 | T |
| chr17.27881676 | T | 0.1304 | 0.1579 | C |
| chr29.44180170 | A | 0.1765 | 0 | C |
| chr3.3896965 | A | 0.1812 | 0 | G |
| chr18.56754830 | A | 0.1594 | 0.05263 | G |
| chr18.56768794 | T | 0.1739 | 0 | C |
| chr3.3894924 | A | 0.1884 | 0 | G |
| chr7.63866863 | T | 0.1812 | 0.02632 | G |
| chr17.46781512 | A | 0.1884 | 0.02632 | G |
| chr3.64769048 | A | 0.1014 | 0.3947 | C |
| chr17.46791238 | C | 0.1449 | 0.2368 | T |
| chr18.57174849 | A | 0.1812 | 0.1316 | G |
| chr29.44338785 | A | 0.1667 | 0.1842 | G |
| chr17.46607594 | C | 0.2174 | 0.02632 | T |
| chr18.56737581 | T | 0.1522 | 0.2368 | C |
| chr29.44353724 | T | 0.1739 | 0.1842 | C |
| chr17.46478790 | C | 0.2029 | 0.1053 | T |
| chr3.61823869 | C | 0.1232 | 0.4211 | T |
| chr17.46781268 | C | 0.2391 | 0.02632 | T |
| chr7.63870467 | G | 0.2391 | 0.02632 | A |
| chr7.63870482 | C | 0.2391 | 0.02632 | A |
| chr7.63870496 | G | 0.2391 | 0.02632 | A |
| chr7.63870599 | G | 0.2391 | 0.02632 | A |
| chr3.5754697 | A | 0.1765 | 0.3333 | G |
| chr3.5471514 | G | 0.1739 | 0.3421 | A |
| chr3.5754700 | A | 0.1739 | 0.3684 | G |
| chr3.6063747 | G | 0.2029 | 0.3056 | A |
| chr18.56898066 | G | 0.1522 | 0.5 | A |
| chr29.44392979 | T | 0.1957 | 0.2632 | C |
| chr18.48821291 | T | 0.2391 | 0.2105 | C |
| chr7.63867146 | T | 0.1957 | 0.3947 | A |
| chr34.39420664 | T | 0.2754 | 0.1579 | G |
| chr29.44152594 | A | 0.2536 | 0.2632 | C |
| chr17.46791139 | C | 0.1812 | 0.5789 | G |
| chr18.48823350 | G | 0.2391 | 0.3684 | A |
| chr7.63866105 | A | 0.2319 | 0.4211 | C |
| chr17.46617340 | A | 0.2899 | 0.2778 | G |
| chr3.65472276 | A | 0.2391 | 0.4737 | G |
| chr17.46722666 | G | 0.3043 | 0.2632 | C |
| chr7.63845290 | T | 0.3768 | 0 | C |
| chr7.63857947 | T | 0.3551 | 0.1053 | C |
| chr7.63868442 | C | 0.3696 | 0.1053 | T |
| chr7.63912017 | A | 0.3986 | 0 | G |
| chr29.44336600 | T | 0.3551 | 0.1579 | G |
| chr7.63814306 | A | 0.3986 | 0.02778 | G |
| chr7.63814172 | T | 0.3986 | 0.05263 | C |
| chr7.63860234 | A | 0.3696 | 0.1579 | G |
| chr29.44513249 | G | 0.2754 | 0.5789 | A |
| chr29.44392126 | G | 0.2424 | 0.6316 | A |
| chr7.63845160 | T | 0.4348 | 0 | A |
| chr7.63870150 | A | 0.3116 | 0.5 | G |
| chr7.63814541 | C | 0.413 | 0.1579 | A |
| chr3.5559765 | G | 0.3623 | 0.4211 | A |
| chr7.63867618 | G | 0.3478 | 0.5 | C |
| chr7.63867879 | T | 0.3551 | 0.5 | A |
| chr34.38986422 | A | 0.3551 | 0.5 | G |
| chr29.44244625 | G | 0.4265 | 0.2105 | A |
| chr7.63870805 | T | 0.3623 | 0.5 | A |
| chr7.61865715 | A | 0.4348 | 0.2222 | G |
| chr7.63868034 | T | 0.3913 | 0.4737 | C |
| chr7.63867472 | C | 0.3913 | 0.5 | T |
| chr7.63868258 | T | 0.3913 | 0.5 | C |
| chr29.44489802 | A | 0.413 | 0.4737 | C |
| chr3.5681788 | T | 0.4275 | 0.5526 | G |
| chr7.63921141 | C | 0.5652 | 0.05263 | T |
| chr34.38956076 | G | 0.4203 | 0.6053 | A |
| chr7.63872172 | G | 0.4275 | 0.6316 | C |
| chr34.39405162 | A | 0.5662 | 0.2368 | G |

Gene-Based Analysis

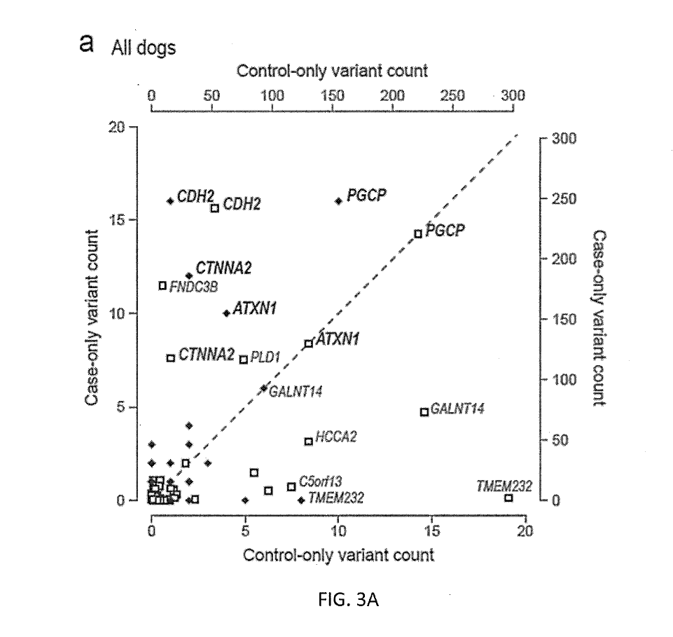

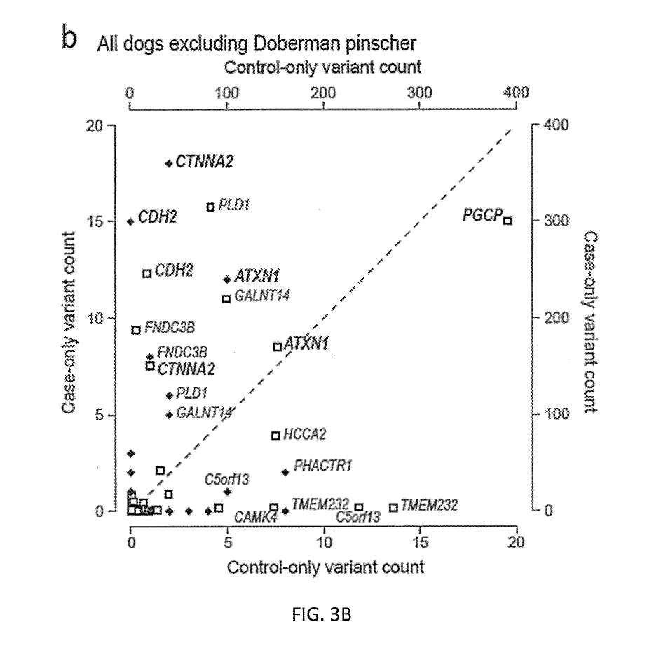

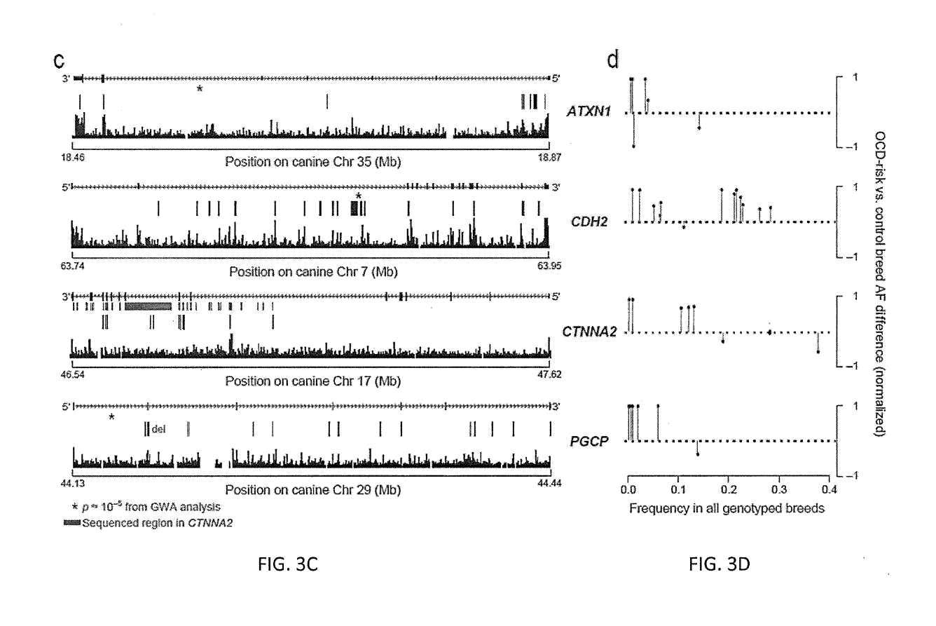

Genes enriched with case-only variants were identified using a gene-based analysis method that accounts for multiple independent variants within a gene and greatly increases power for identifying disease-associated genes [28]. Four genes had an excess of case-only variation in evolutionarily constrained elements, even after correcting for gene size: ATXN1, CDH2, CTNNA2, and PGCP (10, 16, 12, and 16 case-only variants respectively; FIG. 3A and Table 21). In Table 21 ALL signifies all dogs; DP represents Doberman; OTHERS includes all dogs excluding DP; Ratio is the (# case-only variants)/(# control only variants+1) within a gene and its 5 kb flanking. Because the sequenced DPs were selected based on their haplotype at CDH2, it was confirmed that the case-only enrichment at CDH2 persists even when DPs were excluded (FIG. 3B). RNA-Seq data shows all four genes were expressed in the dog brain. Three of the four candidate genes, CDH2, PGCP and ATXN1, were associated with OCD in the DP GWAS study (chr7:63867472, p=2.1×10-5; chr29:44152594, p=1.5×10-5; chr35:18565131, p=1.6×10-5 respectively), while the fourth, CTNNA2, fell in a large region of fixation (900 kb) in the DP breed (FIG. 3C). In the genotyping dataset, the case-only variants in these four genes were more common in OCD-risk breeds (FOCD=0.08 vs. Fcontrol=0.026, Wilcoxon test p=2.95×10-4; FIG. 3D), particularly in CDH2 (FOCD=0.23 vs. Fcontrol=0.027, p=0.001) and in PGCP (FOCD=0.014 vs. Fcontrol=0.0, p=0.047). A similar, though weaker pattern in ATXN1 (FOCD=0.022 vs. Fcontrol=0.0, p=0.3) and CTNNA2 (FOCD=0.185 vs. Fcontrol=0.026, p=0.13) is observed. In CTNNA2, the difference was clearer (p=0.054) if only variants with frequency <0.20 were considered.

TABLE 21
Ratio of Case/Control-Only Variants within Conserved Elements of Genes.

| | ALL | | | DP | | | OTHERS | | |
|---|---|---|---|---|---|---|---|---|---|
| Gene | Case | Control | Ratio | Case | Control | Ratio | Case | Control | Ratio |
| CDH2 | 16 | 1 | 8.0 | 16 | 6 | 2.3 | 15 | 0 | 15.0 |
| CTNNA2 | 12 | 2 | 4.0 | 0 | 2 | 0.0 | 18 | 2 | 6.0 |
| ZFYVE28 | 3 | 0 | 3.0 | NA | NA | NA | 3 | 0 | 3.0 |
| ATXN1 | 10 | 4 | 2.0 | 1 | 10 | 0.1 | 12 | 5 | 2.0 |
| TMEM212 | 2 | 0 | 2.0 | NA | NA | NA | 1 | 0 | 1.0 |
| CHRM1 | 2 | 0 | 2.0 | NA | NA | NA | 1 | 0 | 1.0 |
| KIAA1530 | 2 | 0 | 2.0 | NA | NA | NA | 1 | 0 | 1.0 |
| NOP14 | 2 | 0 | 2.0 | NA | NA | NA | 1 | 0 | 1.0 |
| SLC22A8 | 2 | 0 | 2.0 | 2 | 0 | 2.0 | 2 | 0 | 2.0 |
| PGCP | 16 | 10 | 1.5 | 0 | 2 | 0.0 | 23 | 16 | 1.4 |
| FNDC3B | 4 | 2 | 1.3 | 0 | 4 | 0.0 | 8 | 1 | 4.0 |
| CAPN14 | 2 | 1 | 1.0 | NA | NA | NA | 2 | 0 | 2.0 |
| PLD1 | 3 | 2 | 1.0 | 0 | 1 | 0.0 | 6 | 2 | 2.0 |
| HAUS3 | 1 | 0 | 1.0 | NA | NA | NA | 1 | 0 | 1.0 |
| MXD4 | 1 | 0 | 1.0 | NA | NA | NA | 1 | 0 | 1.0 |
| SLC22A6 | 1 | 0 | 1.0 | 1 | 0 | 1.0 | 1 | 0 | 1.0 |
| SORCS2 | 1 | 0 | 1.0 | NA | NA | NA | 1 | 0 | 1.0 |
| TADA2B | 1 | 0 | 1.0 | NA | NA | NA | 1 | 0 | 1.0 |
| TBC1D14 | 1 | 0 | 1.0 | NA | NA | NA | 1 | 0 | 1.0 |
| TNIP2 | 1 | 0 | 1.0 | NA | NA | NA | 1 | 0 | 1.0 |
| WDR74 | 1 | 0 | 1.0 | NA | NA | NA | NA | NA | NA |
| GALNT14 | 6 | 6 | 0.9 | 0 | 19 | 0.0 | 5 | 2 | 1.7 |
| PHACTR1 | 2 | 3 | 0.5 | 0 | 2 | 0.0 | 2 | 8 | 0.2 |
| KIAA0232 | 1 | 1 | 0.5 | NA | NA | NA | 1 | 0 | 1.0 |
| LRRTM1 | 1 | 1 | 0.5 | NA | NA | NA | 0 | 1 | 0.0 |
| KRTAP5-8 | 1 | 2 | 0.3 | 1 | 2 | 0.3 | 3 | 0 | 3.0 |
| C5orf13 | 1 | 2 | 0.3 | 3 | 0 | 3.0 | 1 | 5 | 0.2 |
| CAMK4 | 1 | 2 | 0.3 | 1 | 0 | 1.0 | 0 | 4 | 0.0 |
| AHNAK | 0 | 1 | 0.0 | 1 | 0 | 1.0 | 0 | 1 | 0.0 |
| DUSP8 | 0 | 1 | 0.0 | 2 | 1 | 1.0 | NA | NA | NA |
| MOB2 | 0 | 1 | 0.0 | NA | NA | NA | 0 | 2 | 0.0 |
| TSPYL5 | 0 | 1 | 0.0 | NA | NA | NA | 0 | 1 | 0.0 |
| FAM193A | 0 | 1 | 0.0 | NA | NA | NA | 0 | 1 | 0.0 |
| MAN2A1 | 0 | 5 | 0.0 | NA | NA | NA | 0 | 3 | 0.0 |
| PJA2 | 0 | 2 | 0.0 | 1 | 0 | 1.0 | 0 | 2 | 0.0 |
| STX5 | 0 | 1 | 0.0 | 1 | 3 | 0.3 | 1 | 0 | 1.0 |
| TMEM232 | 0 | 8 | 0.0 | NA | NA | NA | 0 | 8 | 0.0 |
| WDR36 | 0 | 1 | 0.0 | NA | NA | NA | 0 | 1 | 0.0 |
| HCCA2 | NA | NA | NA | 1 | 0 | 1.0 | 0 | 1 | 0.0 |
| TNFSF10 | NA | NA | NA | 0 | 1 | 0.0 | NA | NA | NA |
| EPB41L4A | NA | NA | NA | 1 | 0 | 1.0 | 0 | 1 | 0.0 |
| FER | NA | NA | NA | 3 | 0 | 3.0 | 0 | 2 | 0.0 |
| MFSD10 | NA | NA | NA | NA | NA | NA | 1 | 0 | 1.0 |

The coordinates for the genes with case/control-only variants and their potential regulatory elements (+/−100 kb or more) are listed in Table 1.

ATXN1 showed a strong enrichment of case-only variants in constrained elements (10 vs. 4; FIG. 3A and Table 17). Two dogs contributed to the case-only variants found in ATXN1 (1 JR case, seven variants; 1 SS case, three variants). Of these ten case-only variants, six (five JR case variants and one SS case variants) were found in the first intron, clustering near the 5′end (FIG. 3C). Of the remaining four, two variants were found in a SS case, one within the 3′UTR and the other in intron 3 (FIG. 3C). The other two that were identified in a JR case failed to be aligned to hg19.

CDH2, a gene previously associated with OCD in DP population [14], showed the strongest enrichment of case-only variants, not only in the DP samples (case-only vs. control-only variants, 272 vs. 118), but also in all the breeds together in the data set (242 vs. 52; FIG. 3A). This still held true when only considering case-specific variants within constrained elements (16 vs. 1; FIG. 3A). Even if DP dogs were removed from the analysis, CDH2 remained one of the top candidate genes (15 vs. 1; FIG. 3B and Table 17). Taking a closer look at the variants within constrained elements in CDH2: (i) sixteen case-only variants and only one control-only variant across all the breeds; (ii) sixteen case-only variant and six control-only variants in DP; and (iii) fifteen case-only variants and no control-only variants among all the breeds excluding DP was observed. A cluster of the case-only variants in intron2 coincided with a strong association signal from the GWA analysis (chr7:63867472, p=2.1×10-5; FIG. 3C).

CTNNA2 was partially captured in the sequence data and was enriched with twelve case-only variants within constrained elements (FIG. 3A). Two dogs, namely, a GS case (eight variants) and a JR case (five variants) contributed to the case-only variants found in CTNNA2 region. Of twelve case-only variants within CTNNA2, two variants (GS case) were found in coding region, in which one variant (in exon12) was the same as the ancestral allele and the other (in exon13) as the human allele. An intronic variant from the same dog was found between these two exons. Of the remaining nine, five variants clustered around intron 8 and exon 8. Specifically, three variants (two JR case variants and one GS case variant) clustered within intron 8, and two variants (one JR case variant and one GS case variant) around exon8. The remaining four variants reside within intron 7 and intron 9, two of which resided within DNase1 hypersensitivity site (data not shown).

PGCP was enriched with sixteen case-only variants within constrained region (FIG. 3A). The variants were identified from three dogs, namely, a SS case (six variants), a JR case (four variants), and a GS case (six variants). Of the sixteen case-only variants, one (SS case) was found within the 3′UTR and another (JR case) in exons, in which the identified variant was the same as the ancestral allele in other mammals. A deletion was also observed in exon2 (JRT case) and an intronic variant (GS case) near exon2 (FIG. 3C). The remaining twelve were distributed across PGCP, of which three failed to liftover onto hg19.

Of the 40 variants genotyped in these four genes, seven overlap chromatin marks, potentially indicating regulatory function. Four variants in CDH2 overlap H3K27Ac histone marks and/or DNase1 hypersensitivity clusters. Three of these (chr7:63845160, chr7:63852056, and chr7:63832008) are observed in OCD-risk breeds, at frequencies of 0.435, 0.050, and 0.022 respectively, and never seen in control breeds. The fourth variant (chr7:63806661) is 4-fold more common in OCD-risk breeds (frequency=0.11 vs. 0.026 in control breeds). Three variants in ATXN1 alter regions transcribed in the dog brain (K. Lindblad-Toh, unpublished RNA-Seq data), including a putative enhancer variant not seen in the control breeds (chr35:18850625, OCD-risk breed frequency=0.014). These variants, which lie in genes enriched for case-only variants, were overrepresented in cases, and alter putative regulatory elements, are strong candidates for further functional elucidation.

Single Variant Analysis

Next was to identify the top candidate functional variants in the sequencing data. Coding variants found exclusively in cases were first looked for. Most were missense mutations disrupting genes with little known relevance to brain functions (Table 22).

TABLE 22
Case-Only Missense Variants

| Chr: position | | | | | |
|---|---|---|---|---|---|
| nucleotide | | Protein | | | |
| change | dbSNP | Change | Gene | Polyphen-2 | Case samples |
| chr3: 62315616 | | p.V40I | SORCS2 | Benign | SS 5991 |
| G > A | | | | | |
| chr3: 64757837 | | p.R195H | MXD4 | Unknown | SS 5991 |
| G > A | | | | | |
| chr3: 64769048 | | p.R404S | HAUS3 | Possibly | JR 3094 |
| C > A | | | | damaging | |
| chr3: 65472187 | | p.A712V | KIAA1530 | Probably | SS 5991 |
| G > A | | | | damaging | |
| chr17: 27881676 | | p.D620N | CAPN14 | Probably | SS 5991 |
| C > T | | | | damaging | |
| chr18: 56754830 | BICF2G630690061 | p.V475I | SLC22A8 | Benign | DP 15259, JR |
| G > A | | | | | 3094 |

The variants were surveyed within protein-coding regions, including missense, nonsense, frame-shift and those located in essential splice sites. Six missense variants were detected in at least one case dog but not in any controls, two of which were predicted by Polyphen-2 [66] to change protein function with high confidence as shown in Table 19.

Both of them were present in a SS case and were located inside KIAA1530 and calpain 14 (CAPN14). KIAA153, also known as UV-stimulated scaffold protein A (UVSSA), is widely expressed in a multiplicity of dog tissues including the brain (unpublished data). While the protein is known to interact with nucleotide excision repair complex [68], its function in the brain has not been well studied, which makes it difficult to develop a functional assay for the variant. CAPN14 encodes the calcium-activated neutral proteinase 14 (calpain 14), which belongs to the calpain family that is involved in a variety of cellular processes including cell division, synaptic plasticity and apoptosis [69]. Its mRNA has been detected in several dog tissues including the brain (unpublished). However, when aligning the variant's flanking sequence to the human genome, a 187b-long sequence gap was present in the codon frame of the variant, making the translation of the impact of this variant into human difficult.

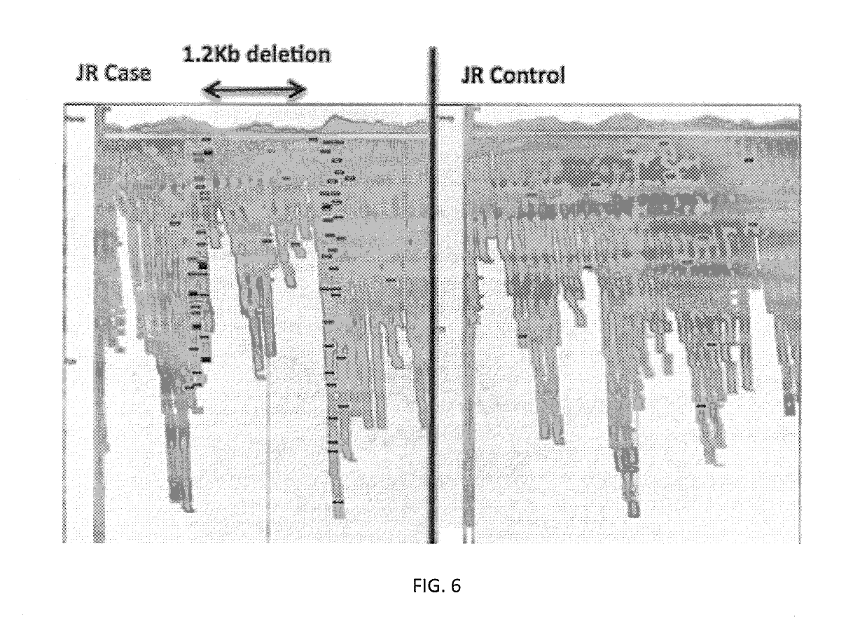

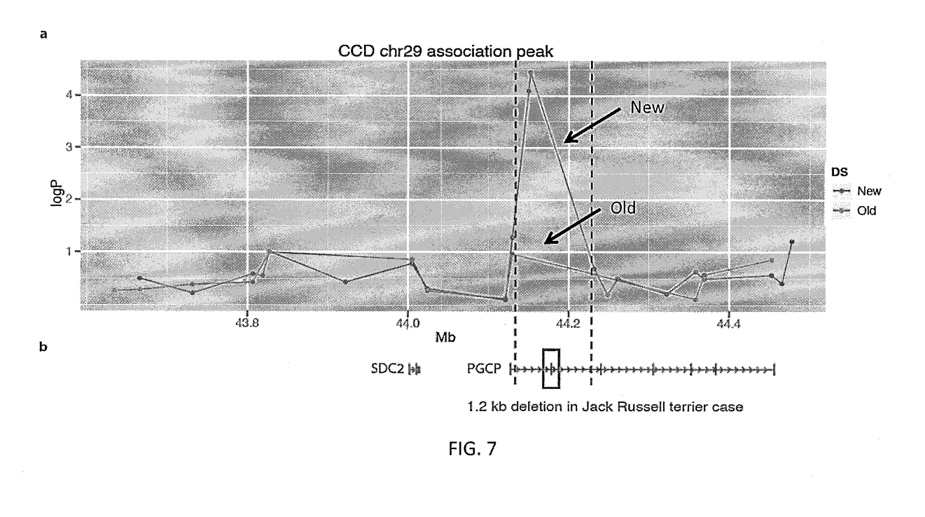

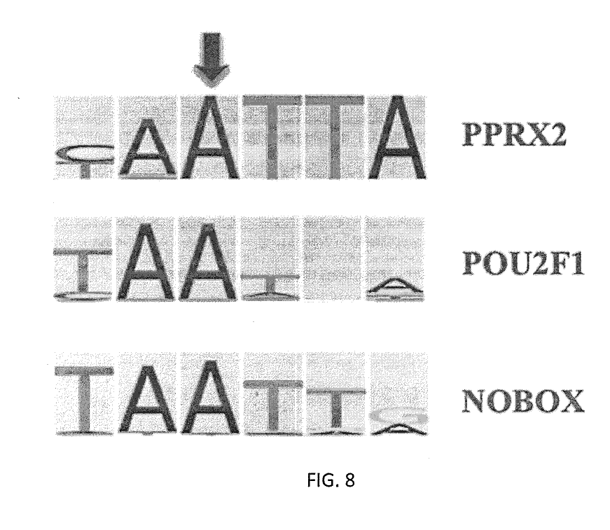

One of the two Jack Russell terrier cases had a 1.2 kb deletion (chr29:44178339-44179516; FIG. 6) overlapping exon 2 of the gene PGCP, causing a frameshift and loss of 70 amino acids from the protein. PGCP was one of the four genes enriched for case-only variants, and, while none of the DP cases had this particular deletion, a nearby SNP was among the most strongly associated in the GWAS (chr29:44152594, p=1.5×10-5, FIGS. 1 and 7). Using quantitative PCR (qPCR), the deletion in the Jack Russell terrier cases was validated and tested 74 dogs from OCD-risk breeds (including 10 unphenotyped Jack Russell terriers and 14 dogs from several breeds diagnosed with OCD) and 20 dogs from control breeds. The deletion was found in three Jack Russell terriers and in one Welsh terrier with OCD, and in none of the control breed dogs, suggesting it is associated with increased risk of OCD in multiple breeds.

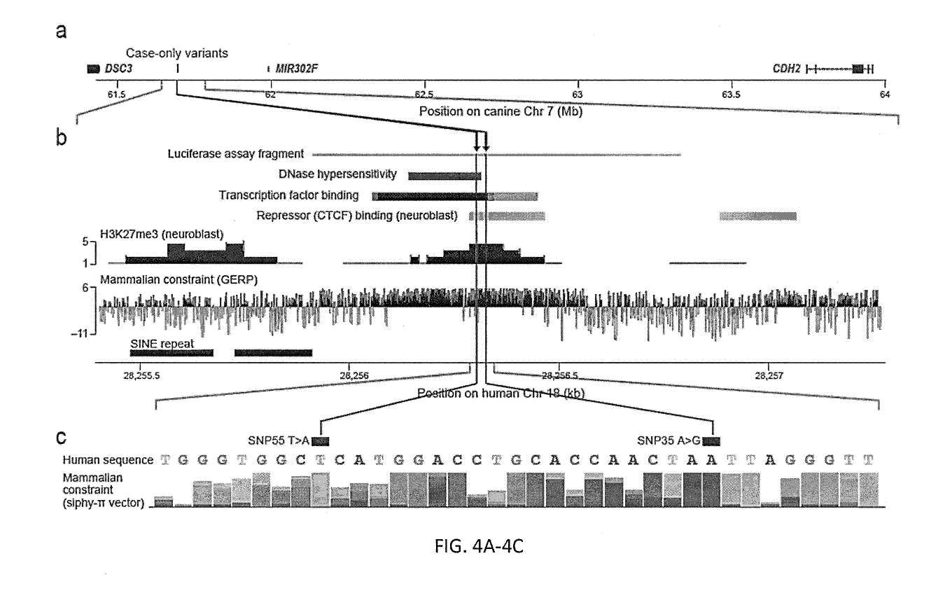

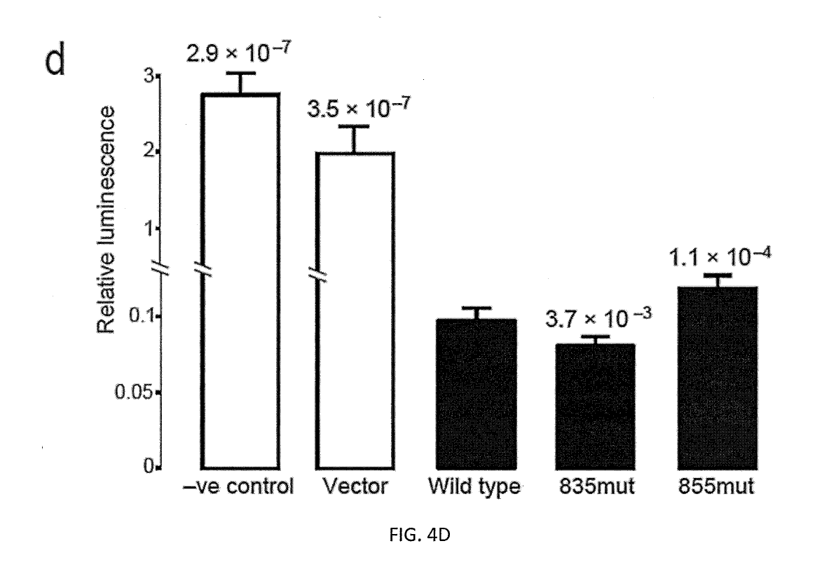

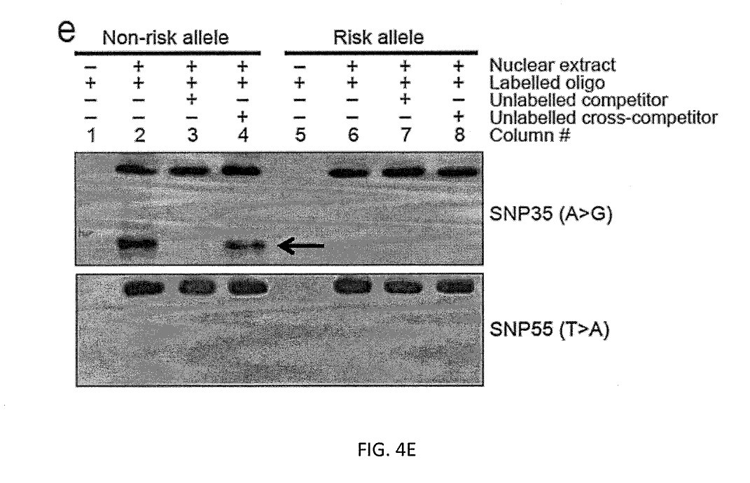

Next, non-coding variants seen only in cases were searched, focusing on 15 seen in more than one DP case. All but two are near the GWAS peak in intron 2 of CDH2 (chr7:63867472, p=2.1×10-5) reflecting the selection of DP dogs for sequencing based on their genotype at this locus. None of the 13 is obviously functional based on evolutionary constraint and histone marks. The other two variants are more interesting, changing a conserved region ˜172 kb away from an associated GWAS SNP (chr7:61865715, p=1.6×10-5), in the gene desert between the cadherin genes CDH2 and desmocolin-3 (DSC3) (FIG. 4A). The first SNP (chr7:61693835, T changed to C; SNP35 T>C) was exclusively found in 3 of 4 sequenced DP cases and showed the overall DP breed frequency of ˜0.30 in the genotyping data set. The second SNP, a private variant in the fourth Doberman case (chr7:61693855; SNP55 A>T), was just 20 bases away and alters the same highly conserved region (FIGS. 4B and 4C).

Functional Assessment of Candidate Variants

Because the region altered by SNP35 and SNP55 showed evidence of regulatory function (FIG. 4B), tests were performed to determine if the risk alleles disrupt gene expression using a luciferase reporter assay. Including the wild-type region in the reporter construct lowers expression 14-20 fold in human neuroblastoma SK-N-BE (2) cells (vector vs. wild type, t-test Bonferroni corrected p=3.5×10-7; negative control vs. wild type, t-test Bonferroni corrected p=2.9×10-7; FIG. 4D). Adding the SNP55 risk variant to the construct, however, significantly increases expression relative to the wild-type version, suggesting the regulatory element no longer functions normally (1.6-fold change, paired t-test Bonferroni corrected p=1.1×10-4; FIG. 4D). Curiously, SNP35 risk allele has the opposite effect, repressing expression even further (0.9-fold change, paired t-test, Bonferroni corrected p=3.7×10-3; FIG. 4D).

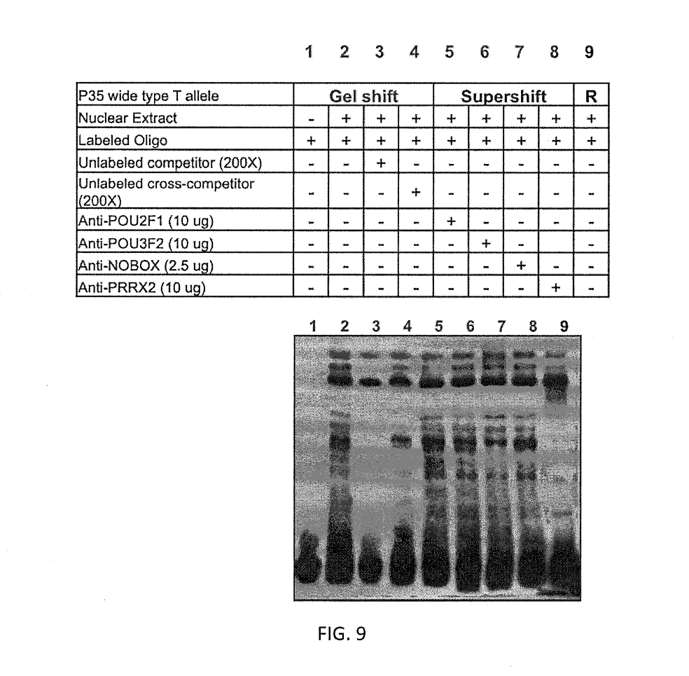

Using an electrophoretic mobility shift assay (EMSA) to examine DNA protein binding in the region, it is observed that while the SNP55 risk allele causes no apparent change relative to wild-type, the SNP35 risk allele shows markedly reduced binding (FIG. 4E). Three transcription factors are predicted by TRANSFAC [32] to bind the wild-type sequence but not the SNP35 variant (PRRX2, Oct-1 and Nobox; FIG. 8). However, there was no evidence that these three proteins bind the region in a supershift assay (FIG. 9), suggesting other factors are critical. More than 90 transcription factors are predicted to bind the wild-type sequence using various discovery tools [33]. Thus, both SNP35 and SNP55 significantly changed the silencing activity of the regulatory element, but in opposite directions and possibly through different mechanisms.

Discussion

Through a small GWAS (fewer than 90 cases and 70 controls) OCD associated loci were identified, which, particularly when analyzed together with regions of low variability, implicated specific cellular pathways in disease etiology. 9 of the top regions of association and 5 regions of fixation in 8 OCD cases and 8 breed-matched controls were sequenced. A notable excess of case-only variation in evolutionarily conserved regions was found, particularly in non-coding elements with potential regulatory function. This suggests noncoding variation is a major factor in canine OCD similar to human neuropsychiatric diseases, and unlike most artificially induced mouse models. While the dog population is comprised of >400 genetically isolated breed populations, just a small number of breeds are highly enriched for OCD, suggesting that OCD risk variants are more prevalent in these breeds. The case-only variants found in the sequence data are in fact significantly more common in OCD-risk breeds compared to breeds with no increased risk of psychiatric disorders.

By comparing the sequence data using gene-based tests, four candidate genes were identified: CDH2, CTNNA2, ATXN1, and PGCP strongly implicated for involvement in disease.

CDH2, a neural cadherin, encodes a calcium dependent cell-cell adhesion glycoprotein important for synapse assembly, where it mediates presynaptic to postsynaptic adhesions [34]. Disrupting expression of CDH2 in cultured mouse neurons causes synapse dysfunction, synapse elimination and axon retraction [35].

CTNNA2 encodes a neuronal-specific catenin protein that links cadherins to the cytoskeleton [34,36] and is associated with bipolar disorder [37], schizophrenia [38], ADHD [38] and excitement-seeking [39]. Mice with a deletion of CTNNA2 showed disrupted brain morphology and impaired startle modulation [40]. Cadherin-catenin complexes play a pivotal role in synapse formation and synaptic plasticity and therefore may be involved in the process of learning and memory [41].

ATXN1 encodes a chromatin binding protein that regulates the Notch pathway [42], a developmental pathway also active in the adult brain, where it mediates neuronal migration, morphology and synaptic plasticity [43]. Mice with a deletion of ATXN1 showed pronounced deficits in learning and memory [44].

CDH2, CTNNA2 and ATXN1 have similar spatial expression patterns in the brain and are important during brain development and for synaptic plasticity. CDH2 and CTNNA2 are highly expressed in the prefrontal cortex, amygdala, thalamus and fetal brain [34,45]. ATXN1 is highly expressed in the prefrontal cortex, basal ganglia, cerebellum and fetal brain [45,46].

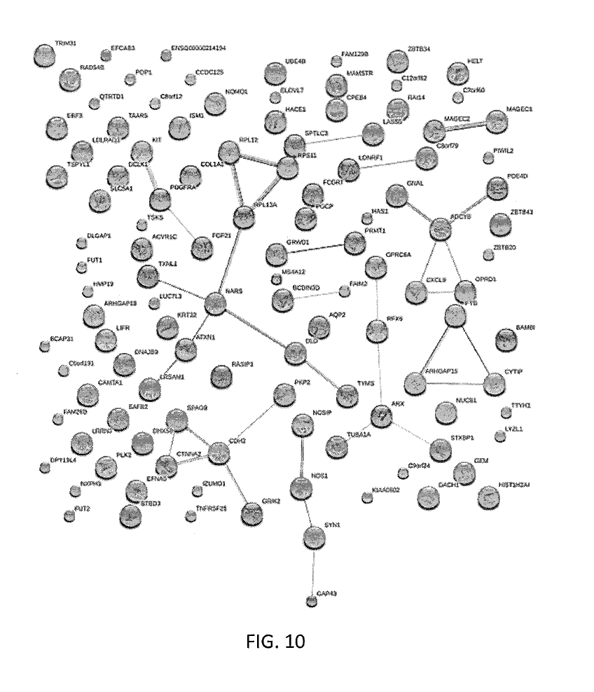

Intriguingly, the three genes appear to have functional connections to the top SNPs (association p<10-5) in a recent human OCD GWAS, which found no single associations reaching genome-wide significant, but implicated glutamatergic signaling pathways [4] (FIG. 10). Most notably, one of the top associated genes in human patients, GRIK2, encodes a glutamate receptor recruited to the synaptic membrane by CDH2/catenin complexes [47] and another top candidate, PKP2, mediates CDH2 cell adhesion and desmosomal junctions [48]. In addition, several genes whose expression levels correlate with the top human OCD-associated SNPs interact with the genes identified in dogs: LRSAM1 (cerebellum) and NARS (frontal lobe) interact with ATXN1; SPAG9 (cerebellum) acts in in developmental pathways with CDH2 and CTNNA2 [49].

The fourth gene, PGCP, encodes a poorly characterized plasma glutamate carboxypeptidase. It may help hydrolyze N-acetylaspartylglutamate (NAAG), the third most abundant neurotransmitter in the brain, to glutamate and N-acetylaspartate (NAA) [34], suggesting a potential role in glutamatergic synapse dysfunction. PGCP is associated with migraine [50], which is frequently co-morbid with OCD [51].

CDH2, CTNNA2, ATXN1, and PGCP may work in concert to regulate glutamatergic synapse formation and function in the cortico-striatal-thalamo-cortical (CSTC) brain circuit previously implicated in the pathogenesis of OCD [52-56].

Single variant analysis corroborates the hypothesis of dysregulated synapse formation in OCD. All four sequenced DP cases had one of two mutations (SNP35 and SNP55) in a regulatory region, between DSC3 and CDH2, that was shown to act as a strong silencer. The OCD-risk allele of SNP55 significantly increased the reporter gene expression while the OCD-risk allele of SNP35 had the opposite effect. While surprising, other studies have shown either deletion or reciprocal duplication of loci such as 17p11.2 and 15q13.3 can cause neuropsychiatric disorders [57]. SNP35 was confirmed using EMSA that the OCD-risk allele changes DNA binding. No change at SNP55 was observed, although in vitro assays may not capture all relevant in vivo reactions. The regulatory element is between CDH2 (2.2 Mb away) and DSC3 (0.3 Mb away), both cadherin genes involved in gamma-catenin binding (FIG. 1F), suggesting disrupted gamma-catenin binding may be an important risk factor for OCD. Additional sequence data from DSC3 (not included in the current targeted sequencing design) and more functional analysis is needed to understand the two SNPs' effects on CDH2 and DSC3.

### REFERENCES

1. Laks J, Fontenelle L F, Chalita A, Mendlowicz M V: Absence of dementia in late-onset schizophrenia: a one year follow-up of a Brazilian case series. Arquivos de neuro-psiquiatria 2006, 64:946-949.
2. van Grootheest D S, Cath D C, Beekman A T, Boomsma D I: Twin studies on obsessive-compulsive disorder: a review. Twin Res Hum Genet 2005, 8:450-458.
3. Taylor S: Molecular genetics of obsessive-compulsive disorder: a comprehensive meta-analysis of genetic association studies. Mol Psychiatry 2013, 18:799-805.
4. Stewart S E, Yu D, Scharf J M, Neale B M, Fagerness J A, Mathews C A, Arnold P D, Evans P D, Gamazon E R, Osiecki L, et al: Genome-wide association study of obsessive-compulsive disorder. Mol Psychiatry 2012, 18:788-98.
5. Welch J M, Lu J, Rodriguiz R M, Trotta N C, Peca J, Ding J D, Feliciano C, Chen M, Adams J P, Luo J, et al: Cortico-striatal synaptic defects and OCD-like behaviours in Sapap3-mutant mice. Nature 2007, 448:894-900.
6. Burguiere E, Monteiro P, Feng G, Graybiel A M: Optogenetic stimulation of lateral orbitofronto-striatal pathway suppresses compulsive behaviors. Science 2013, 340:1243-1246.
7. Zuchner S, Wendland J R, Ashley-Koch A E, Collins A L, Tran-Viet K N, Quinn K, Timpano K C, Cuccaro M L, Pericak-Vance M A, Steffens D C, et al: Multiple rare SAPAP3 missense variants in trichotillomania and OCD. Mol Psychiatry 2009, 14:6-9.
8. Overall K L: Natural animal models of human psychiatric conditions: assessment of mechanism and validity. Progress in neuro-psychopharmacology & biological psychiatry 2000, 24:727-776.
9. Overall K L, Dunham A E: Clinical features and outcome in dogs and cats with obsessive-compulsive disorder: 126 cases (1989-2000). J Am Vet Med Assoc 2002, 221:1445-1452.
10. Moon-Fanelli A A, Dodman N H, Cottam N: Blanket and flank sucking in Doberman Pinschers. J Am Vet Med Assoc 2007, 231:907-912.
11. Moon-Fanelli A A, Dodman N H, Famula T R, Cottam N: Characteristics of compulsive tail chasing and associated risk factors in Bull Terriers. J Am Vet Med Assoc 2011, 238:883-889.
12. Luescher A U: Diagnosis and management of compulsive disorders in dogs and cats. Clinical techniques in small animal practice 2004, 19:233-239.
13. Karlsson E K, Sigurdsson S, Ivansson E, Thomas R, Elvers I, Wright J, Howald C, Tonomura N, Perloski M, Swofford R: Genome-wide analyses implicate 33 loci in heritable dog neurological disorder, including regulatory variants near CDKN2A/B. Genome Biol 2013, 14:R132.
14. Dodman N H, Karlsson E K, Moon-Fanelli A, Galdzicka M, Perloski M, Shuster L, Lindblad-Toh K, Ginns E I: A canine chromosome 7 locus confers compulsive disorder susceptibility. Mol Psychiatry 2010, 15:8-10.
15. Boyko A R, Quignon P, Li L, Schoenebeck J J, Degenhardt J D, Lohmueller K E, Zhao K, Brisbin A, Parker H G, vonHoldt B M, et al: A simple genetic architecture underlies morphological variation in dogs. PLoS biology 2010, 8:e1000451.
16. Yang J, Lee S H, Goddard M E, Visscher P M: GCTA: a tool for genome-wide complex trait analysis. Am J Hum Genet 2011, 88:76-82.
17. Lee P H, O'Dushlaine C, Thomas B, Purcell S M: INRICH: interval-based enrichment analysis for genome-wide association studies. Bioinformatics 2012, 28:1797-1799.
18. Ethell I M, Hagihara K, Miura Y, Irie F, Yamaguchi Y: Synbindin, A novel syndecan-2-binding protein in neuronal dendritic spines. J Cell Biol 2000, 151:53-68.
19. Coba M P, Komiyama N H, Nithianantharajah J, Kopanitsa M V, Indersmitten T, Skene N G, Tuck E J, Fricker D G, Elsegood K A, Stanford L E, et al: TNiK is required for postsynaptic and nuclear signaling pathways and cognitive function. J Neurosci 2012, 32:13987-13999.
20. Arregui C, Pathre P, Lilien J, Balsamo J: The nonreceptor tyrosine kinase fer mediates cross-talk between N-cadherin and beta1-integrins. J Cell Biol 2000, 149:1263-1274.
21. Lee S H, Peng I F, Ng Y G, Yanagisawa M, Bamji S X, Elia L P, Balsamo J, Lilien J, Anastasiadis P Z, Ullian E M, Reichardt L F: Synapses are regulated by the cytoplasmic tyrosine kinase Fer in a pathway mediated by p120catenin, Fer, SHP-2, and beta-catenin. J Cell Biol 2008, 183:893-908.
22. Takeichi M, Abe K: Synaptic contact dynamics controlled by cadherin and catenins. Trends Cell Biol 2005, 15:216-221.
23. Vaysse A, Ratnakumar A, Derrien T, Axelsson E, Rosengren Pielberg G, Sigurdsson S, Fall T, Seppala E H, Hansen M S, Lawley C T, et al: Identification of genomic regions associated with phenotypic variation between dog breeds using selection mapping. PLoS Genet 2011, 7:e1002316.
24. Churchill L, Cotman C, Banker G, Kelly P, Shannon L: Carbohydrate composition of central nervous system synapses, Analysis of isolated synaptic junctional complexes and postsynaptic densities. Biochim Biophys Acta 1976, 448:57-72.
25. Clark R A, Gurd J W, Bissoon N, Tricaud N, Molnar E, Zamze S E, Dwek R A, McIlhinney R A, Wing D R: Identification of lectin-purified neural glycoproteins, GPs 180, 116, and 110, with NMDA and AMPA receptor subunits: conservation of glycosylation at the synapse. J Neurochem 1998, 70:2594-2605.
26. Hedges D J, Burges D, Powell E, Almonte C, Huang J, Young S, Boese B, Schmidt M, Pericak-Vance M A, Martin E, et al: Exome sequencing of a multigenerational human pedigree. PloS one 2009, 4:e8232.
27. Lindblad-Toh K, Garber M, Zuk O, Lin M F, Parker B J, Washietl S, Kheradpour P, Ernst J, Jordan G, Mauceli E, et al: A high-resolution map of human evolutionary constraint using 29 mammals. Nature 2011, 478:476-482.
28. Huang H, Chanda P, Alonso A, Bader J S, Arking D E: Gene-based tests of association. PLoS Genet 2011, 7:e1002177.
29. Siepel A, Bejerano G, Pedersen J S, Hinrichs A S, Hou M, Rosenbloom K, Clawson H, Spieth J, Hillier L W, Richards S, et al: Evolutionarily conserved elements in vertebrate, insect, worm, and yeast genomes. Genome Res 2005, 15:1034-1050.
30. Dunham I, Kundaje A, Aldred S F, Collins P J, Davis C A, Doyle F, Epstein C B, Frietze S, Harrow J, Kaul R, et al: An integrated encyclopedia of DNA elements in the human genome. Nature 2012, 489:57-74.
31. Cooper G M, Stone E A, Asimenos G, Green E D, Batzoglou S, Sidow A: Distribution and intensity of constraint in mammalian genomic sequence. Genome Res 2005, 15:901-913.
32. Wingender E, Dietze P, Karas H, Knuppel R: TRANSFAC: a database on transcription factors and their DNA binding sites. Nucleic Acids Res 1996, 24:238-241.
33. Calakos N, Patel V D, Gottron M, Wang G, Tran-Viet K N, Brewington D, Beyer J L, Steffens D C, Krishnan R R, Zuchner S: Functional evidence implicating a novel TOR1A mutation in idiopathic, late-onset focal dystonia. J Med Genet 2010, 47:646-650.
34. Pruitt K D, Tatusova T, Brown G R, Maglott D R: NCBI Reference Sequences (RefSeq): current status, new features and genome annotation policy. Nucleic Acids Res 2012, 40:D130-D135.
35. Pielarski K N, van Stegen B, Andreyeva A, Nieweg K, Jungling K, Redies C, Gottmann K: Asymmetric N-cadherin expression results in synapse dysfunction, synapse elimination, and axon retraction in cultured mouse neurons. PloS one 2013, 8:e54105.
36. Abe K, Chisaka O, Van Roy F, Takeichi M: Stability of dendritic spines and synaptic contacts is controlled by alpha N-catenin. Nat Neurosci 2004, 7:357-363.
37. Scott L J, Muglia P, Kong X Q, Guan W, Flickinger M, Upmanyu R, Tozzi F, Li J Z, Burmeister M, Absher D, et al: Genome-wide association and meta-analysis of bipolar disorder in individuals of European ancestry. Proc Natl Acad Sci USA 2009, 106:7501-7506.
38. Chu T T, Liu Y: An integrated genomic analysis of gene-function correlation on schizophrenia susceptibility genes. J Hum Genet 2010, 55:285-292.
39. Terracciano A, Esko T, Sutin A R, de Moor M H, Meirelles O, Zhu G, Tanaka T, Giegling I, Nutile T, Realo A, et al: Meta-analysis of genome-wide association studies identifies common variants in CTNNA2 associated with excitement-seeking. Translational Psychiatry 2011, 1:e49.
40. Park C, Falls W, Finger J H, Longo-Guess C M, Ackerman S L: Deletion in Catna2, encoding alpha N-catenin, causes cerebellar and hippocampal lamination defects and impaired startle modulation. Nat Genet 2002, 31:279-284.
41. Arikkath J, Reichardt L F: Cadherins and catenins at synapses: roles in synaptogenesis and synaptic plasticity. Trends Neurosci 2008, 31:487-494.
42. Tong X, Gui H, Jin F, Heck B W, Lin P, Ma J, Fondell J D, Tsai C C: Ataxin-1 and Brother of ataxin-1 are components of the Notch signalling pathway. EMBO Rep 2011, 12:428-435.
43. Ables J L, Breunig J J, Eisch A J, Rakic P: Not(ch) just development: Notch signalling in the adult brain. Nat Rev Neurosci 2011, 12:269-283.
44. Matilla A, Roberson E D, Banfi S, Morales J, Armstrong D L, Burright E N, On H T, Sweatt J D, Zoghbi H Y, Matzuk M M: Mice lacking ataxin-1 display learning deficits and decreased hippocampal paired-pulse facilitation. J Neurosci 1998, 18:5508-5516.
45. Wu C, Orozco C, Boyer J, Leglise M, Goodale J, Batalov S, Hodge C L, Haase J, Janes J, Huss J W 3rd, Su A I: BioGPS: an extensible and customizable portal for querying and organizing gene annotation resources. Genome Biol 2009, 10:R130.
46. Servadio A, Koshy B, Armstrong D, Antalffy B, Orr H T, Zoghbi H Y: Expression analysis of the ataxin-1 protein in tissues from normal and spinocerebellar ataxia type 1 individuals. Nat Genet 1995, 10:94-98.
47. Coussen F, Normand E, Marchal C, Costet P, Choquet D, Lambert M, Mege R M, Mulle C: Recruitment of the kainate receptor subunit glutamate receptor 6 by cadherin/catenin complexes. J Neurosci 2002, 22:6426-6436.
48. Barth M, Rickelt S, Noffz E, Winter-Simanowski S, Niemann H, Akhyari P, Lichtenberg A, Franke W W: The adhering junctions of valvular interstitial cells: molecular composition in fetal and adult hearts and the comings and goings of plakophilin-2 in situ, in cell culture and upon re-association with scaffolds. Cell Tissue Res 2012, 348:295-307.
49. Matthews L, Gopinath G, Gillespie M, Caudy M, Croft D, de Bono B, Garapati P, Hemish J, Hermjakob H, Jassal B, et al: Reactome knowledgebase of human biological pathways and processes. Nucleic Acids Res 2009, 37:D619-D622.
50. Anttila V, Stefansson H, Kallela M, Todt U, Terwindt G M, Calafato M S, Nyholt D R, Dimas A S, Freilinger T, Muller-Myhsok B, et al: Genome-wide association study of migraine implicates a common susceptibility variant on 8q22.1. Nature genetics 2010, 42:869-873.
51. Vasconcelos L P, Silva M C, Costa E A, da Silva Junior A A, Gomez R S, Teixeira A L: Obsessive compulsive disorder and migraine: case report, diagnosis and therapeutic approach. J Headache Pain 2008, 9:397-400.
52. Ting J T, Feng G: Glutamatergic Synaptic Dysfunction and Obsessive-Compulsive Disorder. Current Chemical Genomics 2008, 2:62-75.
53. Ahmari S E, Spellman T, Douglass N L, Kheirbek M A, Simpson H B, Deisseroth K, Gordon J A, Hen R: Repeated cortico-striatal stimulation generates persistent OCD-like behavior. Science 2013, 340:1234-1239.
54. Marsh R, Maia T V, Peterson B S: Functional disturbances within frontostriatal circuits across multiple childhood psychopathologies. Am J Psychiatry 2009, 166:664-674.
55. Milad M R, Rauch S L: Obsessive-compulsive disorder: beyond segregated cortico-striatal pathways. Trends Cogn Sci 2012, 16:43-51.
56. Pittenger C, Bloch M H, Williams K: Glutamate abnormalities in obsessive compulsive disorder: neurobiology, pathophysiology, and treatment. Pharmacol Ther 2011, 132:314-332.
57. Girirajan S, Campbell C D, Eichler E E: Human copy number variation and complex genetic disease. Annu Rev Genet 2011, 45:203-226.
58. Nestler E J, Hyman S E: Animal models of neuropsychiatric disorders. Nat Neurosci 2010, 13:1161-1169.
59. Purcell S, Neale B, Todd-Brown K, Thomas L, Ferreira M A, Bender D, Maller J, Sklar P, de Bakker P I, Daly M J, Sham P C: PLINK: a tool set for whole-genome association and population-based linkage analyses. Am J Hum Genet 2007, 81:559-575.
60. Karolchik D, Barber G P, Casper J, Clawson H, Cline M S, Diekhans M, Dreszer T R, Fujita P A, Guruvadoo L, Haeussler M, Harte R A, Heitner S, Hinrichs A S, Learned K, Lee B T, Li C H, Raney B J, Rhead B, Rosenbloom K R, Sloan C A, Speir M L, Zweig A S, Haussler D, Kuhn R M, Kent W J: The UCSC Genome Browser database: 2014 update. Nucleic Acids Res. 2013 Nov. 21. [Epub ahead of print] PMID: 24270787.
61. Picard pipeline. picard.sourceforge.net/.
62. McKenna A, Hanna M, Banks E, Sivachenko A, Cibulskis K, Kernytsky A, Garimella K, Altshuler D, Gabriel S, Daly M, DePristo M A: The Genome Analysis Toolkit: a MapReduce framework for analyzing next-generation DNA sequencing data. Genome Res 2010, 20:1297-1303.
63. DePristo M A, Banks E, Poplin R, Garimella K V, Maguire J R, Hartl C, Philippakis A A, del Angel G, Rivas M A, Hanna M, et al: A framework for variation discovery and genotyping using next-generation DNA sequencing data. Nat Genet 2011, 43:491-498.
64. Handsaker R E, Korn J M, Nemesh J, McCarroll S A: Discovery and genotyping of genome structural polymorphism by sequencing on a population scale. Nat Genet 2011, 43:269-276.
65. Robinson J T, Thorvaldsdottir H, Winckler W, Guttman M, Lander E S, Getz G, Mesirov J P: Integrative genomics viewer. Nat Biotechnol 2011, 29:24-26.
66. Adzhubei I A, Schmidt S, Peshkin L, Ramensky V E, Gerasimova A, Bork P, Kondrashov A S, Sunyaev S R: A method and server for predicting damaging missense mutations. Nat Methods 2010, 7:248-249.
67. FTP site for data files used in the study. www.broadinstitute.org/scientific-community/science/projects/mammals-models/obsessive-compulsive-disorder-ocd.
68. Sarasin A: UVSSA and USP7: new players regulating transcription-coupled nucleotide excision repair in human cells. Genome medicine 2012, 4:44.
69. Dear T N, Meier N T, Hunn M, Boehm T: Gene structure, chromosomal localization, and expression pattern of Capn12, a new member of the calpain large subunit gene family. Genomics 2000, 68:152-160.
70. Franceschini A, Szklarczyk D, Frankild S, Kuhn M, Simonovic M, Roth A, Lin J, Minguez P, Bork P, von Mering C, Jensen L J: STRING v9.1: protein-protein interaction networks, with increased coverage and integration. Nucleic acids research 2013, 41:D808-815.
71. Stewart S E, Yu D, Scharf J M, Neale B M, Fagerness J A, Mathews C A, Arnold P D, Evans P D, Gamazon E R, Osiecki L, et al: Genome-wide association study of obsessive-compulsive disorder. Molecular psychiatry 2012.

Without further elaboration, it is believed that one skilled in the art can, based on the above description, utilize the present invention to its fullest extent. The specific embodiments are, therefore, to be construed as merely illustrative, and not limitative of the remainder of the disclosure in any way whatsoever. All publications cited herein are incorporated by reference for the purposes or subject matter referenced herein.

The indefinite articles “a” and “an,” as used herein in the specification and in the claims, unless clearly indicated to the contrary, should be understood to mean “at least one.”

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method, comprising:
(a) analyzing genomic DNA from a subject for the presence of a mutation within or near
(i) a region having chromosomal boundaries/co-ordinates provided in Table 1 or 2, columns 5 and 6 of a gene selected from:
AHNAK, ATXN1, C5orf13, CAMK4, CAPN14, CHRM1, DUSP8, EPB41L4A, FAM193A, FER, FNDC3B, GALNT14, HAUS3, KIAA0232, KIAA1530, KRTAP5-8, LRRTM1, MAN2A1, MFSD10, MOB2, MXD4, NOP14, PGCP, PHACTR1, PJA2, PLD1, SLC22A6, SLC22A8, SORCS2, STX5, TADA2B, TBC1D14, TMEM212, TMEM232, TNFSF10, TNIP2, TSPYL5, WDR36, WDR74, or ZFYVE28; or
(ii) a region having chromosomal boundaries provided in Table 2A columns 4 (human) and 6 (canine) of a gene selected from:
ADD1, AHNAK, ASRGL1, ATL3, ATXN1, BLOC1S4, C4orf10, C5orf13, CAMK4, CAPN14, CCDC96, CDH2, CHRM1, CNO, CPQ, CTNNA2, DSC3, DUSP8, EPB41L4A, FAM129A, FAM193A, FER, FGFR3, FNDC3B, GALNT14, GHSR, GRPEL1, HAUS3, HCCA2, HRASLS5, INCENP, IVNS1ABP, KIAA0232, KIAA1530, KRTAP5-11, KRTAP5-2, KRTAP5-3, KRTAP5-4, KRTAP5-7, KRTAP5-8, KRTAP5-9, LETM1, LGALS12, LRRTM1, MAEA, MAN2A1, MFSD10, MOB2, MRFAP1, MXD4, NAT8L, NELFA, 0, NOP14, NREP, 0, PGCP, PHACTR1, PJA2, PLA2G16, PLD1, POLN, PPP2R2C, RNF2, RNF4, SCARNA22, SCGB1A1, SCGB1D1, SCGB1D2, SCGB2A1, SH3BP2, SLBP, SLC22A6, SLC22A8, SLC25A46, SLC3A2, SNHG1, SNORD22, SNORD30, SNORD31, SORCS2, STARD4, STX5, SWT1, TACC3, TADA2B, TBC1D14, TBC1D7, TMEM129, TMEM212, TMEM232, TNFSF10, TNIP2, TRMT1L, TSLP, TSPYL5, UVSSA, WDR36, WDR74, WHSC1, WHSC2, ZFYVE28; and
(b) identifying a subject having the mutation as a subject at elevated risk of developing or having a neuropsychiatric disorder.

2. The method of claim 1, wherein the mutation is within 100 kb, upstream or downstream, of the chromosomal boundaries/co-ordinates.

3. The method of claim 1, wherein the gene is selected from ATXN1, CHRM1, KIAA1530, NOP14, TMEM212, ZFYVE28, PGCP, or SLC22A8.

4. The method of claim 1, wherein the gene is selected from ATXN1 or PGCP.

5. The method of claim 1, wherein the mutation is within an untranslated region (UTR), intron, or exon of the gene.

6. The method of claim 1, wherein the gene is ATXN1 and the mutation is within an untranslated region (UTR), intron, or exon of ATXN1.

7. The method of claim 6, wherein the mutation is within the first intron, the 3′ UTR, or intron 3 of ATXN1.

8. The method of claim 1, wherein the gene is PGCP and the mutation is within an untranslated region (UTR), intron, or exon of PGCP.

9. The method of claim 8, wherein the mutation is within intron 2, exon 2, exon 5 or the 3′UTR of PGCP.

10. (canceled)

11. The method of claim 1, wherein the genomic DNA is analyzed using a single nucleotide polymorphism (SNP) array.

12. The method of claim 1, wherein the genomic DNA is analyzed using a bead array.

13. The method of claim 1, wherein the genomic DNA is analyzed using a nucleic acid sequencing assay.

14. The method of claim 1, wherein the subject is a human subject.

15. The method of claim 1, wherein the subject is a canine subject.

16. The method of claim 15, wherein the mutation is a SNP described in Table 3.

17.-18. (canceled)

19. The method of claim 1, wherein the neuropsychiatric disorder is obsessive-compulsive disorder.

20.-21. (canceled)

22. A method, comprising:
(a) analyzing genomic DNA from a subject for the presence of at least two mutations comprising a first mutation within a region having chromosomal boundaries/co-ordinates provided in Table 1 or 2 columns 5 and 6 of a first gene and a second mutation within a region having the chromosomal boundaries provided in Table 1 or 2, columns 5 and 6 of a second gene, wherein the first gene and second gene are selected from:
AHNAK, ATXN1, C5orf13, CAMK4, CAPN14, CDH2, CHRM1, CTNNA2, DUSP8, EPB41L4A, FAM193A, FER, FNDC3B, GALNT14, HAUS3, KIAA0232, KIAA1530, KRTAP5-8, LRRTM1, MAN2A1, MFSD10, MOB2, MXD4, NOP14, PGCP, PHACTR1, PJA2, PLD1, SLC22A6, SLC22A8, SORCS2, STX5, TADA2B, TBC1D14, TMEM212, TMEM232, TNFSF10, TNIP2, TSPYL5, WDR36, WDR74, or ZFYVE28; and
(b) identifying a subject having the at least two mutations as a subject at elevated risk of developing or having a neuropsychiatric disorder.

23.-25. (canceled)

26. The method of claim 22, wherein the first mutation is within an untranslated region (UTR), intron, or exon of the first gene and the second mutation is within an untranslated region (UTR), intron, or exon of the second gene.

27.-42. (canceled)

43. The method of claim 22, further comprising:
(c)
(i) analyzing genomic DNA from a subject for the presence of a mutation within the region between the genes CDH2 and DSC3:
(ii) analyzing genomic DNA from the subject for the presence of a mutation within intron 2 of CDH2; or
(iii) analyzing genomic DNA from the subject for the presence of a mutation within exon 8, exon 12, exon 13, intron 7, intron 8, intron 9 or intron 12 of CTNNA2; and
(d) identifying a subject having the mutation in (i), (ii), or (iii) as a subject at elevated risk of developing or having a neuropsychiatric disorder.

44.-54. (canceled)

55. A method, comprising:
(a) analyzing genomic DNA from a canine subject for the presence of a SNP in Table 3 or a mutation in a region in Table 4, 5, or 6; and
(b) identifying the canine subject having the SNP or mutation as a canine subject at elevated risk of developing or having a neuropsychiatric disorder.

56.-63. (canceled)
